(19) United States
(12) Patent Application Publication
(10) Pub. No.: US 2010/0279974 A1
(43) Pub. Date: Nov. 4, 2010

(54) Nucleosides With Non-Natural Bases as Anti-Viral Agents

(76) Inventors: Claire Pierra, Montarnaud (FR); Jean-Francois Griffon, Teyran (FR); Richard Storer, Kent (GB); Gilles Gosselin, Montpellier (FR)

Correspondence Address: JONES DAY, 222 EAST 41ST ST, NEW YORK, NY, 10017, US

(21) Appl. No.: 11/885,898

(22) PCT Filed: Mar. 9, 2006
(86) PCT No.: PCT/IB2006/002550

§ 371 Date: May 15, 2008

Related U.S. Application Data
(60) Provisional application No. 60/660,117, filed on Mar. 9, 2005.

(51) Int. Cl. A61K 31/70; C07H 19/23; C07H 19/048; C07H 19/12; A61P 31/12

(52) U.S. Cl. 514/49; 514/43; 536/27.13; 536/28.1; 536/28.3

(57) ABSTRACT

A method and composition for treating a host infected with flavivirus, pestivirus or hepacivirus comprising administering an effective flavivirus, pestivirus or hepacivirus treatment amount of a described base-modified nucleoside or a pharmaceutically acceptable salt or prodrug thereof, is provided.

### CROSS-REFERENCE TO RELATED APPLICATIONS

This application claims the benefit of priority to U.S. Provisional Application No. 60/660,117, filed on Mar. 9, 2005, the disclosure of which is incorporated herein by reference.

### FIELD OF THE INVENTION

The present invention is in the area of nucleoside derivative compounds and analogues thereof that have non-natural bases. The synthesis and use of these compounds as anti-viral and anti-cancer agents is included herein.

### BACKGROUND OF THE INVENTION

Nucleosides and nucleoside analogs are known in the art as having utility in the treatment of viral infections in mammals, including humans. Viruses that infect mammals and are treatable by the administration of pharmaceutical compositions comprising nucleosides or nucleoside derivatives include but are not limited to hepacivirus including HCV, human immunodeficiency virus (HIV), pestiviruses such as bovine viral diarrhea virus (BVDV), classic swine fever virus (CSFV, also known as hog cholera virus), and Border disease virus of sheep (BDV), and flaviviruses like dengue hemorrhagic fever virus (DHF or DENV), yellow fever virus (YFV), West Nile virus (WNV), shock syndrome and Japanese encephalitis virus (Moennig et al., Adv. Vir. Res. 1992, 41:53-98; Meyers, G. and Thiel, H-J., Adv. In Viral Res., 1996, 47:53-118; Moennig et al., Adv. Vir. Res. 1992, 41:53-98; S. B. Halstead, Rev. Infect. Dis., 1984, 6:251-64; S. B. Halstead, Science, 1988, 239:476-81; T. P. Monath, New Engl. J. Med., 1988, 319:641-3).

The family of Flaviviridae viruses include the genera pestiviruses, flaviviruses and hepacivirus. Pestivirus infections of domesticated livestock. (i.e., cattle, pigs, and sheep) cause significant economic losses worldwide. For example, BVDV causes mucosal disease in cattle and is of significant economic importance to the livestock industry (Meyers, G. and Thiel, H-J., Adv. In Viral Res., 1996, 47:53-118; Moennig et al., Adv. Vir. Res. 1992, 41:53-98).

Human pestiviruses have not been as extensively characterized as animal pestiviruses. However, serological surveys indicate considerable pestivirus exposure in humans. Pestivirus infections in man have been implicated in several diseases including congenital rain injury, infantile gastroenteritis, and chronic diarrhea in human immunodeficiency virus (HIV) positive patients (M. Giangaspero et al., Arch. Virol. Suppl., 1993, 7:53-62; M. Giangaspero et al., Int. J. Std. Aids, 1993, 4(5):300-302). The flavivirus genus includes more than 68 members that are separated into groups on the basis of serological relatedness (Calisher et al., J. Gen. Virol., 1993, 70:37-43). Clinical symptoms vary and include fever, encephalitis and hemorrhagic fever (Fields Virology, Ed.: Fields, B. N., Knipe, D. M., and Howley, P. M.; Lippincott-Raven Publishers, Philadelphia, Pa.; 1996; Chapter 31, pp. 931-59). Flaviviruses of global concern that are associated with human disease include yellow fever virus (YFV), West Nile virus (WNV), shock syndrome, Japanese encephalitis virus, and dengue hemorrhagic fever virus (DHF or DENY), (S. B. Halstead, Rev. Infect. Dis., 1984, 6:251-64; S. B. Halstead, Science, 1988, 239:476-81; T. P. Monath, New Engl. J. Med., 1988, 319:641-3).

The hepacivirus genus has hepatitis C virus (HCV) as its only species. HCV shares the same genome organization, limited sequence relatedness, and mechanism of translational control as found in the pestivirus genus (C. M. Rice, “Flaviviridae: The viruses and their replication,” Fields Virology, B. N. Fields, D. M. Knipe and P. M. Howley, Editors; 1996, Lippincott-Raven Publishers, Philadelphia, Pa.; Chpt. 30, pp. 931-59, 1005). The hepacivirus genus currently is grouped into six major genotypes and several subtypes based on an analysis of genome sequences, although this classification is becoming inadequate to describe the diversity of HCV isolates found. Also, it is unclear whether or not a relationship exists between an HCV genotype and disease severity or clinical resolution, but patients with genotype 1 have shown less response to antiviral treatments (Id.) HCV is the leading cause of chronic liver disease worldwide (N. Boyer et al., J. Hepatol. 2000, 32:98-112). It causes a slow-growing viral infection and is the major cause of cirrhosis and hepatocellular carcinoma (DiBesceglie, A. M. and B. R. Bacon, Scientific American, 1999, Oct.:80-85; N. Boyer et al., J. Hepatol. 2000, 32:98-112). About 20% of those infected clear the virus, but the remainder harbor it for life. An estimated 170 million people are infected with HCV worldwide, and about 4.5 million in the United States alone (N. Boyer et al., J. Hepatol. 2000, 32:98-112). Cirrhosis caused by chronic HCV infection occurs in 10-20% of people infected, and accounts for 8-12,000 deaths per year in the United States. HCV infection is the leading indication for liver transplant.

HCV is known to cause at least 80% of post-transfusion hepatitis and a substantial proportion of sporadic acute hepatitis. The virus is transmitted parenterally by contaminated blood and blood products, contaminated needles, and/or sexually and vertically from contaminated or infected mother to child. Preliminary evidence implicates HCV in many cases of “idiopathic” chronic hepatitis, “cryptogenic” cirrhosis, and probably hepatocellular carcinoma unrelated to other hepatitis viruses. A small proportion of healthy persons appear to be chronic HCV carriers, but this varies geographically and epidemiologically. The numbers are still preliminary, and it is unclear how many of these people have subclinical chronic liver disease (The Merck Manual, 1992, 16^{th} Ed., Chpt. 69, p. 901).

HCV is an enveloped virus containing a positive-sense, single-stranded RNA genome of approximately 9.4 k. The viral genome consists of a 5′-untranslated region (UTR), a long open reading frame (ORF) encoding a polyprotein precursor of approximately 3011 amino acids, and a short 3′-UTR. The 5′-UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region, and a serine proteinase encoded in the NS3 region. These proteinases are required for cleavage of specific regions of the precursor polyprotein into mature peptides. The carboxyl half of nonstructural protein 5, NS5, contains the RNA-dependent RNA polymerase. The function(s) of the remaining non-structural proteins, NS4A, NS4, and NS5A (the amino terminal half of non-structural protein 5) are the subjects of ongoing studies. The non-structural protein NS4A appears to be a serine protease (Hsu et al., Nat. Biotechnol., Apr. 23, 2003; [retrieved on Apr. 23, 2003]; retrieved from Entrez PubMed, Internet URL: http://www.ncbi.nlm.nih.gov/Entrez/), while studies on NS4 suggest its involvement in translational inhibition and consequent degradation of host cellular proteins (Forese et al., Virus Res., December 2002, 90(1-2):119-31). The non-structural protein NS5A has been shown to inhibit p53 activity on a p21 promoter region via its ability to bind to a specific DNA sequence, thereby blocking p53 activity (Gong et al., Zonghua Gan Zang Bing Za Zhi, Mar. 2003, 11(3):162-5). Both NS3 and NS5A have been shown to be involved with host cellular signaling transduction pathways (Giannini et al., Cell Death Diff., Jan. 2003, 10 Suppl. 1:S27-28).

Examples of antiviral agents that have been identified as active against the Flaviviridae family of viruses include:

(1) interferon and ribavirin (Battaglia, A. M. et al., Ann. Pharmacother, 2000, 34, 487-494); Berenguer, M. et al. Antivir. Ther., 1998, 3 (Suppl. 3), 125-136); this is the only current therapy recognized for treating HCV;

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p1304, 1989); Rebetol (Schering Plough) and Copegus (Roche). U.S. Pat. No. 3,798,209 and RE29,835 disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No. 4,211,771 (to ICN Pharmaceuticals) discloses the use of ribavirin as an antiviral agent.

Ribavirin reduces serum amino transferase levels to normal in 40% of patients, but it does not lower serum levels of HCV-RNA (Gary L. Davis, Gastroenterology. 118:S104-S114, 2000). Thus, ribavirin alone is not effective in reducing viral RNA levels. Additionally, ribavirin has significant toxicity and is known to induce anemia.

Interferons (IFNs) are compounds that have been commercially available for the treatment of chronic hepatitis for nearly a decade. IFNs are glycoproteins produced by immune cells in response to viral infection. IFNs inhibit viral replication of many viruses, including HCV where it may work through the viral NS5A region that is known to interact with the protein kinase, PKR, an IFN-mediator (M. Major et al., “Hepatitis C Viruses,” Fields Virology, B. N. Fields, D. M. Knipe and P. M. Howley, Editors; 2001, Lippincott-Raven Publishers, Philadelphia, Pa.; Chpt. 34, pp. 1127-61). When used as the sole treatment for hepatitis C infection, IFN suppresses serum HCV-RNA to undetectable levels. Additionally, IFN normalizes serum amino transferase levels. Unfortunately, the effects of IFN are temporary and a sustained response occurs in only 8%-9% of patients chronically infected with HCV (Gary L. Davis. Gastroenterology 118:S104-S114, 2000). In addition, IFN therapies are associated with severe and unpleasant side-effects such as nausea and vomiting.

A number of patents disclose HCV treatments using interferon-based therapies. For example, U.S. Pat. No. 5,980,884 to Blatt et al. discloses methods for retreatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,908,621 to Glue et al. discloses the use of polyethylene glycol modified interferon for the treatment of HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696.

Schering-Plough sells ribavirin as Rebetol® capsules (200 mg) for administration to patients with HCV. The U.S. FDA has approved Rebetol capsules to treat chronic HCV infection in combination with Schering's alpha interferon-2b products Intron® A and PEG-Intron™. Rebetol capsules are not approved for monotherapy (i.e., administration independent of Intron® A or PEG-Intron), although Intron A and PEG-Intron are approved for monotherapy (i.e., administration without ribavirin). Hoffman La Roche is selling ribavirin under the name CoPegus in Europe and the United States, also for use in combination with interferon for the treatment of HCV. Other alpha interferon products include Roferon-A (Hoffmann-La Roche), Infergen® (InterMune, formerly Amgen's product), and Wellferon® (Wellcome Foundation) are currently FDA-approved for HCV monotherapy. Interferon products currently in development for HCV include: Roferon-A (interferon alfa-2a) by Roche, PEGASYS (pegylated interferon alfa-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

The combination of IFN and ribavirin for the treatment of HCV infection has been reported to be effective in the treatment of IFN naïve patients (Battaglia, A. M. et al., Ann. Pharmacother. 34:487-494, 2000). Combination treatment is effective both before hepatitis develops and when histological disease is present (Berenguer, M. et al. Antivir. Ther. 3(Suppl. 3):125-136, 1998). Currently, the most effective therapy for HCV is combination therapy of pegylated interferon with ribavirin (2002 NIH Consensus Development Conference on the Management of Hepatitis C). However, the side effects of combination therapy can be significant and include hemolysis, flu-like symptoms, anemia, and fatigue (Gary L. Davis. Gastroenterology 118:S104-S114, 2000).

(2) Substrate-based NS3 protease inhibitors (Attwood et al., Antiviral peptide derivatives, PCT WO 98/22496, 1998; Attwocid et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734).

(3) Non-substrate-based inhibitors such as 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing apara-phenoxyphenyl group;

(4) Thiazolidine derivatives which show relevant inhibition in a reverse-phase HPLC assay with an NS3/4A fusion protein and NS5AJ5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193;

(5) Thiazolidines and benzanilides identified in Kakiuchi N. et al. J. EBS Letters 421, 217-220; Takeshita N. et al. Analytical Biochemistry, 1997, 247, 242-246;

(6) A phenanthrenequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griseofulvum, which demonstrates activity in a scintillation proximity assay (Chu M. et al., Bioorganic and Medicinal Chemistry Letters 9, 1949-1952);

(7) Selective NS3 inhibitors based on the macromolecule elgin c, isolated from leech (Qasim M. A. et al., Biochemistry, 1997, 36, 1598-1607);

(8) Helicase inhibitors (Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554);

(9) Polymerase inhibitors such as:
(i) nucleotide analogues, for example, gliotoxin (Ferrari R. et al. Journal of Virology, 1999, 73, 1649-1654);
(ii) the natural product cerulenin (Lohmann V. et al., Virology, 1998, 249, 108-118); and
(iii) non-nucleoside polymerase inhibitors, including compound R803 (WO 04/018463 A2 and WO 03/040112 A1, both to Rigel Pharmaceuticals, Inc.); substituted diamine pyrimidines (WO 03/063794 A2 to Rigel Pharmaceuticals, Inc.); benzimidazole derivatives (Bioorg. Med. Chem. Lett., 2004, 14:119-124 and Bioorg. Med. Chem. Lett., 2004, 14:967-971, both to Boehringer Ingelheim Corporation; N,N-disubstituted phenylalanines (J. Biol. Chem., 2003, 278:9495-98 and J. Med. Chem., 2003, 13:1283-85, both to Shire Biochem, Inc.; substituted thiophene-2-carboxylic acids (Bioorg. Med. Chem. Lett., 2004, 14:793-796 and Bioorg. Med. Chem. Lett., 2004, 14:797-800, both to Shire Biochem, Inc.); α,γ-diketoacids (J. Med. Chem., 2004, 14-17 and WO 00/006529 A1, both to Merck & Co., Inc.; and meconic acid derivatives (Bioorg. Med. Chem. Lett., 2004, 3257-3261, WO 02/006246 A1 and WO03/062211 A1, all to IRBM Merck & Co., Inc.);

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257).

(11) Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. R-10101591).

(12) Nuclease-resistant ribozymes (Maccjak, D. J. et al., Hepatology 1999, 30, abstract 995).

(13) Nucleoside analogs have also been developed for the treatment of Flaviviridae infections.

Idenix Pharmaceuticals discloses branched nucleosides, and their use in the treatment of HCV and flaviviruses and pestiviruses in U.S. Pat. No. 6,812,219 and in International Publication Nos. WO 01/90121 (filed May 23, 2001) and

WO 01/92282 (filed May 26, 2001). A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier.

Other patent applications disclosing the use of certain nucleoside analogs to treat hepatitis C virus include: PCT/CA00/01316 (WO 01/32153; Nov. 3, 2000) and PCT/CA01/00197 (WO 01/60315; Feb. 19, 2001) filed by BioChem Pharma, Inc. (now Shire Biochem, Inc.); PCT/US02/01531 (WO 02/057425; Jan. 18, 2002) and PCT/US02/03086 (WO 02/057287; Jan. 18, 2002) filed by Merck & Co., Inc.; PCT/EP01/09633 (WO 02/18404; published Aug. 21, 2001) and WO 02/100415 A2 filed by Roche; PCT Publication No. WO 01/79246 (Apr. 13, 2001) and WO 02/32920 (Oct. 18, 2001) by Pharmasset, Inc.; WO 03/062256 A1, WO 03/0622255 A2, and WO 03/062257 A1, all by Ribapharm, Inc.; and WO 03/093290 A2 by Genelabs Technologies, Inc.

Toyama Chemical Co., Ltd., discloses antiviral nucleosides that have a pyrazine-carboxamido, pyrazine-amidino, or pyrazine-thioamino base (U.S. Pat. No. 6,800,629). Toyama further discloses that the 5′-triphosphate form of its T-1106 nucleoside exhibits antiviral activity in vivo, but the non-phosphorylated nucleoside form appears to be inactive (44^{th} ICACC Meeting, Washington, D.C., Oct. 30-Nov. 2, 2004; Abst. No. F-487).

(14) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.).

(15) Other compounds currently in clinical development for treatment of hepatitis C virus include: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

Anti-viral purines that have acyclic substituents are known and have been used to treat various viral infections. Perhaps best known of this class of compounds are acyclovir, ganciclovir, famciclovir, penciclovir, adefovir and adefovir dipivoxil, all of which are useful in the treatment of human syncytial virus (HSV), cytomegalo virus (CMV), and varicella-zoster virus (see EP 0 72027 to the Wellcome Foundation Ltd., UK, for treatment of equine rhinopneumonitis virus; JP 06227982 to Ajinomoto KK, for treatment of varicella-zoster virus and cytomegalovirus; S. Vittori et al., Deaza-and Deoxyadenosine Derivatives: Synthesis and Inhibition of Animal Viruses as Human Infection Models, in Nucleosides, Nucleotides & Nucleic Acids (2003) 22(5-8): 877-881, for treatment of bovine herpes virus 1 (BHV-1) and sheep Maedi-Visna Virus (MVV); R. Wang et al., Synthesis and biological activity of 2-aminopurine methylenecyclopropane analogues of nucleosides, in Nucleosides, Nucleotides & Nucleic Acids (2003) 22(2): 135-144, for treatment of HSV-1 and HBV; U.S. Pat. No. 6,444,656 to BioChem Pharma, Inc., Canada, for treatment of HIV and/or HBV infections; and WO 02/057288 to LG Chem Investment Ltd. for acyclic nucleoside phosphonate compounds for use as anti-HBV agents).

Drug-resistant variants of viruses can emerge after prolonged treatment with an antiviral agent. Drug resistance most typically occurs by mutation of a gene that encodes for an enzyme used in viral replication, and, for example, in the case of HIV, reverse transcriptase, protease, or DNA polymerase. It has been demonstrated that the efficacy of a drug against viral infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameter of the drug can be altered by such combination or alternation therapy. In general, combination therapy is typically preferred over alternation therapy because it induces multiple simultaneous pressures on the virus. One cannot predict, however, what mutations will be induced in the viral genome by a given drug, whether the mutation is permanent or transient, or how an infected cell with a mutated viral sequence will respond to therapy with other agents in combination or alternation. This is exacerbated by the fact that there is a paucity of data on the kinetics of drug resistance in long-term cell cultures treated with modern antiviral agents.

A significant focus of current antiviral research is directed to the development of improved methods of treatment of chronic HCV infections in humans (DiBesceglie, A. M. and Bacon, B. R., Scientific American, Oct.: 80-85, (1999)).

In view of the severity of diseases associated with pestiviruses, flaviviruses, and hepatitis C virus, and their pervasiveness in animals and humans, it is an object of the present invention to provide a compound, method and composition for the treatment of a host infected with any member of the family Flaviviridae, including hepatitis C virus.

Thus, it is another object of the present invention to provide a method and pharmaceutically-acceptable composition for the prophylaxis and treatment of a host, and particularly a human, infected with any member of the family Flaviviridae.

It is still another object of the invention to provide nucleoside compounds that have optionally substituted non-natural base members and congeners thereof, or a physiologically acceptable salt, ester or prodrug thereof, for the manufacture of a medicament to be used in the prophylaxis or treatment of a host infected with a pestivirus, flavivirus or hepatitis C virus.

### SUMMARY OF THE INVENTION

Compounds, methods and compositions for the treatment of a host infected with a flavivirus, pestivirus or hepacivirus infection are described that includes an effective treatment amount of a β-D- or β-L-nucleoside of the Formulae (i)-(ii) and (iv)-(xxiii), or a pharmaceutically acceptable salt or prodrug thereof. In one embodiment, the virus is hepatitis C.

Methods and compositions for the treatment of pestivirus, flavivirus and hepacivirus infections are described that include administering an effective amount of a nucleoside compound of the general Formulae (i), (ii), (iv), (v), (vi), (vii), (viii), (ix), (x), (xi), (xii), (xiii), (xiv), (xv), (xvi), (xvii), (xviii), (xix), (xx), (xxi), (xxii), (xxiii), or (xxiv):

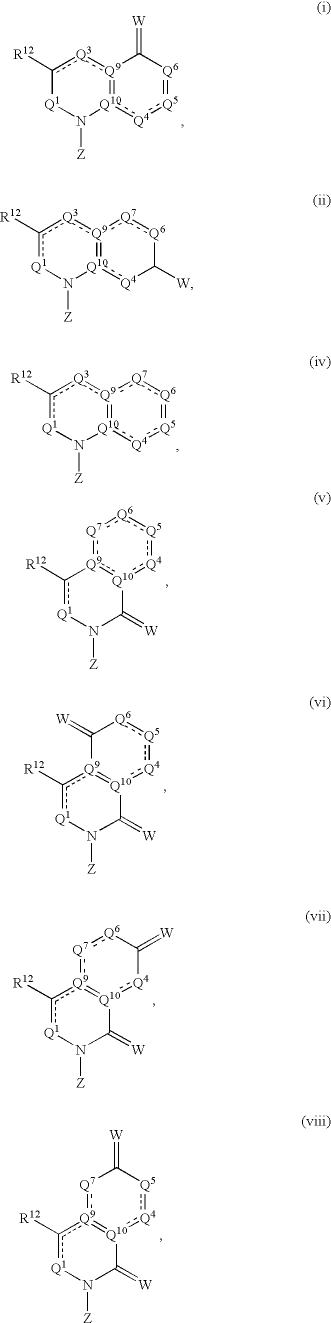

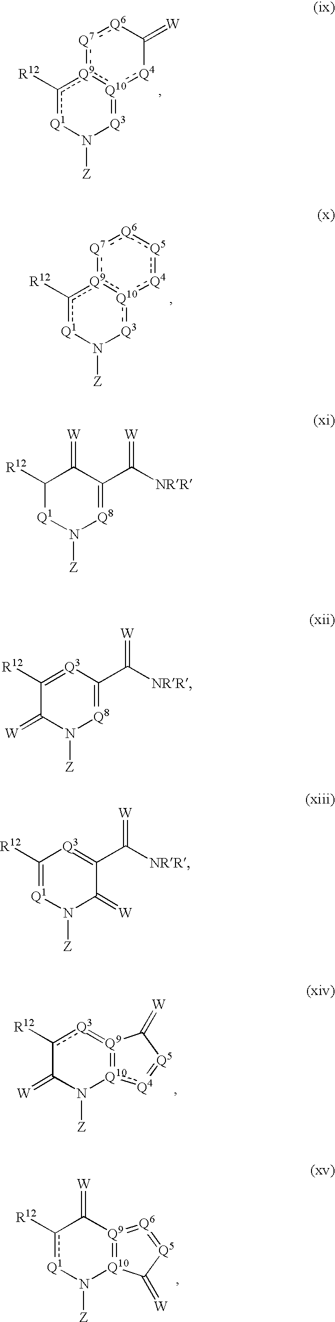

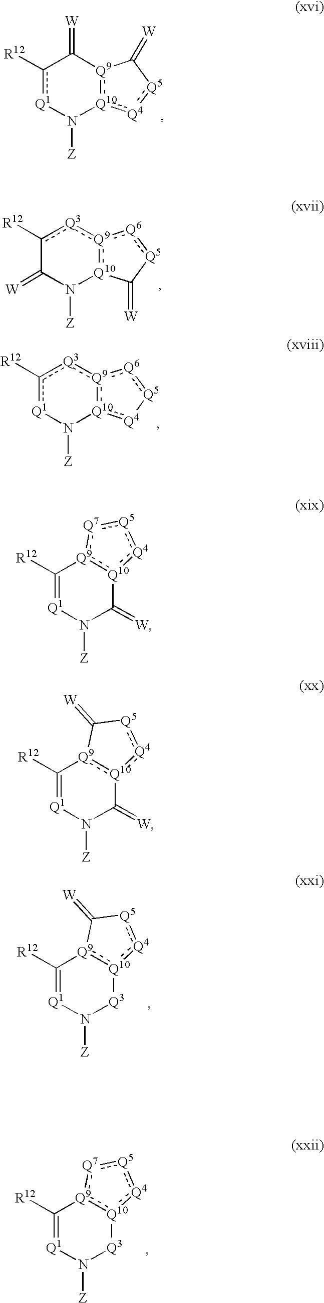

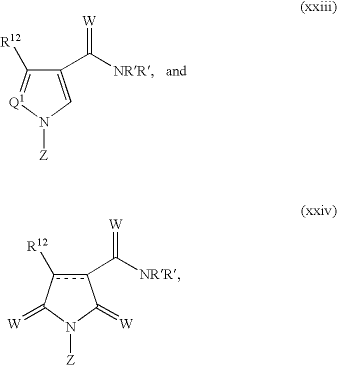

wherein:
Each W is independently O, S or N—R;
Q¹, Q³, Q⁴, Q⁵, Q⁶, Q⁷, Q⁸, Q⁹, and Q¹⁰, each independently, is C—R, N—R or N to provide appropriate valence; and
Each R is independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═)NH₂, C(═O)NHR⁴, C(═O)N(R⁴)₂, or N₃;
R′ is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
[symbol] indicates the presence of a single or double bond;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═)NH₂, C(═O)NHR⁴, C(═O)N(R⁴)₂, or N₃;
Each Y³ is independently H, F, Cl, Br or I; and
Z is selected from the group consisting of Formulae (I), (II), (III), and (IV):

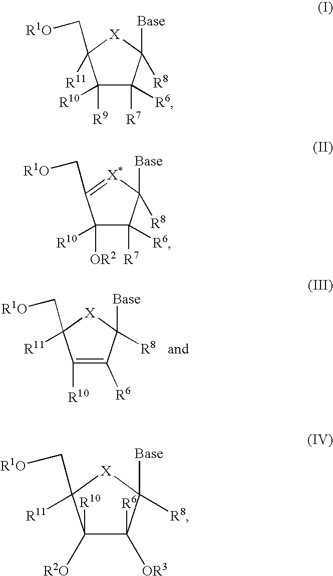

wherein:

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂: C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, alkynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂; —NH(lower alkyl), —NH(acyl), —N(lower alkyl)₂, —N(acyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2; and

all tautomers, stereoisomers and enantiomeric forms thereof; or

a pharmaceutically acceptable salt or prodrug thereof,

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system;

further provided that in Formulae (i)-(ii), Q⁴ and Q⁶ are not simultaneously both N and Q³ and Q⁷ are not C—OH; and

that in Formula (xviii) Q⁵ and Q⁶ are not simultaneously both N or N—R.

In a first principal embodiment, a method for the treatment of a host infected with a flavivirus, pestivirus or hepacivirus, and in particular HCV, infection is provided, comprising administering an effective treatment amount of a compound of base Formulae (i), (ii), or (iv) wherein Z is selected from the group consisting of Formulae (I), (II), (III), (IV) and (V):

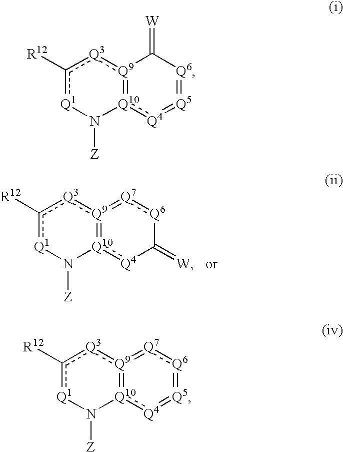

or a pharmaceutically acceptable salt or prodrug thereof, wherein:
W is O, S or N—R;
Q¹, Q³, Q⁴, Q⁵, Q⁶, Q⁷, Q⁸, Q⁹, and Q¹⁰, each independently, is C—R or N; and
Each R is independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
[symbol] indicates the presence of a single or double bond;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═)NH₂, C(═O)NHR⁴, C(═O)N(R⁴)₂, or N₃;
Each Y³ is independently H, F, Cl, Br or I;
Z is selected from the group consisting of Formulae (I), (II), (III), and (IV),

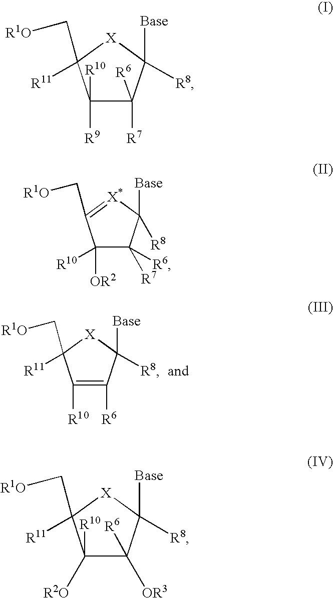

wherein,

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, alkynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂, —NH(lower alkyl), —NH(acyl), —N(lower alkyl)₂, —N(acyl)₂ᵢ (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric forms thereof; or

a pharmaceutically acceptable salt or prodrug thereof,

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system;

further provided that in Formulae (i)-(ii), Q⁴ and Q⁶ are not simultaneously both N and Q³ and Q⁷ are not C—OH.

In a second principal embodiment, a method for the treatment of a host infected with a flavivirus, pestivirus or hepacivirus, and in particular HCV, infection is provided, comprising administering an effective treatment amount of a compound of base Formulae (v)-(x) wherein Z is selected from the group consisting of Formulae (I), (II), and (IV):

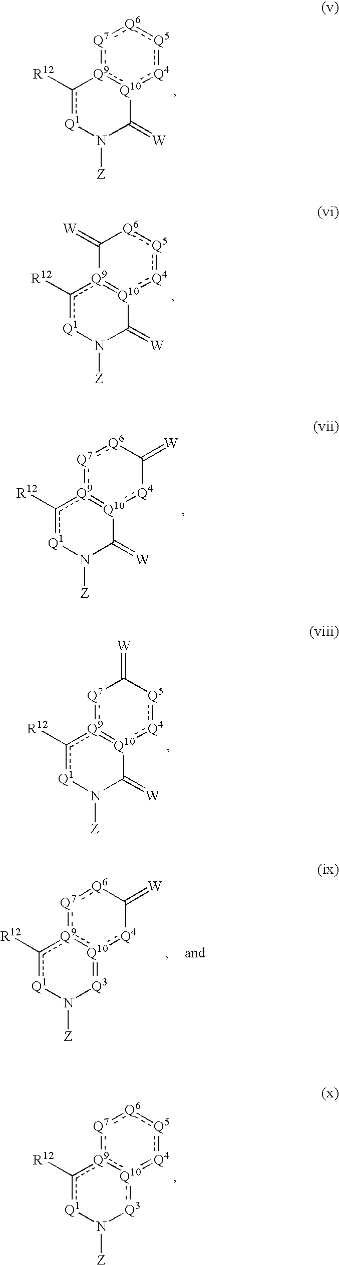

wherein
W, Q¹, Q³, Q⁴, Q⁶, Q⁶, Q⁷, Q⁸, Q⁹, Q¹⁰, R, R⁴, R⁵, Y³, R¹, R², R³, R⁶, R¹⁰, R⁷, R⁹, R⁸, R¹¹, R¹², X, X*, m and Z all are as defined above;
[symbol] indicates the presence of a single or a double bond;
all tautomers, stereoisomers and enantiomeric forms thereof; or
a pharmaceutically acceptable salt or prodrug thereof,
provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a third principal embodiment, a method for the treatment of a host infected with a flavivirus, pestivirus or hepacivirus, and in particular HCV, infection is provided, comprising administering an effective treatment amount of a compound of base Formulae (xi)-(xiii) wherein Z is selected from the group consisting of Formulae (I), (II), (III), and (IV):

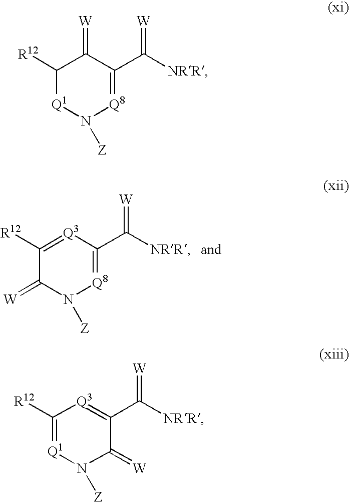

wherein,
Q¹, Q³, Q⁸, R, R′, R⁴, R⁵, Y³, R¹, R², R³, R⁶, R¹⁰, R⁷, R⁹, R⁸, R¹¹, R¹², X, X*, m and Z all are as defined above; and
all tautomers, stereoisomers and enantiomeric forms thereof; or
a pharmaceutically acceptable salt or prodrug thereof,

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a fourth principal embodiment, a method for the treatment of a host infected with a flavivirus, pestivirus or hepacivirus, and in particular HCV, infection is provided, comprising administering an effective treatment amount of a compound of base Formulae (xiv)-(xviii) wherein Z is selected from the group consisting of Formulae (I), (II), (III), and (IV):

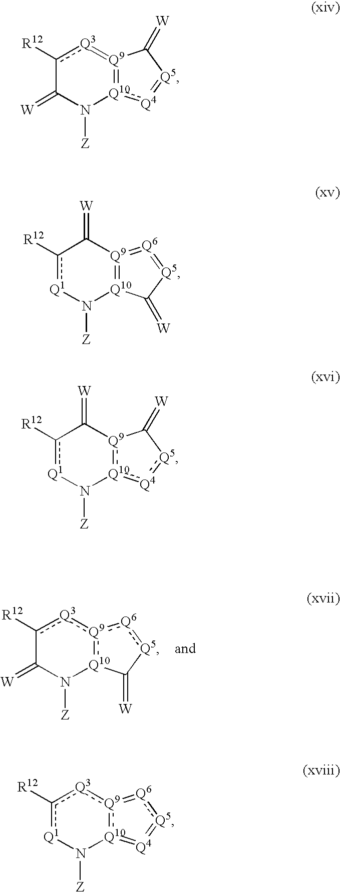

wherein,
W, Q¹, Q³, Q⁴, Q⁵, Q⁶, Q⁷, Q⁸, Q⁹, Q¹⁰, R, R⁴R⁵, Y³, R¹, R², R³, R⁶, R¹⁰, R⁷, R⁹,
   R⁸, R¹¹, R¹², X, X*, m and Z all are as defined above;
[symbol] indicates the presence of a single or a double bond;
all tautomers, stereoisomers and enantiomeric forms thereof; or
a pharmaceutically acceptable salt or prodrug thereof,
provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system; and
further provided that in Formula (xviii) Q⁵ and Q⁶ are not simultaneously both N or N—R.

In a fifth principal embodiment, a method for the treatment of a host infected with a flavivirus, pestivirus or hepacivirus, and in particular HCV, infection is provided, comprising administering an effective treatment amount of a compound of base Formulae (xix)-(xxii) wherein Z is selected from the group consisting of Formulae (I), (II), (III), and (IV):

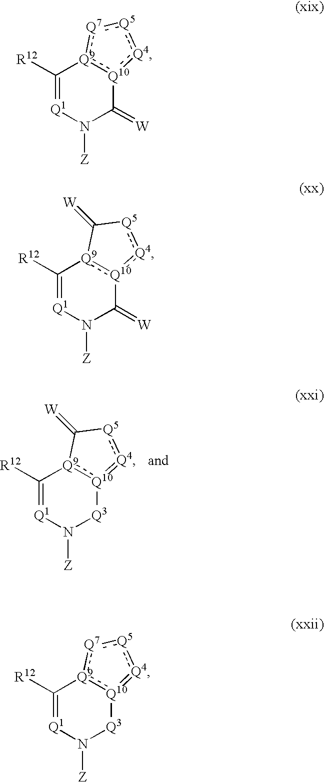

wherein:
W, Q¹, Q³, Q⁴, Q⁵, Q⁷, Q⁹, Q¹⁰, R, R⁴, R⁵, Y³, R¹, R², R³, R⁶, R¹⁰, R⁷, R⁹,
   R⁸, R¹¹, R¹², X, X*, m and Z all are as defined above;
[symbol] indicates the presence of a single or a double bond;
all tautomers, stereoisomers and enantiomeric forms thereof; or
a pharmaceutically acceptable salt or prodrug thereof,
   provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a sixth principal embodiment, a method for the treatment of a host infected with a flavivirus, pestivirus or hepacivirus, and in particular HCV, infection is provided, comprising administering an effective treatment amount of a compound of base Formulae (xxiii)-(xxiv) wherein Z is selected from the group consisting of Formulae (I), (II), (III), and (IV):

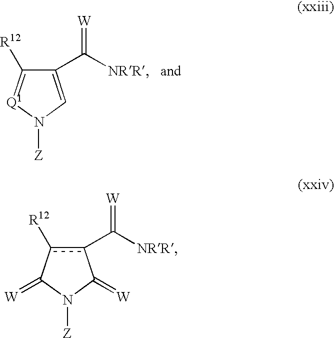

wherein,
W, Q¹, R, R′, R⁴, R⁵, Y³, R¹, R², R³, R⁶, R¹⁰, R⁷, R⁹, R⁸, R¹¹, R¹², X, X*, m and Z all are as defined above; and
all tautomers, stereoisomers and enantiomeric forms thereof; or
a pharmaceutically acceptable salt or prodrug thereof,
provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

The β-D- and β-L-nucleosides of this invention inhibit flavivirus, pestivirus or hepacivirus activity, and can be assessed for their ability to do so by standard screening methods.

In one embodiment the efficacy of the anti-flavivirus, pestivirus or hepacivirus compound is measured according to the concentration of compound necessary to reduce the plaque number of the virus in vitro, according to methods set forth more particularly herein, by 50% (i.e. the compound's EC₅₀). In preferred embodiments the compound exhibits an EC₅₀ of less than 15 or preferably, less than 10 micromolar in vitro.

In another embodiment, the active compound can be administered in combination or alternation with one or more other anti-flavivirus, pestivirus or hepacivirus agent. A variety of known antiviral agents can be used in this context. In combination therapy, effective dosages of two or more agents are administered together, whereas during alternation therapy an effective dosage of each agent is administered serially. The dosages will depend on absorption, inactivation and excretion rates of the drug as well as other factors known to those of skill in the art. It is to be noted that dosage values will also vary with the severity of the condition to be alleviated. Further, it is to be understood that for any particular subject, specific dosage regimens and schedules should be adjusted over time according to the individual need and the professional judgment of the person administering or supervising the administration of the compositions.

HCV is a member of the Flaviviridae family; however, now, HCV has been placed in a new monotypic genus, hepacivirus. Therefore, in one embodiment, the flavivirus or pestivirus is not HCV. However, in a separate embodiment, the virus is a hepacivirus, and in a preferred embodiment, is HCV.

### BRIEF DESCRIPTION OF THE DRAWINGS

FIG. 1 depicts illustrative examples of compound species of the present invention.

### DETAILED DESCRIPTION OF THE INVENTION

The invention as disclosed herein is a compound, method and composition for the treatment of flavivirus, pestivirus or hepacivirus, and in particular HCV, infection in humans and other host animals, that includes the administration of an effective flavivirus, pestivirus or hepacivirus treatment amount of an β-D- or β-L-nucleoside as described herein or a pharmaceutically acceptable salt or prodrug thereof, optionally in a pharmaceutically acceptable carrier, and further optionally in combination or alternation with at least one other anti-viral agent as provided in the Background of this specification. The compounds of this invention either possess antiviral (i.e., flavivirus, pestivirus or hepacivirus, and in particular HCV) activity, or are metabolized to a compound that exhibits such activity.

The following features are found in the present invention:
(a) β-D- or β-L-nucleosides of the Formulae (i)-(ii) and (iv)-(xxiii), and a pharmaceutically acceptable salt, ester and/or prodrug thereof;
(b) β-D- and β-L-nucleosides of Formulae (i)-(ii) and (iv)-(xxiii), and a pharmaceutically acceptable salt, ester and/or prodrug thereof, for use in the treatment or prophylaxis of a flavivirus, pestivirus or hepacivirus infection, especially in individuals diagnosed as having a flavivirus, pestivirus or hepacivirus infection or being at risk for becoming infected by flavivirus, pestivirus or hepacivirus;
(c) use of the β-D- and β-L-nucleosides of Formulae (i)-(ii) and (iv)-(xxiii), and a pharmaceutically acceptable salt, ester, and/or prodrug thereof, in the manufacture of a medicament for treatment of a flavivirus, pestivirus or hepacivirus infection;
(d) a pharmaceutical formulation comprising the β-D- and β-L-nucleosides of Formulae (i)-(ii) and (iv)-(xxiii), and a
   pharmaceutically acceptable salt, ester, and/or prodrug thereof, optionally together with a pharmaceutically acceptable carrier or diluent, and further optionally provided in combination or alternation with at least one other anti-viral agent as provided in this specification;
(e) a β-D- and β-L-nucleoside of Formulae (i)-(ii) and (iv)-(xxiii), and a pharmaceutically acceptable salt, ester, and/or prodrug thereof, substantially in the absence of enantiomers of the described nucleoside, or substantially isolated from other chemical entities;
(f) a process for the preparation of a β-D- and β-L-nucleoside of Formulae (i)-(ii) and (iv)-(xxiii), and a pharmaceutically acceptable salt, ester, and/or prodrug thereof; and

(g) a process for the preparation of a β-D- and β-L-nucleoside of Formulae (i)-(ii) and (iv)-(xxiii), and a pharmaceutically acceptable salt, ester, and/or prodrug thereof, substantially in the absence of enantiomers of the described nucleoside, or substantially isolated from other chemical entities.

Flaviviruses included within the scope of this invention are discussed generally in Fields Virology, Editors: Fields, B. N., Knipe, D. M., and Howley, P. M., Lippincott-Raven Publishers, Philadelphia, Pa., Chapter 31, 1996. Specific flaviviruses include, without limitation: Absettarov, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, Royal Farm, Russian spring-summer encephalitis, Saboya, St. Louis encephalitis, Sal Vieja, San Perlita, Saumarez Reef, Sepik, Sokuluk, Spondweni, Strafford, Tembusu, Tyuleniy, Uganda S, Usutu, Wesselsbron, West Nile, Yaounde, Yellow fever, and Zika.

Pestiviruses included within the scope of this invention are discussed generally in Fields Virology, Editors: Fields, B. N., Knipe, D. M., and Howley, P. M., Lippincott-Raven Publishers, Philadelphia, Pa., Chapter 33, 1996. Specific pestiviruses include, without limitation: bovine viral diarrhea virus (“BVDV”), classical swine fever virus (“CSFV,” also called hog cholera virus), and border disease virus (“BDV”).

The hepacivirus group (hepatitis C virus; HCV) consists of a number of closely related but genotypically distinguishable viruses that infect humans. There are approximately 6 HCV genotypes and more than 50 subtypes. Due to the similarities between pestiviruses and hepaciviruses, combined with the poor ability of hepaciviruses to grow efficiently in cell culture, bovine viral diarrhea virus (BVDV) is often used as a surrogate to study the HCV virus.

### I. ACTIVE COMPOUNDS OF THE PRESENT INVENTION

In a first principal embodiment, a method for the treatment of a host infected with a flavivirus, pestivirus or hepacivirus, and in particular HCV, infection is provided, comprising administering an effective treatment amount of a compound of Formulae (i)-(ii), and (iv):

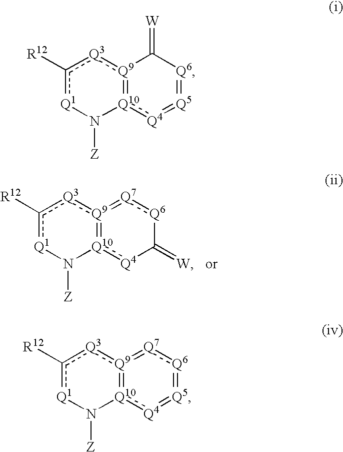

wherein:
W is O, S or N—R;
Q¹, Q³, Q⁴, Q⁵, Q⁶, Q⁷, Q⁹, and Q¹⁰, each independently, is C—R, N—H, or N; and
R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═)NH₂, C(═O)NHR⁴, C(═O)N(R⁴)₂, or N₃;
[symbol] indicates the presence of a single or double bond;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, or hydroxyalkyl;
R¹² is H, halo, alkyl, alkenyl, alkynyl, alkoxy, alkoxyalkyl, hydroxyalkyl, cycloalkyl, nitro, cyano, OH, OR⁴, NH₂, Mile, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
Each Y³ is independently H, F, Cl, Br or I; and
Z is selected from the group consisting of Formulae (I), (II), (III), and (IV):

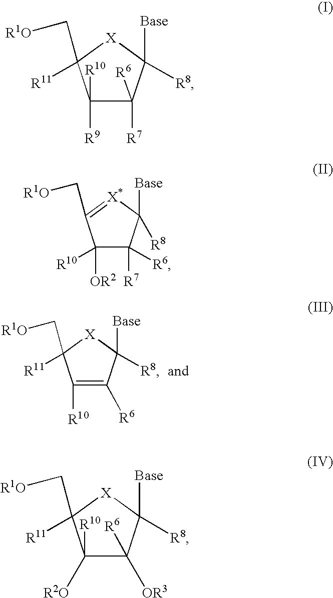

wherein:

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-allynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, allynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂, —NH(lower alkyl), —NH(acyl), —N(lower alkyl)₂, —N(acyl)₂ᵢ (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric forms thereof; or

a pharmaceutically acceptable salt or prodrug thereof,

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system; and

further provided that in Formulae (i)-(ii), Q⁴ and Q⁶ are not simultaneously both N and Q³ and Q⁷ are not C—OH.

In one subembodiment, the method for the treatment of a host infected with a flavivirus, pestivirus or hepacivirus, and in particular HCV, infection comprising administering an effective treatment amount of a compound of Formula (I) or a pharmaceutically acceptable salt or prodrug thereof, is provided wherein:
W is O;
Q¹ is C—R where R is H or halogen;
Q³ is C—R where R is H or halogen, preferably F;
Q⁴ and Q⁶ each independently is N, C—H, or N—H;
Q⁵ is C—R where R is NR⁴R⁵, NHR⁴, or NH₂
Q⁹ and Q¹⁰ each independently is C;
Z is Formula (IV), wherein X is O, S or N—H; R¹, R², and R³ each independently is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); and R⁶ and R¹⁰ each independently is H, alkyl or halo substituted alkyl, Cl, F, Br, or I;

R¹² is optionally H; and

Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, or hydroxyalkyl.

In one subembodiment, the method for the treatment of a host infected with a flavivirus, pestivirus or hepacivirus, and in particular HCV, infection comprising administering an effective treatment amount of a compound of Formula (I) or a pharmaceutically acceptable salt or prodrug thereof, is provided wherein:
W is O;
Q¹ is C—R where R is H;
Q³ is C—R where R is halogen, and preferably F;
Q⁴ and Q⁶ each independently is N;
Q⁵ is C—R where R is NR⁴R⁵, NHR⁴, or NH₂;
Q⁹ and Q¹⁰ each independently is C;
Z is Formula (IV), wherein X is O; R¹, R², R³, R⁸, R¹⁰ and
   R¹¹ each independently is H; and R⁶ is lower alkyl, preferably methyl.

In another subembodiment, the method for the treatment of a host infected with a flavivirus, pestivirus or hepacivirus, and in particular HCV, infection comprising administering an effective treatment amount of a compound of Formula (II) or a pharmaceutically acceptable salt or prodrug thereof, is provided wherein:
W is O;
Q¹ is C—R where R is H;
Q³, Q⁴ and Q⁶ each independently is N or C—R, e.g., C—H;
Q⁷ is C—R where R is NR⁴R⁵, NHR⁴, or NH₂;
Q⁹ and Q¹⁰ each independently is C;
Z is Formula (II), wherein X* is O, S, or C—R where R is H or lower alkyl; R¹ and R² each independently is H, optionally substituted phosphate or phosphonate (including mono-, di-, or triphosphate or a stabilized phosphate prodrug), acyl, alkyl, or amino acid; R⁸ is H, hydroxyl, alkyl, alkenyl, alkynyl, chloro, bromo, fluoro, iodo, or O(alkyl); and R⁷, R⁶ and R¹⁰ is H, alkyl or halo substituted alkyl, Cl, F, Br, or I;

R¹² is optionally H; and

Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, or hydroxyalkyl.

In another subembodiment, the method for the treatment of a host infected with a flavivirus, pestivirus or hepacivirus, and in particular HCV, infection comprising administering an effective treatment amount of a compound of Formula (II) or a pharmaceutically acceptable salt or prodrug thereof, is provided wherein:
W is O;
Q¹ is C—R where R is H;
Q³, Q⁴ and Q⁶ each independently is N;
Q⁷ is C—R where R is NRR, NHR, or NH₂;
Q⁹ and Q¹⁰ each independently is C;
Z is Formula (II), wherein X* is C—R and R is H or lower alkyl; R¹, R², and R⁸ each independently is H; R⁶ is lower alkyl, preferably methyl; and R⁷ is halogen, preferably F.

In another subembodiment, the method for the treatment of a host infected with a flavivirus, pestivirus or hepacivirus, and in particular HCV, infection comprising administering an effective treatment amount of a compound of Formula (Iv) or a pharmaceutically acceptable salt or prodrug thereof, is provided wherein:

W is NR, and R preferably is H;

Q¹, Q⁴, Q⁵, and Q⁶ each independently is C—R where R is H, alkyl, or halogen;

Q³ is N;

Q⁷ each independently is C—R where R is NR⁴R⁵, NHR⁴ or, preferably NH₂;

Q⁹ and Q¹⁹ each independently is C;

Z is Formula (II), wherein X* is N or C—R and R is H or lower alkyl; R¹ and R² each independently is H, optionally substituted phosphate or phosphonate (including mono-, di-, or triphosphate or a stabilized phosphate prodrug), acyl, alkyl, or amino acid; R⁸ is H, hydroxyl, alkyl, alkenyl, alkynyl, chloro, bromo, fluoro, iodo, or O(alkyl); and R⁷, R⁶ and R¹⁰ is H, alkyl or halo substituted alkyl, Cl, F, Br, or I;

R¹² is optionally H; and

Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, or hydroxyalkyl.

In a subembodiment, the method for the treatment of a host infected with a flavivirus, pestivirus or hepacivirus, and in particular HCV, infection comprising administering an effective treatment amount of a compound of Formula (Iv) or a pharmaceutically acceptable salt or prodrug thereof, is provided wherein:
W is NR, and R preferably is H;
Q¹, Q⁴, Q⁵, and Q⁶ each independently is C—R where R is H, alkyl or halogen;
Q³ is N;
Q⁷ each independently is C—R where R is NR⁴R⁵, NHR⁴ or preferably, NH₂;
Q⁹ and Q¹⁹ each independently is C;
Z is Formula (II), wherein X* is C—R and R is H or lower alkyl; R¹, R², R¹⁰ and R⁸ each independently is H; R⁶ is lower alkyl, preferably methyl; and R⁷ is halogen, preferably F.

In a second principal embodiment, a method for the treatment of a host infected with a flavivirus, pestivirus or hepacivirus, and in particular HCV, infection is provided, comprising administering an effective treatment amount of a compound of Formulae (v)-(x):

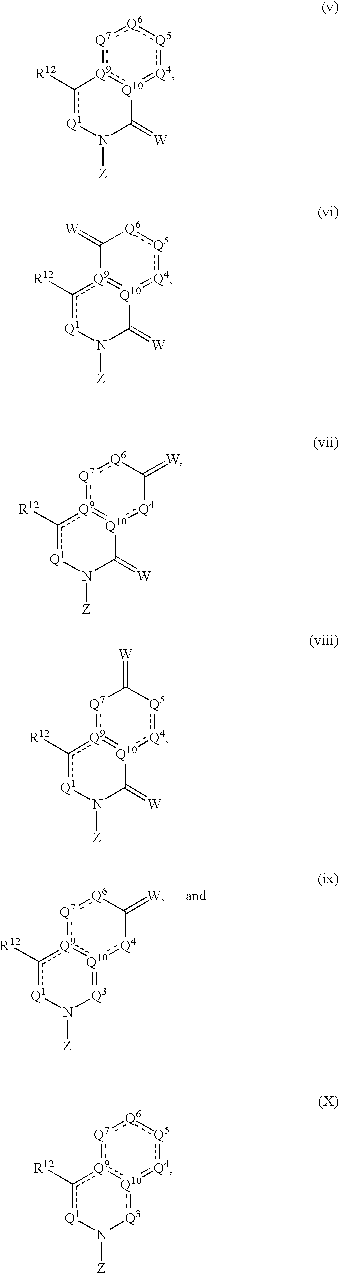

wherein:
Each W is independently O, S or N—R;
Q¹, Q³, Q⁴, Q⁵, Q⁶, Q⁷, Q⁹, and Q¹⁰, each independently, is C—R or N; and
R is each independently H, halo, alkyl, alkenyl, alkynyl, alkoxy, alkoxyalkyl, hydroxyalkyl, cycloalkyl, nitro, cyano, OH, OR⁴, NH₂, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
[symbol] indicates the presence of a single or double bond;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-aryl, C(═O)-alkoxyalkyl, C(═)NH₂, C(═O)NHR⁴, C(═O)N(R⁴)₂, or N₃;
Each Y³ is independently H, F, Cl, Br or I; and
Z is selected from the group consisting of Formulae (I), (II), (III), and (IV):

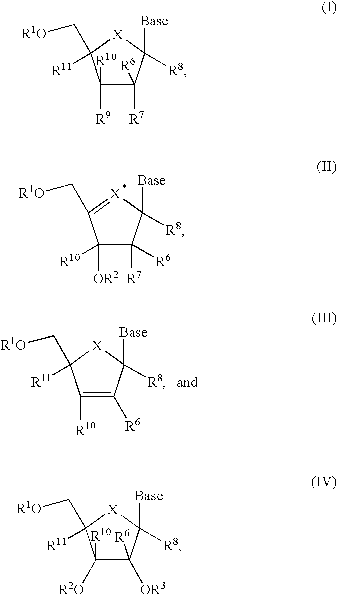

wherein:

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-allynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and each independently is hydrogen, hydroxy, alkyl (including lower alkyl), haloalkyl, haloalkenyl, haloalkynyl, CF₃, N₃, CN, alkenyl, alkynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂; —NH(lower alkyl), —NH(acyl), —N(lower alkyl)₂, —N(acyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric forms thereof; or

a pharmaceutically acceptable salt or prodrug thereof; and

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a subembodiment, the method for the treatment of a host infected with a flavivirus, pestivirus or hepacivirus, and in particular HCV, infection comprising administering an effective treatment amount of a compound of Formula (v) or a pharmaceutically acceptable salt or prodrug thereof, is provided wherein

W is O;

Q¹, Q⁴⁻, Q⁶ and Q⁷ each independently is C—R, e.g. C—H;

Q⁵ is N—R where R is NR⁴R⁵, NHR⁴, or NH₂;

Q⁹ is N;

Q¹⁰ is C;

Z is Formula (IV), wherein X is O, S or N—R where R is H; R², and R³ each independently is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); and R⁶ and R¹⁰ each independently is H, alkyl or halo substituted alkyl, Cl, F, Br, or I;

Each R⁴ and R⁵ independently is H, acyl including lower acyl, alkyl including lower alkyl such as but not limited to methyl, ethyl, propyl and cyclopropyl, alkenyl, allynyl, cycloalkyl, alkoxy, alkoxyalkyl, or hydroxyalkyl;

R¹² is optionally H; and

R is each independently H, halo, alkyl, alkenyl, allynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (v) or a pharmaceutically acceptable salt or prodrug thereof, is provided wherein:

W is O;

Q¹, Q⁴⁻, Q⁶ and Q⁷ each independently is C—R;

Q⁵ is N—R where R is NR⁴R⁵, NHR⁴, or NH₂;

Q⁹ is N;

Q¹⁰ is C;

Z is Formula (IV), wherein X is O; R¹, R², R³, R⁸ and R¹¹ each independently is H; and R⁶ is lower alkyl, preferably methyl.

In another subembodiment, the method for the treatment of a host infected with a flavivirus, pestivirus or hepacivirus, and in particular HCV, infection comprising administering an effective treatment amount of a compound of Formula (vi) or a pharmaceutically acceptable salt or prodrug thereof, is provided wherein:

Each W is O;

Q¹, Q⁴, and Q⁶ each independently is N or C—R;

Q⁵ and Q⁹ each independently is N;

Q¹⁰ is C;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, Cl, F, Br, I, alkyl or halo substituted alkyl, and R⁷ is halogen, OH, H, optionally substituted alkyl, alkenyl or alkynyl, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (vi) or a pharmaceutically acceptable salt or prodrug thereof, is provided wherein:

Each W is O;

Q¹, Q⁴, and Q⁶ each independently is C—R;

Q⁵ and Q⁹ each independently is N;

Q¹⁰ is C;

Z is Formula (I), wherein X is NH; R¹, R⁸, R¹⁰ and R¹¹ each independently is H; R⁶ is lower alkyl, preferably methyl; and R⁷ and R⁹ each independently is OH.

In yet another subembodiment, the method for the treatment of a host infected with a flavivirus, pestivirus or hepacivirus, and in particular HCV, infection comprising administering an effective treatment amount of a compound of Formula (vii) or a pharmaceutically acceptable salt or prodrug thereof, is provided wherein:

Each W is O;

Q¹, Q⁴, Q⁶, and Q⁷ each independently is C—R;

Q⁹ is N;

Q¹⁰ is C;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, OH, Cl, F, Br, I, optionally substituted alkyl, alkenyl or alkynyl, alkoxy, CH₂OH, or hydroxyalkyl; and R⁷ is halogen, OH, H, optionally substituted alkyl, alkenyl or alkynyl, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (vii) or a pharmaceutically acceptable salt or prodrug thereof, is provided wherein: Each W is O;

Q¹, Q⁴, Q⁶, and Q⁷ each independently is C—R;

Q⁹ is N;

Q¹⁰ is C;

Z is Formula (I), wherein X is O; R¹, R⁸, R¹⁰ and R¹¹ (each independently is H;

R⁶ is lower alkyl, preferably methyl;

R⁷ is halogen, preferably F; and

R⁹ is OH.

In still another subembodiment, the method for the treatment of a host infected with a flavivirus, pestivirus or hepacivirus, and in particular HCV, infection comprising administering an effective treatment amount of a compound of Formula (viii) or a pharmaceutically acceptable salt or prodrug thereof, is provided wherein:

Each W is N—R;

Q¹, Q⁴, Q⁵, and Q⁷ each independently is C—R;

Q⁹ is N;

Q¹⁰ is C;

Z is Formula (III), wherein X is O, S or N—R where R is H;

R¹ is H, optionally substituted phosphate or phosphonate (including mono-, di-, or triphosphate or a stabilized phosphate prodrug), acyl, alkyl, or amino acid; R¹⁰ and R⁶ is H, alkyl or halo substituted alkyl, chloro, bromo, fluoro, or iodo;

R⁸ and R¹¹ each independently is H, hydroxyl, alkyl, alkenyl, alkynyl, chloro, bromo, fluoro, iodo, or O(alkyl);

R¹² is optionally H; and

R is each independently H, halo, alkyl, alkenyl, allynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(O)NH₂, C(═O)NHR⁴, C(═O)N(R⁴)₂, or N₃.

In still another subembodiment, the method for the treatment of a host infected with a flavivirus, pestivirus or hepacivirus, and in particular HCV, infection comprising administering an effective treatment amount of a compound of Formula (viii) or a pharmaceutically acceptable salt or prodrug thereof, is provided wherein:

Each W is N—R;

Q¹, Q⁴, Q⁵ and Q⁷ each independently is C—R;

Q⁹ is N;

Q¹⁰ is C;

Z is Formula (II), wherein X is O;

R¹, R¹⁰, and R¹¹ each independently is H;

R⁸ is alkyl; and

R⁶ is lower alkyl, preferably methyl.

In another subembodiment, the method for the treatment of a host infected with a flavivirus, pestivirus or hepacivirus, and in particular HCV, infection comprising administering an effective treatment amount of a compound of Formula (ix) or a pharmaceutically acceptable salt or prodrug thereof, is provided wherein:

Q¹, Q³, Q⁴, Q⁶, and Q⁷ each independently is C—R;

Q⁹ is N;

Q¹⁰ is C;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, Cl, F, Br, I, alkyl or halo substituted alkyl; and R⁷ is halogen, OH, H, optionally substituted alkyl, alkenyl or alkynyl, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (ix) or a pharmaceutically acceptable salt or prodrug thereof, is provided wherein:

Q¹, Q³, Q⁴, Q⁶, and Q⁷ each independently is C—R;

Q⁹ is N;

Q¹⁰ is C;

Z is Formula (I), wherein X is S;

R¹, R⁸, R¹⁹, and R¹¹ each independently is H;

R⁶ is lower alkyl, preferably methyl

R⁹ is OH; and

R⁷ is halogen, preferably F.

In yet another subembodiment, the method for the treatment of a host infected with a flavivirus, pestivirus or hepacivirus, and in particular HCV, infection comprising administering an effective treatment amount of a compound of Formula (x) or a pharmaceutically acceptable salt or prodrug thereof, is provided wherein:

Q¹, Q³, Q⁴, Q⁵, Q⁶, and Q⁷ each independently is C—R;

Q⁹ is N;

Q¹⁰ is C;

Z is Formula (II), wherein X* is O, S, NH, or C—R and R is H or lower alkyl; R¹ and R² each independently is H, optionally substituted phosphate or phosphonate (including mono-, di-, or triphosphate or a stabilized phosphate prodrug), acyl, alkyl, or amino acid; R⁸ is H, hydroxyl, alkyl, alkenyl, alkynyl, chloro, bromo, fluoro, iodo, or O(alkyl); and R⁷, R⁶ and R¹⁰ is H, OH, optionally substituted alkyl, alkenyl, or alkynyl, Cl, F, Br, I, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In yet another subembodiment, the method for the treatment of a host infected with a flavivirus, pestivirus or hepacivirus, and in particular HCV, infection comprising administering an effective treatment amount of a compound of Formula (x) or a pharmaceutically acceptable salt or prodrug thereof, is provided wherein:

Q¹, Q³, Q⁴, Q⁵, Q⁶, and Q⁷ each independently is C—R;

Q⁹ is N;

Q¹⁰ is C;

Z is Formula (II), wherein X* is C—R and R is H or lower alkyl;

R¹, R², R⁸, and R¹⁹ each independently is H;

R⁶ is lower alkyl, preferably methyl; and

R⁷ is halogen, preferably F.

In a third principal embodiment, a method for the treatment of a host infected with a flavivirus, pestivirus or hepacivirus, and in particular HCV, infection is provided, comprising administering an effective treatment amount of a compound of Formulae (xi)-(xiii):

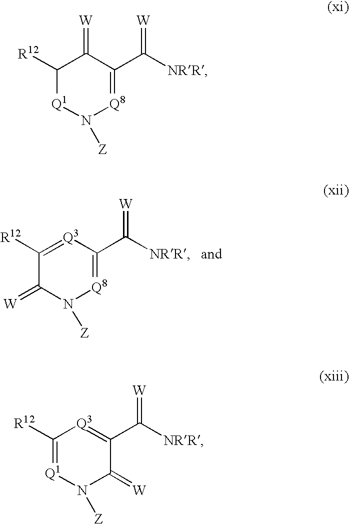

wherein,
Each W is independently O, S or N—R;
Q¹, Q³, and Q⁸, each independently, is C—R or N; and
R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(O)OH, C(═O)OR⁴, C(═O)-alkyl, CO)-aryl, C(═O)-alkoxyalkyl, C(O)NH₂, C(═O)NHR⁴, C(═O)N(R⁴)₂, or N₃;
R′ is each independently H, halo, alkyl, alkenyl; alkynyl, alkoxy, alkoxyalkyl, hydroxyalkyl, cycloalkyl, nitro, cyano, OH, OR⁴, NH₂, NHR⁴, NR⁴R⁵; SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
Each Y³ is independently H, F, Cl, Br or I; and
Z is selected from the group consisting of Formulae (I), (II), (III), and (IV):

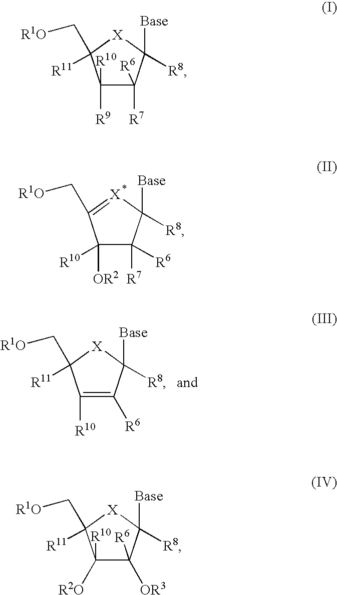

wherein:

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkenyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-allynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, alkynyl, Br-vinyl, C(Y³)₃; C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower —O(alkenyl), chloro, bromo, fluoro, iodo, CH₂F, CH₂Cl, CH₂CF₃, CF₂CF₃, NO₂, NH₂, —NH(lower alkyl), —NH(acyl), —N(lower alkyl)₂, —N(acyl)₂, (CH₂)ₘC(O)OH, (CH₂)ₘC(O)OR⁴, (CH₂)ₘC(O)O(lower alkyl), (CH₂)ₘC(O)NH₂; (CH₂)ₘC(O)NHR⁴, (CH₂)ₘC(O)NH(lower (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric forms thereof; or

a pharmaceutically acceptable salt or prodrug thereof;

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a subembodiment, the method for the treatment of a host infected with a flavivirus, pestivirus or hepacivirus, and in particular HCV, infection comprising administering an effective treatment amount of a compound of Formula (xi) or a pharmaceutically acceptable salt or prodrug thereof, is provided wherein:

Each W is independently O or N—R;

Q¹, and Q⁸ each independently is C—R;

Z is Formula (IV), wherein X is O, S or N—R where R is H; R¹, R², and R³ each independently is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); and R⁶ and R¹⁰ each independently is H, alkyl or halo substituted alkyl, Cl, F, Br, or I;

R¹² is optionally H;

R′ is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xi) or a pharmaceutically acceptable salt or prodrug thereof, is provided wherein:

Each W is independently O or N—R;

Q¹ and Q⁸ each independently is C—R;

Z is Formula (IV), wherein X is O; R¹, R², R³, R¹⁰, and R¹¹ each independently is H; and

R⁶ and R⁸ is lower alkyl, preferably methyl or ethyl.

In another subembodiment, the method for the treatment of a host infected with a flavivirus, pestivirus or hepacivirus, and in particular HCV, infection comprising administering an effective treatment amount of a compound of Formula (xii) or a pharmaceutically acceptable salt or prodrug thereof, is provided wherein:

Each W is independently O or NH;

Q³ and Q⁸ each independently is C—R;

Z is Formula (II), wherein X* is O, S, or N or C—R and R is H or lower alkyl; R¹ and R² each independently is H, optionally substituted phosphate or phosphonate (including mono-, di-, or triphosphate or a stabilized phosphate prodrug), acyl, alkyl, or amino acid; R⁸ is H, hydroxyl, alkyl, alkenyl, alkynyl, chloro, bromo, fluoro, iodo, or O(alkyl); and R⁷, R⁶ and R¹⁰ is H, OH, optionally substituted alkyl, alkenyl, or alkynyl, Cl, F, Br, I, alkoxy, CH₂OH, or hydroxyalkyl;

R′ is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, Mlle, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;

In a subembodiment, the method for the treatment of a host infected with a flavivirus, pestivirus or hepacivirus, and in particular HCV, infection comprising administering an effective treatment amount of a compound of Formula (xii) or a pharmaceutically acceptable salt or prodrug thereof, is provided wherein:

W is O in both instances;

Q³ and Q⁸ each independently is C—R;

Z is Formula (II), wherein X is N;

R¹, R², R⁸, and R¹⁰ each independently is H;

R⁶ is lower alkyl, preferably methyl; and

R⁷ is halo, preferably F.

In yet another subembodiment, the method for the treatment of a host infected with a flavivirus, pestivirus or hepacivirus, and in particular HCV, infection comprising administering an effective treatment amount of a compound of Formula (xiii) or a pharmaceutically acceptable salt or prodrug thereof, is provided wherein:

Each W is independently O or N—R, e.g. NH;

Q¹ and Q³ each independently is N or C—R where R is H or halogen;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, OH, Cl, F, Br, I, optionally substituted alkyl, alkenyl or alkynyl, alkoxy, CH₂OH, or hydroxyalkyl; and R⁷ is halogen, OH, H, optionally substituted alkyl, alkenyl or alkynyl, alkoxy, CH₂OH, or hydroxyalkyl; and

R¹² is optionally H.

In a subembodiment, the method for the treatment of a host infected with a flavivirus, pestivirus or hepacivirus, and in particular HCV, infection comprising administering an effective treatment amount of a compound of Formula (xiii) or a pharmaceutically acceptable salt or prodrug thereof, is provided wherein:

Each W is independently O or N—R;

Q¹ and Q³ each independently is N;

Z is Formula (I), wherein X is O;

R¹, R⁸, R¹⁰, and R¹¹ each independently is H; and

R⁶ is lower alkyl, preferably methyl; and

R⁷ is OH or halo, preferably F; and

R⁹ is OH.

In a fourth principal embodiment, a method for the treatment of a host infected with a flavivirus, pestivirus or hepacivirus, and in particular HCV, infection is provided, comprising administering an effective treatment amount of a compound of base Formulae (xiv)-(xviii):

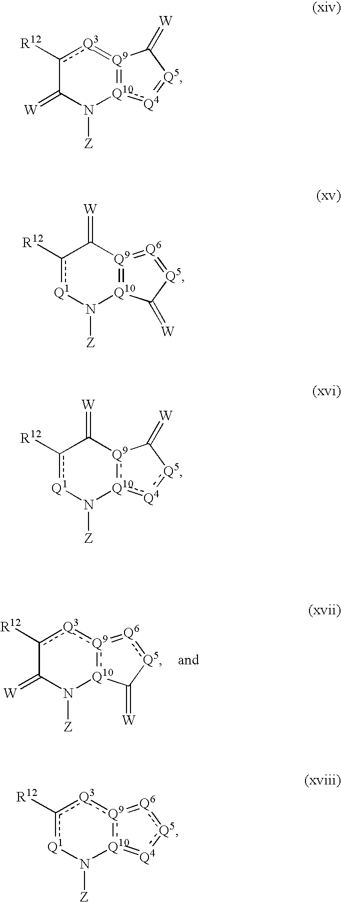

wherein:
Each W is independently O, S or N—R;
Q¹, Q³, Q⁴, Q⁵, Q⁶, Q⁹, and Q¹⁰, each independently, is C—R or N; and
R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(O)OR⁴, C(═O)-alkyl, C(═O)-aryl, C(═O)-alkoxyalkyl, C(═O)NH₂, C(═O)NHR⁴, C(═O)N(R⁴)₂, or N₃;
[symbol] indicates the presence of a single or double bond;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)³, C(Y³)₂C(Y³)₃, C(═O)OH, C(═O)OR⁴, C(═O)-alkyl, C(═O)-aryl, C(═O)-alkoxyalkyl, C(═)NH₂, C(═O)NHR⁴, C(═O)N(R⁴)₂, or N₃;
Each Y³ is independently H, F, Cl, Br or I; and
Z is selected from the group consisting of Formulae (I), (II), (III), and (IV):

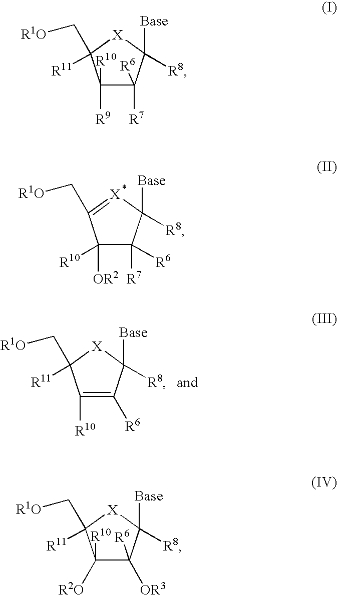

wherein:

R¹, R², R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or allynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, allynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂, —NH(lower alkyl), —NH(acyl), —N(lower alkyl)₂, —N(acyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric forms thereof; or

a pharmaceutically acceptable salt or prodrug thereof;

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system; and

further provided that in Formula (xviii) Q⁵ and Q⁶ are not simultaneously both N or N—R.

In a subembodiment, the method for the treatment of a host infected with a flavivirus, pestivirus or hepacivirus, and in particular HCV, infection comprising administering an effective treatment amount of a compound of Formula (xiv) or a pharmaceutically acceptable salt or prodrug thereof, is provided wherein:

Each W is O;

Q⁴ is C—R;

Q³ and Q⁵ each independently is N—R;

Q⁹ and Q¹⁰ each independently is C;

Z is Formula (IV), wherein X is O, S or N—R where R is H; R¹, R², and R³ each independently is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); and R⁶ and R¹⁰ each independently is H, alkyl or halo substituted alkyl, Cl, F, Br, or I;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xiv) or a pharmaceutically acceptable salt or prodrug thereof, is provided wherein:

W is O;

Q⁴ is C—R;

Q³ and Q⁵ each independently is N—R;

Q⁹ and Q¹⁰ each independently is C;

Z is Formula (IV), wherein X is O; R¹, R², R³, and R⁸ each independently is H; R¹⁰ and R¹¹ each independently is H or lower alkyl; and R⁶ is lower alkyl, preferably methyl.

In another subembodiment, the method for the treatment of a host infected with a flavivirus, pestivirus or hepacivirus, and in particular HCV, infection comprising administering an effective treatment amount of a compound of Formula (xv) or a pharmaceutically acceptable salt or prodrug thereof, is provided wherein:

Each W is independently O or N—R;

Q¹, Q⁵ and Q⁶ each independently is C—R;

Q⁹ and Q¹⁰ each independently is C;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid;

R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, OH, Cl, F, Br, I, optionally substituted alkyl, alkenyl or alkynyl, alkoxy, CH₂OH, or hydroxyalkyl; and R⁷ is halogen, OH, H, optionally substituted alkyl, alkenyl or alkynyl, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xv) or a pharmaceutically acceptable salt or prodrug thereof, is provided wherein:

Each W is independently O or N—R;

Q¹, Q⁵ and Q⁶ each independently is C—R;

Q⁹ and Q¹⁰ each independently is C;

Z is Formula (I), wherein X is O;

R⁷ and R⁹ each independently is OH;

R¹, R⁸ and R¹⁰ each independently is H;

R¹¹ is H or lower alkyl; and

R⁶ is lower alkyl, preferably methyl.

In another subembodiment, the method for the treatment of a host infected with a flavivirus, pestivirus or hepacivirus, and in particular HCV, infection comprising administering an effective treatment amount of a compound of Formula (xvi) or a pharmaceutically acceptable salt or prodrug thereof, is provided wherein:

Each W is independently O or N—R;

Q¹ and Q⁴ each independently is C—R;

Q⁵ is N—R;

Q⁹ and Q¹⁰ each independently is C;

Z is Formula (II), wherein X* is C—R⁴ or CF; R¹ and R² each independently is H, optionally substituted phosphate or phosphonate (including mono-, di-, or triphosphate or a stabilized phosphate prodrug), acyl, alkyl, or amino acid; R⁸ is H, hydroxyl, alkyl, alkenyl, alkynyl, chloro, bromo, fluoro, iodo, or O(alkyl); and R⁷, R⁶ and R¹⁰ is H, OH, optionally substituted alkyl, alkenyl, or alkynyl, Cl, F, Br, I, alkoxy, CH₂OH, or hydroxyalkyl;

R¹² is optionally H;

R⁴ is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xvi) or a pharmaceutically acceptable salt or prodrug thereof, is provided wherein:

Each W is independently O or N—R;

Q¹ and Q⁴ each independently is C—R;

Q⁵ is N—R;

Q⁹ and Q¹⁰ each independently is C;

Z is Formula (II), wherein X* is C—R⁴ or CF; R¹, R² and R⁸ each independently is H; R¹⁰ is H, alkyl or alkenyl;

R⁶ is lower alkyl, preferably methyl; and

R⁷ is OH or halo.

In another subembodiment, the method for the treatment of a host infected with a flavivirus, pestivirus or hepacivirus, and in particular HCV, infection comprising administering an effective treatment amount of a compound of Formula (xvii) or a pharmaceutically acceptable salt or prodrug thereof, is provided wherein:

Each W is independently O or N—R;

Q³, Q⁵ and Q⁶ each independently is N or C—R;

Q⁹ and Q¹⁰ each independently is C;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, OH, Cl, F, Br, I, optionally substituted alkyl, alkenyl or alkynyl, alkoxy, CH₂OH, or hydroxyalkyl; and R⁷ is halogen, OH, H, optionally substituted alkyl, alkenyl or allynyl, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xvii) or a pharmaceutically acceptable salt or prodrug thereof, is provided wherein:

Each W is independently O or N—R;

Q³, Q⁵ and Q⁶ each independently is C—R;

Q⁹ and Q¹⁰ each independently is C;

Z is Formula (I), wherein X is S; R¹, R⁸ and R¹⁰ each independently is H; R⁷ is OH or halo, preferably F; R⁹ is OH; R¹¹ is H or lower alkyl; and R⁶ is lower alkyl, preferably methyl.

In another subembodiment, the method for the treatment of a host infected with a flavivirus, pestivirus or hepacivirus, and in particular HCV, infection comprising administering an effective treatment amount of a compound of Formula (xviii) or a pharmaceutically acceptable salt or prodrug thereof, is provided wherein:

Q¹, Q⁴ and Q⁶ each independently is C—R or N;

Q³ and Q⁵ each independently is C—R or N;

Q⁹ and Q¹⁰ each independently is C;

Z is Formula (n), wherein X is O, S or N—R where R is H;

R¹ is H, optionally substituted phosphate or phosphonate (including mono-, di-, or triphosphate or a stabilized phosphate prodrug), acyl, alkyl, or amino acid; R⁶ and R¹⁰ is H, alkyl or halo substituted alkyl, chloro, bromo, fluoro, or iodo,

R⁸ and R¹¹ each independently is H, OH alkyl, alkenyl, alkynyl, chloro, bromo, fluoro, iodo, or O(alkyl);

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In another subembodiment, the method for the treatment of a host infected with a flavivirus, pestivirus or hepacivirus, and in particular HCV, infection comprising administering an effective treatment amount of a compound of Formula (xviii) or a pharmaceutically acceptable salt or prodrug thereof, is provided wherein:

Q¹, Q⁴ and Q⁶ each independently is C—R;

Q³ and Q⁵ each independently is N;

Q⁹ and Q¹⁰ each independently is C;

Z is Formula (III), wherein X is O;

R¹ is H;

R⁸ and R¹¹ each independently is H or lower alkyl;

R⁶ is lower alkyl, preferably methyl; and

R¹⁰ is H or alkyl.

In a fifth principal embodiment, a method for the treatment of a host infected with a flavivirus, pestivirus or hepacivirus, and in particular HCV, infection is provided, comprising administering an effective treatment amount of a compound of base Formulae (xix)-(xxii):

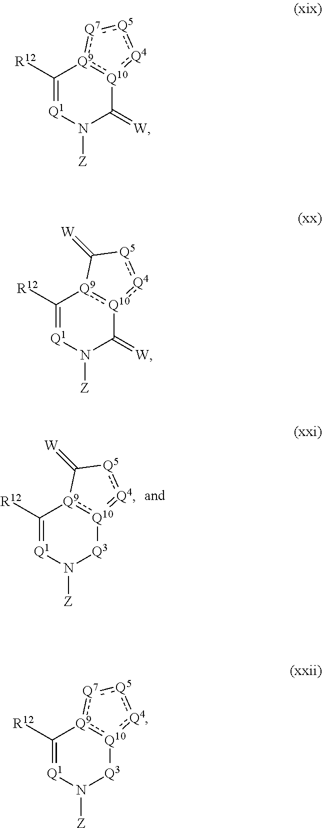

wherein:
W is each independently O, S or N—R;
Q¹, Q³, Q⁴, Q⁵, Q⁷, Q⁹, and Q¹⁰, each independently, is C—R or N;
R is each independently H, halo, alkyl, alkenyl, alkynyl, alkoxy, alkoxyalkyl, hydroxyalkyl, cycloalkyl, nitro, cyano, OH, OR⁴, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-alkoxyalkyl, C(═O)NH₂, C(═O)NHR⁴, C(O)N(R⁴)₂, or N₃;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
Y³ is each independently H, F, Cl, Br or I;
[symbol] indicates the presence of a single or a double bond; and
Z is selected from the group consisting of Formulae (I), (II), (III), and (IV):

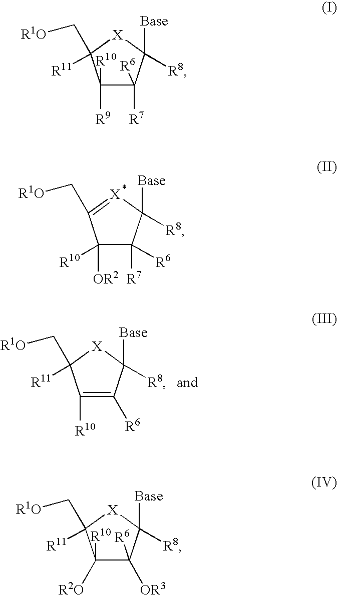

wherein:

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, (CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, allynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂, —NH(lower alkyl), —NH(acyl), —N(lower alkyl)₂, —N(acyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric forms thereof; or

a pharmaceutically acceptable salt or prodrug thereof;

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a subembodiment, the method for the treatment of a host infected with a flavivirus, pestivirus or hepacivirus, and in particular HCV, infection comprising administering an effective treatment amount of a compound of Formula (xix) or a pharmaceutically acceptable salt or prodrug thereof, is provided wherein:

W is O;

Q¹, Q⁴, Q⁵ and Q⁷ each independently is C—R or N;

Q⁹ is N;

Q¹⁰ is C;

Z is Formula (I), wherein X is O, S or N—R where R is H; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, OH, Cl, F, Br, I, optionally substituted alkyl, alkenyl or alkynyl, alkoxy, CH₂OH, or hydroxyalkyl; and R⁷ is halogen, OH, H, optionally substituted alkyl, alkenyl or alkynyl, alkoxy, CH₂OH, or hydroxyalkyl;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xix) or a pharmaceutically acceptable salt or prodrug thereof, is provided wherein:

W is O;

Q¹, Q⁴, Q⁷ each independently is C—R;

Q⁹ is N;

Q¹⁰ is C;

Z is Formula (I), wherein X is O; R¹, R⁸, R¹⁰ and R¹¹ each independently is H; R⁶ is lower alkyl, preferably methyl; R⁹ is OH; R⁷ is OH or halo, preferably F.

In another subembodiment, the method for the treatment of a host infected with a flavivirus, pestivirus or hepacivirus, and in particular HCV, infection comprising administering an effective treatment amount of a compound of Formula (xx) or a pharmaceutically acceptable salt or prodrug thereof, is provided wherein:

Each W is O;

Q¹, and Q⁴ each independently is C—R;

Q⁵ is N—H;

Q⁹ is N;

Q¹⁰ is C;

Z is Formula (II), wherein X* is O, S, C—R⁴ or CF; R¹ and R² each independently is H, optionally substituted phosphate or phosphonate (including mono-, di-, or triphosphate or a stabilized phosphate prodrug), acyl, alkyl, or amino acid; R⁸ is H, hydroxyl, alkyl, alkenyl, alkynyl, chloro, bromo, fluoro, iodo, or O(alkyl); and R⁷, R⁶ and R¹⁰ is H, OH, optionally substituted alkyl, alkenyl, or alkynyl, Cl, F, Br, I, alkoxy, CH₂OH, or hydroxyalkyl;

R¹² is optionally H;

R⁴ is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xx) or a pharmaceutically acceptable salt or prodrug thereof, is provided wherein:

W is O;

Q¹, Q², and Q⁴ each independently is C—R;

Q⁵ is N—H;

Q⁹ is N;

Q¹⁰ is C;

Z is Formula (II), wherein X* is CY³ or C—R⁴; R¹, R², R⁸ and R¹⁰ each independently is H;

R⁶ is lower alkyl, preferably methyl; and

R⁷ is OH or halo, preferably F.

In yet another subembodiment, the method for the treatment of a host infected with a flavivirus, pestivirus or hepacivirus, and in particular HCV, infection comprising administering an effective treatment amount of a compound of Formula (xxi) or a pharmaceutically acceptable salt or prodrug thereof, is provided wherein:

W is O;

Q¹, Q³ and Q⁴ each independently is N or C—R;

Q⁵ and Q⁹ each independently is N;

Q¹⁰ is C;

Z is Formula (III), wherein X is O, S or N—R;

R¹ is H, optionally substituted phosphate or phosphonate (including mono-, di-, or triphosphate or a stabilized phosphate prodrug), acyl, alkyl, or amino acid; R⁶ and R¹⁰ is H, alkyl or halo substituted alkyl, chloro, bromo, fluoro, or iodo;

R⁸ and R¹¹ each independently is H, hydroxyl, alkyl, alkenyl, alkynyl, chloro, bromo, fluoro, iodo, or O(alkyl);

R¹² is optionally H; and

R is each independently H, halo, alkyl, alkenyl, allynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivinis, and in particular HCV, infection comprising administering an effective treatment amount of a compound of Formula (xxi) or a pharmaceutically acceptable salt or prodrug thereof, is provided wherein:

W is O;

Q¹, Q³ and Q⁴ each independently is C—R;

Q⁵ and Q⁹ each independently is N;

Q¹⁰ is C;

Z is Formula (III), wherein X is O or N—R; R¹ is H; R⁶ is CN, N₃, or lower alkyl, preferably methyl; R⁸ and R¹¹ each independently is H or alkyl; and R¹⁰ is H or CF₃.

In still another subembodiment, the method for the treatment of a host infected with a flavivirus, pestivirus or hepacivirus, and in particular HCV, infection comprising administering an effective treatment amount of a compound of Formula (xxii) or a pharmaceutically acceptable salt or prodrug thereof, is provided wherein:

Q¹, Q³, Q⁵ and Q⁷ each independently is C—R or N;

Q⁴ and Q⁹ each independently is N;

Q¹⁰ is C;

Z is Formula (IV), wherein X is O, S or N—R where R is H; R¹, R², and R³ each independently is H, optionally substituted phosphate or phosphonate (including mono-, di-, or triphosphate or a stabilized phosphate, prodrug), acyl, alkyl, or amino acid; R⁸ and R¹¹ each independently is H, hydroxyl, alkyl, alkenyl, alkynyl, chloro, bromo, fluoro, iodo, or O(alkyl); and R⁶ and R¹⁰ each independently is H, alkyl or halo substituted alkyl, Cl, F, Br, or I;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, Nine, NR⁴R⁵, SH, SR⁴, CF₃, CH₂OH, CH₂F, CH₂Cl, CH₂CF₃, C(Y³)₂C(Y³)₃, C(═O)OH, C(O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xxii) or a pharmaceutically acceptable salt or prodrug thereof, is provided wherein:

Q¹, Q³, Q⁵ and Q⁷ each independently is C—R or N;

Q⁴ and Q⁹ each independently is N;

Q¹⁰ is C;

Z is Formula (IV), wherein X is O; R¹, R², R⁸, R¹⁰ and R¹¹ each independently is H;

R³ is H or lower alkyl; and

R⁶ is lower alkyl, preferably methyl.

In a sixth principal embodiment, a method for the treatment of a host infected with a flavivirus, pestivirus or hepacivirus, and in particular HCV, infection is provided, comprising administering an effective treatment amount of a compound of base Formulae (xxiii)-(xxiv):

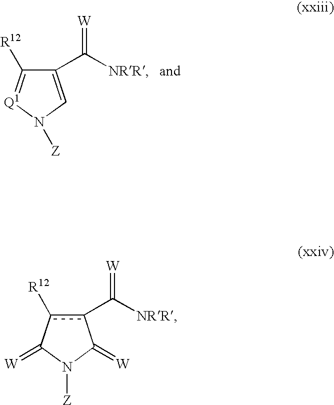

wherein:
W is each independently O, S or N—R;
Q¹ is C—R or N; and
R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
R′ is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
Each R⁴ and R⁵ independently is H, acyl including lower acyl, alkyl including lower alkyl such as but not limited to methyl, ethyl, propyl and cyclopropyl, alkenyl, alkynyl, cycloalkyl, alkoxy, alkoxyalkyl, hydroxyalkyl, or aryl;
R¹² is H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃;
Y³ is each independently H, F, Cl, Br or I; and
Z is selected from the group consisting of Formulae (I), (II), (III), and (IV):

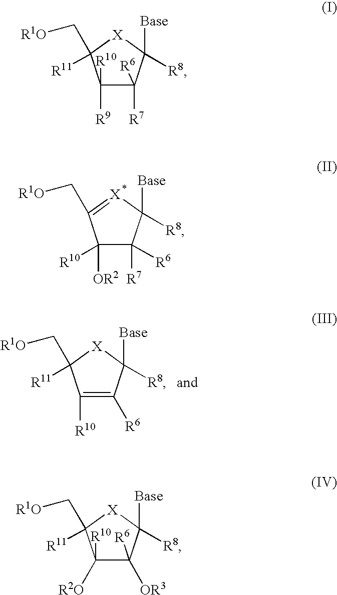

wherein:

R¹, R², and R³, each independently, is hydrogen, optionally substituted phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol; or other pharmaceutically acceptable leaving group that, in vivo, provides a compound wherein R¹ is independently H or mono-, di- or tri-phosphate;

R⁶ and R¹⁰ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), —N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, CH₂C(O)SH, CH₂C(O)SR⁴, CH₂C(O)S(lower alkyl), or C₃₋₇ cycloalkylamino;

R⁷ and R⁹ each independently is H, OH, SH, NH₂, NHR, NR⁴R⁵, CF₃, Cl, F, Br, I, F, optionally substituted alkyl, optionally substituted alkenyl or alkynyl, haloalkenyl, haloalkynyl, Br-vinyl, —CH₂OH, alkoxy, alkoxyalkyl, hydroxyalkyl, CH₂F, CH₂N₃, CH₂CN, CF₂CF₃, (CH₂)ₘC(O)OR⁴, CN, N₃, NO₂, C(Y³)₃, OCN, NCO, 2-Br-ethyl, CH₂Cl, CH₂CF₃, C(═O)-alkyl, O-acyl, O-alkyl, O-alkenyl, O-alkynyl, O-aralkyl, O-cycloalkyl, C(═O)O-alkyl, CH₂NH₂, CH₂NHCH₃, CH₂N(CH₃)₂, —(CH₂)ₘC(O)NHR⁴, CH₂C(O)OH, (CH₂)ₘC(O)N(R⁴)₂, CH₂C(O)OR⁴, CH₂C(O)O(lower alkyl), CH₂C(O)NH₂, CH₂C(O)NHR⁴, CH₂C(O)NH(lower alkyl), CH₂C(O)N(R⁴)₂, CH₂C(O)N(lower alkyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, C(═O)OH, C(═O)OR⁴, C(═O)O(lower alkyl), C(═O)NH₂, C(O)NHR⁴, C(O)NH(lower alkyl), C(O)N(R⁴)₂, —NH(alkyl), N(alkyl)₂, —NH(acyl), —N(acyl)₂, C(Y³)₂C(Y³)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or C₃₋₇ cycloalkylamino;

R⁸ and R¹¹ each independently is hydrogen, hydroxy, alkyl (including lower alkyl), haloalkyl, haloalkenyl, haloalkynyl, CF₃, N₃, CN, alkenyl, alkynyl, Br-vinyl, C(Y³)₃, C(Y³)₂C(Y³)₂, OCN, NCO, 2-Br-ethyl, —C(O)O(alkyl), —C(O)OH, —O(acyl), —O(lower acyl), —O(alkyl), CH₂CN, CH₂N₃, CH₂NH₂, CH₂N(CH₃)₂, CH₂NHCH₃, O(lower alkyl), —O(alkenyl), chloro, bromo, fluoro, iodo, CH₂F, CH₂Cl, CH₂CF₃, CF₂CF₃, NO₂, NH₂, —NH(lower alkyl), —NH(acyl), —N(lower alkyl)₂, —N(acyl)₂, (CH₂)ₘC(O)OH, (CH₂)ₘC(O)OR⁴, (CH₂)ₘC(O)O(lower alkyl), (CH₂)ₘC(O)NH₂, (CH₂)ₘC(O)NHR⁴, (CH₂)ₘC(O)NH(lower alkyl), (CH₂)ₘC(O)N(R⁴)₂, (CH₂)ₘC(O)N(lower alkyl)₂, SR⁴, —S-alkyl, S-alkenyl, S-alkynyl, S-acyl, S-aralkyl, S-cycloalkyl, (CH₂)ₘC(O)SH, (CH₂)ₘC(O)SR⁴, CH₂C(O)S(lower alkyl), or cycloalkylamino;

X is O, S, N—R, SO₂ or CH₂;

X* is CH, N, CF, CY³ or C—R⁴;

m is 0, 1 or 2;

all tautomers, stereoisomers and enantiomeric farms thereof; or

a pharmaceutically acceptable salt or prodrug thereof;

provided that the bicyclic ring system in any of Formulae (i)-(ii), (iv)-(x) and (xiv)-(xxii) comprises no more than 5 nitrogen atoms in the bicyclic ring and no more than 3 nitrogen atoms in any single ring of the bicyclic ring system.

In a subembodiment, the method for the treatment of a host infected with a flavivirus, pestivirus or hepacivirus, and in particular HCV, infection comprising administering an effective treatment amount of a compound of Formula (xxiii) or a pharmaceutically acceptable salt or prodrug thereof, is provided wherein:

W is O or N—R;

Q¹ is C—R;

Z is Formula (I), wherein X is O, S or N—R; R¹ is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); R⁶, R⁹, and R¹⁰ each independently is H, OH, Cl, F, Br, I, optionally substituted alkyl, alkenyl or alkynyl, alkoxy, CH₂OH, or hydroxyalkyl; and R⁷ is halogen, OH, H, optionally substituted alkyl, alkenyl or alkynyl, alkoxy, CH₂OH, or hydroxyalkyl;

R¹² is optionally H;

R′ is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═)NH₂, C(Y³)NHR⁴, C(═O)N(R⁴)₂, or N₃;

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xxiii) or a pharmaceutically acceptable salt or prodrug thereof, is provided wherein:

W is O or N—R;

Q¹ is C—R;

Z is Formula (I), wherein X is O or N—R; R¹, R⁸, R¹⁰ and R¹¹ each independently is H;

R⁷ and R⁹ each independently is OH; and

R⁶ is lower alkyl, preferably methyl.

In a subembodiment, the method for the treatment of a host infected with a flavivirus, pestivirus or hepacivirus, and in particular HCV, infection comprising administering an effective treatment amount of a compound of Formula (xxiv) or a pharmaceutically acceptable salt or prodrug thereof, is provided wherein:

Each W is independently O or N—R;

Z is Formula (IV), wherein X is O, S or N—R where R is H; R¹, R², and R³ each independently is H, optionally substituted phosphate or phosphonate (including mono-, di-, or triphosphate or a stabilized phosphate prodrug), acyl, alkyl, or amino acid; R⁸ and R¹¹ each independently is H, hydroxyl, alkyl, alkenyl, alkynyl, chloro, bromo, fluoro, iodo, or O(alkyl); and R⁶ and R¹⁰ each independently is H, alkyl or halo substituted alkyl, Cl, F, Br, or I;

R′ is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═)NH₂, C(═O)NHR⁴, C(═O)N(R⁴)₂, or N₃;

R¹² is optionally H; and

R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃.

In a subembodiment, the method for the treatment of a host infected with a flavivirus, pestivirus or hepacivirus, and in particular HCV, infection comprising administering an effective treatment amount of a compound of Formula (xxiv) or a pharmaceutically acceptable salt or prodrug thereof, is provided wherein:

W is O or N—R;

Z is Formula (IV), wherein X is O or S;

R1, R² and R¹⁰ each independently is H;

R⁸ and R¹¹ each independently is H or alkyl;

R⁶ is lower alkyl, preferably methyl; and

R³ is H or alkyl.

The β-D- and β-L-nucleosides of this invention inhibit flavivirus, pestivirus or hepacivirus enzymatic activity. Nucleosides can be screened for their ability to inhibit flavivirus, pestivirus or hepacivirus enzyme activity in vitro according to screening methods set forth more particularly herein. One can readily determine the spectrum of activity by evaluating the compound in the assays described herein or with another confirmatory assay.

In one embodiment the efficacy of the anti-flavivirus, pestivirus or hepacivirus compound is measured according to the concentration of compound necessary to reduce the plaque number of the virus in vitro, according to methods set forth more particularly herein, by 50% (i.e. the compound's EC₅₀). In preferred embodiments the compound exhibits an EC_{S}' of less than 15 or preferably, less than 10 micromolar in vitro.

The active compound can be administered as any salt or prodrug that upon administration to the recipient directly or indirectly provides the parent compound, or that exhibits activity itself. Nonlimiting examples are the pharmaceutically acceptable salts (alternatively referred to as “physiologically acceptable salts”), and a compound, which has been alkylated or acylated at the 2′-, 3′- or 5′-position, or on the purine or pyrimidine base (a type of “pharmaceutically acceptable prodrug”). Further, the modifications can affect the biological activity of the compound, in some cases increasing the activity over the parent compound. This can easily be assessed by preparing the salt or prodrug and testing its antiviral activity according to the methods described herein, or other methods known to those skilled in the art.

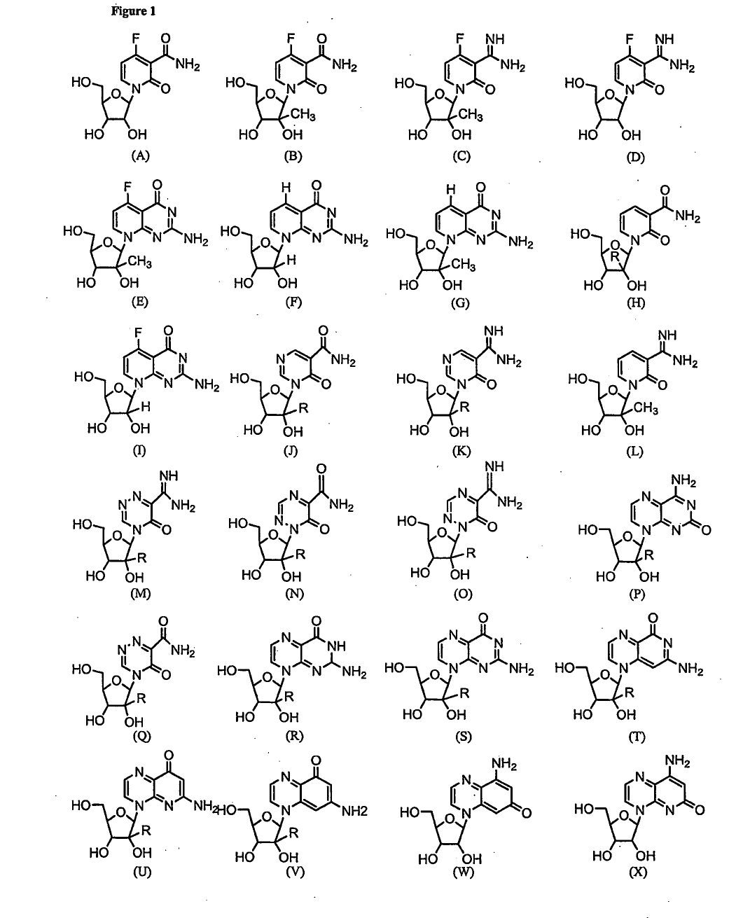

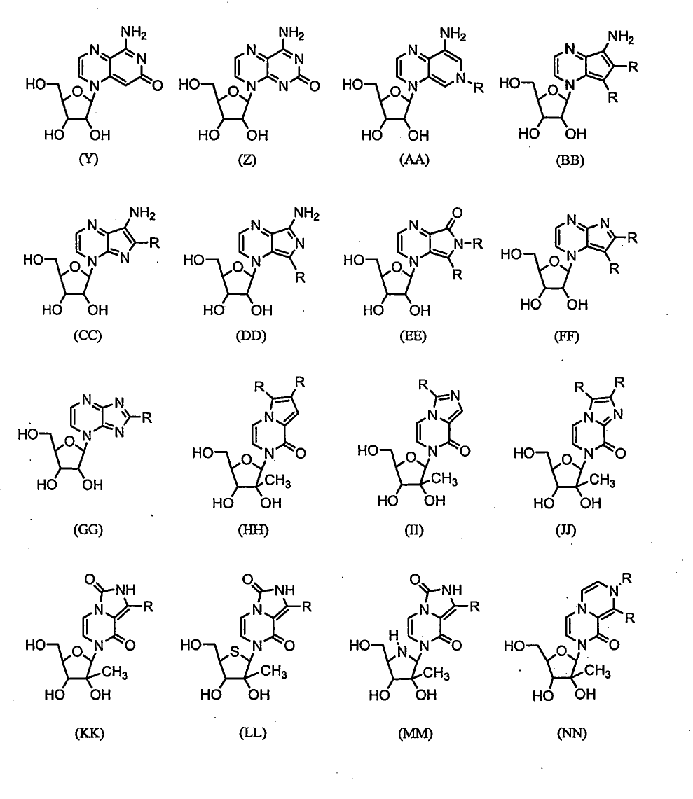

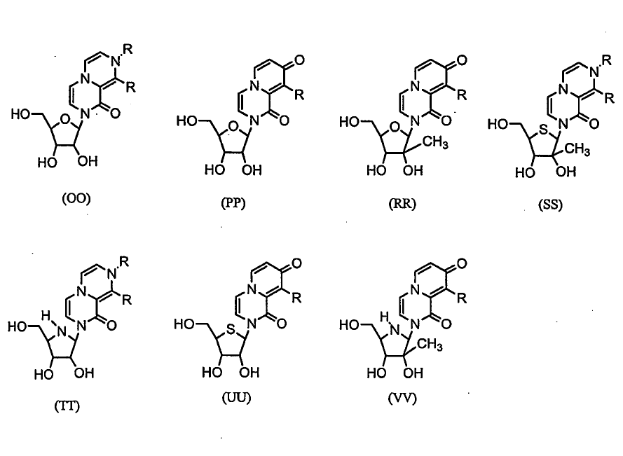

FIG. 1 depicts illustrative examples of compounds of the present invention where R is each independently H, halo, alkyl, alkenyl, alkynyl, alkoxy, alkoxyalkyl, hydroxyalkyl, cycloalkyl, nitro, cyano, OH, OR⁴, NH₂, NHR⁴, NR⁴R⁵, SH, SR⁴, CF₃, CH₂OH, CH₂F, CH₂Cl, CH₂CF₃, C(Y³)₃, C(Y³)₂C(Y³)₃, C(═O)OH, C(═O)OR⁴, C(═O)-alkyl, C(═O)-aryl, C(═O)-alkoxyalkyl, C(═O)NH₂, C(═O)NHR⁴, C(═O)N(R⁴)₂, or N₃, and R⁴, R⁵, and Y³ are as defined herein.

### II. DEFINITIONS

The term alkyl, as used herein, unless otherwise specified, refers to a saturated straight, branched, or cyclic, primary, secondary, or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Moieties with which the alkyl group can be substituted with one or more substituents selected from the group consisting of halo (F, Cl, Br or I), (e.g. CF₃, 2-Br-ethyl, CH₂F, CH₂Cl, CH₂CF₃ or CF₂CF₃), hydroxyl (e.g. CH₂OH), amino (e.g. CH₂NH₂, CH₂NHCH₃ or CH₂N(CH₃)₂), alkylamino, arylamino, alkoxy, aryloxy, nitro, azido (e.g. CH₂N₃), cyano (e.g. CH₂CN), sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term lower alkyl, as used herein, and unless otherwise specified, refers to a C₁ to C₄ saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The term alkylamino or arylamino refers to an amino group that has one or two alkyl or aryl substituents, respectively.

The term amino acid includes naturally occurring and synthetic α, β γ or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In a preferred embodiment, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of a natural or synthetic amino acid, including but not limited to α, β γ or δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “protected” as used herein and unless otherwise defined refers to a group that is added to an oxygen, nitrogen, sulfur or phosphorus atom to prevent its further reaction or for other purposes. A wide variety of oxygen and nitrogen protecting groups are known to those skilled in the art of organic synthesis (see Greene and Wuts, Protective Groups in Organic Synthesis, 3^{rd} Ed., John Wiley & Sons, Inc., New York, N.Y., 1999).

The term aryl, as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of hydroxyl, amino, alkylamino, arylamino, alkoxy; aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, 3^{rd} Ed., 1999.

The term alkaryl or alkylaryl refers to an alkyl group with an aryl substituent. The term aralkyl or arylalkyl refers to an aryl group with an alkyl substituent.

The term halo, as used herein, includes chloro, bromo, iodo, and fluoro.

The term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

As used herein, the term “substantially free of or” substantially in the absence of refers to a nucleoside composition that includes at least 85 or 90% by weight, preferably 95% to 98% by weight, and even more preferably 99% to 100% by weight, of the designated enantiomer of that nucleoside. In a preferred embodiment, in the methods and compounds of this invention, the compounds are substantially free of enantiomers.

Similarly, the term “isolated” refers to a nucleoside composition that includes at least 85 or 90% by weight, preferably 95% to 98% by weight, and even more preferably 99% to 100% by weight, of the nucleoside, the remainder comprising other chemical species or enantiomers.

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term host, as used herein, refers to a unicellular or multicellular organism in which the virus can replicate, including cell lines and animals, and preferably a human. Alternatively, the host can be carrying a part of the flavivirus, pestivirus or hepacivirus genome, whose replication or function can be altered by the compounds of the present invention. The term host specifically refers to infected cells, cells transfected with all or part of the flavivirus, pestivirus or hepacivirus genome and animals, in particular, primates (including chimpanzees) and humans. In most animal applications of the present invention, the host is a human patient. Veterinary applications, in certain indications, however, are clearly anticipated by the present invention (such as chimpanzees).

The term “pharmaceutically acceptable salt or prodrug” is used throughout the specification to describe any pharmaceutically acceptable form (such as an ester, phosphate ester, salt of an ester or a related group) of a nucleoside compound, which, upon administration to a patient, provides the nucleoside compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess antiviral activity against flavivirus, pestivirus or hepacivirus, or are metabolized to a compound that exhibits such activity.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are will known in the art. Examples of methods that can be used to obtain optical isomers of the compounds include the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce assymetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer,
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

### III. NUCLEOTIDE SALTS OR PRODRUGS

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which forth a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references: Kucera, L. S., N. Iyer, E. Leake, A. Raben, Modest E. K., D. L. W., and C. Piantadosi, “Novel membrane-interactive ether lipid analogs that inhibit infectious HIV-1 production and induce defective virus formation,” AIDS Res. Hum. Retro Viruses, 1990, 6, 491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest, “Synthesis and evaluation of novel ether lipid nucleoside conjugates for anti-HIV activity,” J. Med. Chem., 1991, 34, 1408-1414; Hosteller, K. Y., D. D. Richman, D. A. Carson, L. M. Stuhmiller, G. M. T. van Wijk, and H. van den Bosch, “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine,” Antimicrob. Agents Chemother., 1992, 36, 2025-2029; Hosetler, K. Y., L. M. Stuhmiller, H. B. Lenting, H. van den Bosch, and D. D. Richman, “Synthesis and antiretroviral activity of phospholipid analogs of azidothymidine and other antiviral nucleosides.” J. Biol. Chem., 1990, 265, 61127.

Nonlimiting examples of U.S. patents that disclose suitable lipophilic substituents that can be covalently incorporated into the nucleoside, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

### IV. ALTERNATION AND COMBINATION THERAPY

Drug-resistant variants of HCV can emerge after prolonged treatment with an antiviral agent. Drug resistance most typically occurs by mutation of a gene that encodes for an enzyme used in viral replication. The efficacy of a drug against HCV infection can be prolonged, augmented, or restored by administering the compound in combination or alternation with one or more other antiviral compounds that induce a different mutation from that caused by the principle drug. Alternatively, the pharmacokinetics, bioavailability, biodistriution or other parameter of the drug can be altered by such combination or alternation therapy. Combination therapy is typically preferred over alternation therapy because it induces multiple simultaneous stresses on the virus.

Any of the active compounds described herein can be used in combination or alternation with another antiviral compound.

Nonlimiting examples include:

### (1) Interferon

Interferons (IFNs) are glycoproteins that have been commercially available for the treatment of chronic hepatitis for nearly a decade. IFNs are produced by immune cells in response to viral infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary and a sustained response occurs in only 8%-9% of patients chronically infected with HCV (Gary L. Davis. Gastroenterology 118:S104-S114, 2000).

A number of patents disclose HCV treatments using interferon-based therapies. For example, U.S. Pat. No. 5,980,884 to Blatt et al. discloses methods for re-treatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,908,621 to Glue et al. discloses the use of polyethylene glycol modified interferon for the treatment of HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696.

(2) Ribavirin (Battaglia, A. M. et al., Ann. Pharmacother, 2000, 34, 487-494); Berenguer, M. et al. Antivir. Ther., 1998, 3 (Suppl. 3), 125-136).

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p 1304, 1989); Rebetol (Schering Plough) and Co-Pegasus (Roche). U.S. Pat. No. 3,798,209 and RE29,835 (ICN Pharmaceuticals) disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No. 4,211,771 (to ICN Pharmaceuticals) discloses the use of ribavirin as an antiviral agent. Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

(2a) Interferons and Other Anti-Viral Agents, Alone or In Combination

Schering-Plough sells ribavirin as Rebetol® capsules (200 mg) for administration to patients with HCV. The U.S. FDA has approved Rebetol capsules to treat chronic HCV infection in combination with Schering's alpha interferon-2b products Intron® A and PEG-Intron™. Rebetol capsules are not approved for monotherapy (i.e., administration independent of IntroneA or PEG-Intron), although Intron A and PEG-Intron are approved for monotherapy (i.e., administration without ribavirin). Hoffman La Roche sells ribavirin under the name Co-Pegasus in Europe and the United States, also for use in combination with interferon for the treatment of HCV. Other alpha interferon products include Roferon-A (Hoffmann-La Roche), Infergen® (Intermune, formerly Amgen's product), and Wellferon® (Wellcome Foundation) are currently FDA-approved for HCV monotherapy. Interferon products currently in development for HCV include: Roferon-A (interferon alfa-2a) by Roche, PEGASYS (pegylated interferon alfa-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

The combination of TN and ribavirin for the treatment of HCV infection has been reported to be effective in the treatment of IFN naïve patients (for example, Battaglia, A. M. et al., Ann. Pharmacother. 34:487-494, 2000). Combination treatment is effective both before hepatitis develops and when histological disease is present (for example, Berenguer, M. et al. Antivir. Ther. 3(Suppl. 3):125-136, 1998). Currently, the most effective therapy for HCV is combination therapy of pegylated interferon with ribavirin (2002 NIH Consensus Development Conference on the Management of Hepatitis C). However, the side effects of combination therapy can be significant and include hemolysis, flu-like symptoms, anemia, and fatigue (Gary L. Davis. Gastroenterology 118:S104-S114, 2000).

(3) Protease inhibitors have been developed for the treatment of Flaviviridae infections. Examples, include, but are not limited to the following:
(a) Substrate-based NS3 protease inhibitors, including alphaketoamides and hydrazinoureas
   (see, for example, Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), and inhibitors that terminate in an electrophile such as a boronic acid or phosphonate (see, for example, Llinas-Brunet et al, Hepatitis C inhibitor peptide analogues, PCT WO 99/07734);
(b) Non-substrate-based inhibitors such as 2,4,6-trihydroxy-3-nitro-benzamide derivative including RD3-4082 and RD3-4078, the former substituted on the amide with a 14 carbon chain and the latter processing apara-phenoxyphenyl group (see, for example, Sudo K. et al., Biochemical and Biophysical Research Communications, 1997, 238, 643-647; Sudo K. et al. Antiviral Chemistry and Chemotherapy, 1998, 9, 186);
(c) Phenanthrenequinones possessing activity against protease, for example in a SDS-PAGE and/or autoradiography assay, such as, for example, Sch 68631, isolated from the fermentation culture broth of Streptomyces sp., (see, for example, Chu M. et al., Tetrahedron Letters, 1996, 37, 7229-7232), and Sch 351633, isolated from the fungus Penicillium griseofulvum, which demonstrates activity in a scintillation proximity assay (see, for example, Chu M. et al., Bioorganic and Medicinal Chemistry Letters 9, 1949-1952); and
(d) Selective NS3 inhibitors, for example, based on the macromolecule elgin c, isolated from leech (see, for example, Qasim M. A. et al., Biochemistry, 1997, 36, 1598-1607). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, α-chymotrypsin, chymase and subtilisin.

Several U.S. patents disclose protease inhibitors for the treatment of HCV. Non-limiting examples include: U.S. Pat. No. 6,004,933 to Spruce et al. that discloses a class of cysteine protease inhibitors for inhibiting HCV endopeptidase; and U.S. Pat. No. 5,990,276 to Zhang et al. that discloses synthetic inhibitors of hepatitis C virus NS3 protease. The inhibitor is a subsequence of a substrate of the NS3 protease or a substrate of the NS4A cofactor. The use of restriction enzymes to treat HCV is disclosed in U.S. Pat. No. 5,538,865 to Reyes et al. Peptides useful as NS3 serine protease inhibitors of HCV are disclosed in WO 02/008251 to Corvas International, Inc., and WO 02/08187 and WO 02/008256 to Schering Corporation. HCV inhibitor tripeptides are disclosed in U.S. Pat. Nos. 6,534,523, 6,410,531, and 6,420,380 to Boehringer Ingelheim and WO 02/060926 to Bristol Myers Squibb. Diaryl peptides useful as NS3 serine protease inhibitors of HCV are disclosed in WO 02/48172 to Schering Corporation. Imidazolidinones as NS3 serine protease inhibitors of HCV are disclosed in WO 02/08198 to Schering Corporation and WO 02/48157 to Bristol Myers Squibb. WO 98/17679 to Vertex Pharmaceuticals and WO 02/48116 to Bristol Myers Squibb also disclose HCV protease inhibitors.

(4) Thiazolidine derivatives: certain of these compounds show relevant inhibition in a reverse-phase HPLC assay with an NS3/4A fusion protein and NS5A/5B substrate (see, for example, Sudo K. et al., Antiviral Research, 1996, 32, 9-18), especially compound RD-1-6250 that possesses a fused cinnamoyl moiety substituted with a long alkyl chain, (RD4 6205 and RD4 6193);
(5) Thiazolidines and benzanilides: for example, see Kakiuchi N. et al. J. EBS Letters 421, 217-220, and Takeshita N. et al. Analytical Biochemistry, 1997, 247, 242-246;
(6) Helicase inhibitors: see, for example, Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554;
(7) Polymerase inhibitors:
(a) nucleotide analogues like gliotoxin (see, for example, Ferrari R. et al. Journal of Virology, 1999, 73, 1649-1654);
(b) the natural product cerulenin (see, for example, Lohmann V. et al., Virology, 1998, 249, 108-118); and
(c) non-nucleoside polymerase inhibitors, including, for example, compound R803 (see, for example, WO 04/018463 A2 and WO 03/040112 A1, both to Rigel Pharmaceuticals, Inc.); substituted diamine pyrimidines (see, for example, WO 03/063794 A2 to Rigel Pharmaceuticals, Inc.); benzimidazole derivatives (see, for example, Bioorg. Med. Chem. Lett., 2004, 14:119-124 and Bioorg. Med. Chem. Lett., 2004, 14:967-971, both to Boehringer Ingelheim Corporation); N,N-disubstituted phenylalanines (see, for example, J. Biol. Chem., 2003, 278:9495-98 and J. Med. Chem., 2003, 13:1283-85, both to Shire Biochem, Inc.); substituted thiophene-2-carboxylic acids (see, for example, Bioorg. Med. Chem. Lett., 2004, 14:793-796 and Bioorg. Med. Chem. Lett., 2004, 14:797-800, both to Shire Biochem, Inc.); α,γ-diketoacids (see, for example, J. Med. Chem., 2004, 14-17 and WO 00/006529 A1, both to Merck & Co., Inc.); and meconic acid derivatives (see, for example, Bioorg. Med. Chem. Lett., 2004, 3257-3261, WO 02/006246 A1 and WO03/062211 A1, all to IRBM Merck & Co., Inc.);
   (8) Antisense phosphorothioate oligodeoxynucleotides (S-ODN): complementary, for example, to sequence stretches in the 5′ non-coding region (NCR) of the HCV virus (see, for example, Alt M. et al., Hepatology, 1995, 22, 707-717), or to nucleotides 326-348 comprising the 3′ end of the NCR and nucleotides 371-388 located in the core coding region of the HCV RNA (see, for example, Alt M. et al., Archives of Virology, 1997, 142, 589-599; Galderisi U. et al., Journal of Cellular Physiology, 1999, 181, 251-257);
   (9) Inhibitors of MES-dependent translation: (see, for example, Ikeda N et al., Agent for the prevention and treatment of hepatitis C, Japanese Patent Pub. R-08268890; Kai Y. et al. Prevention and treatment of viral diseases, Japanese Patent Pub. JP-10101591).
   (10) Nuclease-resistant ribozymes: see, for example, Maccjak, D. J. et al., Hepatology 1999, 30, abstract 995; U.S. Pat. No. 6,043,077 to Barber et al.; and U.S. Pat. Nos. 5,869,253 and 5,610,054 to Draper et al.
   (11) Nucleoside analogs have also been developed for the treatment of Flaviviridae infections.

Idenix Pharmaceuticals discloses branched nucleosides, and their use in the treatment of HCV and flaviviruses and pestiviruses in US Patent Publication Nos. 2003/0050229 A1, 2004/0097461 A1, 2004/0101535 A1, 2003/0060400 A1, 2004/0102414 A1, 2004/0097462 A1, and 2004/0063622 A1 which correspond to International Publication Nos. WO 01/90121 and WO 01/92282. A method for the treatment of flavivirus and pestivirus infections, including hepatitis C infection, in humans and other host animals is disclosed in the Idenix publications that include administering an effective amount of a biologically active 1′, 2′, 3′ or 4′-branched β-D or β-L nucleoside or a pharmaceutically acceptable salt or prodrug thereof, either alone or in combination with one or more other anti-viral agents, and optionally in a pharmaceutically acceptable carrier. See also U.S. Patent Publication Nos. 2004/0006002 and 2004/0006007 as well as WO 03/026589 and WO 03/026675. Idenix Pharmaceuticals also discloses in US Patent Publication No. 2004/0077587 pharmaceutically acceptable branched nucleoside prodrugs, and their use in the treatment of HCV and flaviviruses and pestiviruses in prodrugs. See also PCT Publication Nos. WO 04/002422, WO 04/002999, and WO 04/003000. Further, Idenix Pharmaceuticals also discloses in WO 04/046331 Flaviviridae mutations caused by biologically active 2′-branched β-D or β-L nucleosides or a pharmaceutically acceptable salt or prodrug thereof.

Biota Inc. discloses various phosphate derivatives of nucleosides, including 1′, 2′, 3′ or 4′-branched β-D or β-L nucleosides, for the treatment of hepatitis C infection, in International Patent Publication WO 03/072757.

Emory University and the University of Georgia Research Foundation, Inc. (UGARF) discloses the use of 2′-fluoronucleosides for the treatment of HCV in U.S. Pat. No. 6,348,587. See also US Patent Publication No. 2002/0198171 and International Patent Publication WO 99/43691.

BioChem Pharma Inc. (now Shire Biochem, Inc.) discloses the use of various 1,3-dioxolane nucleosides for the treatment of a Flaviviridae infection in U.S. Pat. No. 6,566,365. (See also U.S. Pat. Nos. 6,340,690 and 6,605,614; US Patent Publication Nos. 2002/0099072 and 2003/0225037; and International Publication No. WO 01/32153 and WO 00/50424.) BioChem Pharma Inc. also discloses various other 2′-halo, 2′-hydroxy and 2′-alkoxy nucleosides for the treatment of a Flaviviridae infection in US Patent Publication No. 2002/0019363 as well as International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. (See also WO 98/16184, WO 01/68663, and WO 02/03997.)

U.S. Pat. No. 6,660,721, US Patent Publication Nos. 2003/083307 A1, 2003/008841 A1, and 2004/0110718, and International Patent Publication Nos. WO 02/18404, WO 02/100415, WO 02/094289, and WO 04/043159, all filed by F. Hoffmann-La Roche AG, disclose various nucleoside analogs for the treatment of HCV RNA replication.

Pharmasset Limited discloses various nucleosides and antimetabolites for the treatment of a variety of viruses, including Flaviviridae, and in particular HCV, in US Patent Publication Nos. 2003/0087873, 2004/0067877, 2004/0082574, 2004/0067877, 2004/002479, 2003/0225029, and 2002/00555483, as well as International Patent Publication Nos. WO 02/32920, WO 01/79246, WO 02/48165, WO 03/068162, WO 03/068164 and WO 2004/013298.

Merck & Co., Inc. and Isis Pharmaceuticals disclose various nucleosides, particularly several pyrrolopyrimidine nucleosides, for the treatment of viruses that replicate through an RNA-dependent RNA polymerase mechanism, including Flaviviridae and HCV in particular (see US Patent Publication Nos. 2002/0147160, 2004/0072788, 2004/0067901, and 2004/0110717, and corresponding International Patent Publication Nos. WO 02/057425 (PCT/US02/01531; filed Jan. 18, 2002) and WO 02/057287 (PCT/US02/03086; filed Jan. 18, 2002; see also WO 2004/000858, WO 2004/003138, WO 2004/007512, and WO 2004/009020).

US Patent Publication No. 2003/028013 A1 and International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

US Patent Publication No. 2004/0063658 and International Patent Publication Nos. WO 03/093290 and WO 04/028481 to Genelabs Technologies disclose various base modified derivatives of nucleosides, including 1′, 2′, 3′ or 4′-branched β-D or β-L nucleosides, for the treatment, of hepatitis C infection.

Eldrup et al. (Oral Session V, Hepatitis C Virus, Flaviviridae; 16^{th} International Conference on Antiviral Research (Apr. 27, 2003, Savannah, Ga.) p. A75) and Olsen et al. (Id. at p. A76) described the structure activity relationship of 2′-modified nucleosides for inhibition of HCV.

Bhat et al (Oral Session V, Hepatitis C Virus, Flaviviridae; 16^{th} International Conference on Antiviral Research (Apr. 27, 2003, Savannah, Ga.); p A75) describe the synthesis and pharmacokinetic properties of nucleoside analogues as possible inhibitors of HCV RNA replication. The authors report that 2′-modified nucleosides demonstrate potent inhibitory activity in cell-based replicon assays.

(12) Other miscellaneous compounds developed for the treatment of Flaviviridae infections include 1-amino-alkylcyclohexanes (for example, U.S. Pat. No. 6,034,134 to Gold et al.), alkyl lipids (for example, U.S. Pat. No. 5,922,757 to Chojkier et al.), vitamin E and other antioxidants (for example, U.S. Pat. No. 5,922,757 to Chojkier et al.), squalene, amantadine, bile acids (for example, U.S. Pat. No. 5,846,964 to Ozeki et al.), N-(phosphonoacetyl)-L-aspartic acid (for example, U.S. Pat. No. 5,830,905 to Diana et al.), benzenedicarboxamides (for example, U.S. Pat. No. 5,633,388 to Diana et al.), polyadenylic acid derivatives (for example, U.S. Pat. No. 5,496,546 to Wang et al.), 2′,3′-dideoxyinosine (for example, U.S. Pat. No. 5,026,687 to Yarchoan et al.), benzimidazoles (for example, U.S. Pat. No. 5,891,874 to Colacino et al.), plant extracts (for example, U.S. Pat. No. 5,837,257 to Tsai et al., U.S. Pat. No. 5,725,859 to Omer et al., and U.S. Pat. No. 6,056,961), and piperidenes (for example, U.S. Pat. No. 5,830,905 to Diana et al.).

Still other compounds include, for example: Interleukin-10 by Schering-Plough, IP-501 by Interneuron, Merimebodib VX-497 by Vertex, AMANTADINE® (Symmetrel) by Endo Labs Solvay, HEPTAZYME® by RPI, IDN-6556 by Idun Pharma., XTL-002 by XTL., HCV/MF59 by Chiron, CIVACIR® (Hepatitis C Immune Globulin) by NABI, LEVOVIRIN® by ICN/Ribapharm, VIRAMIDINE® by ICN/Ribapharm, ZADAXIN® (thymosin alfa-1) by Sci Clone, thymosin plus pegylated interferon by Sci Clone, CEPLENE® (histamine dihydrochloride) by Maxim, VX 950/LY 570310 by Vertex/Eli Lilly, ISIS14803 by Isis Pharmaceutical/Elan, IDN-6556 by Idun Pharmaceuticals, Inc., JTK 003 by AKROS Pharma, BILN-2061 by Boehringer Ingelheim, CellCept (mycophenolate mofetil) by Roche, T67, a β-tubulin inhibitor, by Tularik, a therapeutic vaccine directed to E2 by Innogenetics, FK788 by Fujisawa Healthcare, Inc., IdB 1016 (Sifiphos, oral silybin-phosphatdylcholine phytosome), RNA replication inhibitors (VP50406) by ViroPharma/Wyeth, therapeutic vaccine by Intercell, therapeutic vaccine by Epimmune/Genencor, IBES inhibitor by Anadys, ANA 245 and ANA 246 by Anadys, immunotherapy (Therapore) by Avant, protease inhibitor by Corvas/SChering, helicase inhibitor by Vertex, fusion inhibitor by Trimeris, T cell therapy by CellExSys, polymerase inhibitor by Biocryst, targeted RNA chemistry by PTC Therapeutics, Dication by Immtech, Int., protease inhibitor by Agouron, protease inhibitor by Chiron/Medivir, antisense therapy by AVI BioPharma, antisense therapy by Hybridon, hemopurifier by Aethlon Medical, therapeutic vaccine by Merix, protease inhibitor by Bristol-Myers Squibb/Axys, Chron-VacC, a therapeutic vaccine, by Tripep, UT 231B by United Therapeutics, protease, helicase and polymerase inhibitors by Genelabs Technologies, IRES inhibitors by Immusol, R803 by Rigel Pharmaceuticals, INFERGEN® (interferon alphacon-1) by InterMune, OMNIFERON® (natural interferon) by Viragen, ALBUFERON® by Human Genome Sciences, REBIF® (interferon beta-1a) by Ares-Serono, Omega Interferon by BioMedicine, Oral Interferon Alpha by Amarillo Biosciences, interferon gamma, interferon tau, and Interferon gamma-1b by InterMune.

### V. PHARMACEUTICAL COMPOSITIONS

A host, including a human, infected with flavivirus, pestivirus or hepacivirus can be treated by administering to that host an effective amount of an active compound of the present invention, or a pharmaceutically acceptable prodrug or salt thereof, optionally in the presence of a pharmaceutically acceptable carrier or diluent. The active materials can be administered by any appropriate route, for example, orally, parenterally, topically, intravenously, intradermally, or subcutaneously, in liquid or solid form.

Nonlimiting examples of doses of the compound infection will be in the range from 1 to 80 mg/kg, 1 to 70 mg/kg, 1 to 60 mg/kg, 1 to 50 mg/kg, or 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent nucleoside to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3000 mg, preferably 70 to 1400 mg of active ingredient per unit dosage form. A oral dosage of 50-1000 mg is usually convenient.

Ideally the active ingredient should be administered to achieve peak plasma concentrations of the active compound of from about 0.2 to 70 preferably about 1.0 to 10 μM. This may be achieved, for example, by the intravenous injection of a 0.1 to 5% solution of the active ingredient, optionally in saline, or administered as bolus of the active ingredient.

The concentration of active compound in the drug composition will depend on absorption, bioavailability, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories, or other antivirals, including other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives then is introduced into the container. The container is swirled by hand to free lipid material from its sides and to disperse lipid aggregates, thereby forming the liposomal suspension.

### VI. PROCESSES FOR THE PREPARATION OF ACTIVE COMPOUNDS

The nucleosides of the present invention can be synthesized by any means known in the art. In particular, the synthesis of the present nucleosides can be achieved by either alkylating the appropriately modified sugar, followed by glycosylation or glycosylation followed by alkylation of the nucleoside. The following non-limiting embodiments illustrate some general methodology to obtain the nucleosides of the present invention.

### General Synthesis of 1′-C-Branched Nucleosides

1′-C-Branched ribonucleosides of the following structure:

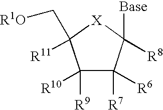

wherein Base, R¹, R⁶, R⁷, R⁸, R⁹, R¹⁰, R¹¹ and X are as defined herein can be prepared by one of the following general methods.
1) Modification from the Lactone

The key starting material for this process is an appropriately substituted lactone. The lactone can be purchased or can be prepared by any known means including standard epimerization, substitution and cyclization techniques. The lactone can be optionally protected with a suitable protecting group, preferably with an acyl or silyl group, by methods well known to those skilled in the art, as taught by Greene et al. Protective Groups in Organic Synthesis, John Wiley and Sons, Second Edition, 1991. The protected lactone can then be coupled with a suitable coupling agent, such as an organometallic carbon nucleophile, such as a Grignard reagent, an organolithium, lithium dialkylcopper or R⁶—SiMe₃ in TBAF with the appropriate non-protic solvent at a suitable temperature, to give the 1′-alkylated sugar.

The optionally activated sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a Lewis acid, such as tin tetrachloride, titanium tetrachloride or trimethylsilyltriflate in the appropriate solvent at a suitable temperature.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 1′-C-branched ribonucleoside is desired. The synthesis of a ribonucleoside is shown in Scheme 1. Alternatively, deoxyribo-nucleoside is desired. To obtain these nucleosides, the formed ribonucleoside can optionally be protected by methods well known to those skilled in the art, as taught by Greene et al., Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

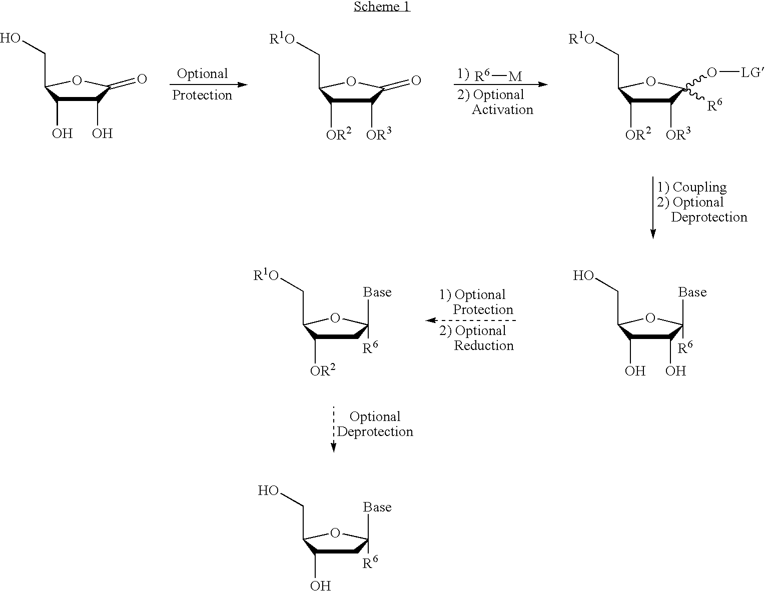

### 2) Alternative Method for the Preparation of 1′-C-Branched Nucleosides

The key starting material for this process is an appropriately substituted hexose. The hexose can be purchased or can be prepared by any known means including standard epimerization (e.g. via alkaline treatment), substitution and coupling techniques. The hexose can be selectively protected to give the appropriate hexa-furanose, as taught by Townsend Chemistry of Nucleosides and Nucleotides, Plenum Press, 1994.

The 1′-hydroxyl can be optionally activated to a suitable leaving group such as an acyl group or a halogen via acylation or halogenation, respectively. The optionally activated sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a Lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

The 1′-CH₂—OH, if protected, can be selectively deprotected by methods well known in the art. The resultant primary hydroxyl can be functionalized to yield various C-branched nucleosides. For example, the primary hydroxyl can be reduced to give the methyl, using a suitable reducing agent. Alternatively, the hydroxyl can be activated prior to reduction to facilitate the reaction; i.e. via the Barton reduction. In an alternate embodiment, the primary hydroxyl can be oxidized to the aldehyde, then coupled with a carbon nucleophile, such as a Grignard reagent, an organolithium, lithium dialkylcopper or R⁶—SiMe₃ in TBAF with the appropriate non-protic solvent at a suitable temperature.

In a particular embodiment, the 1′-C-branched ribonucleoside is desired. The synthesis of a ribonucleoside is shown in Scheme 2. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

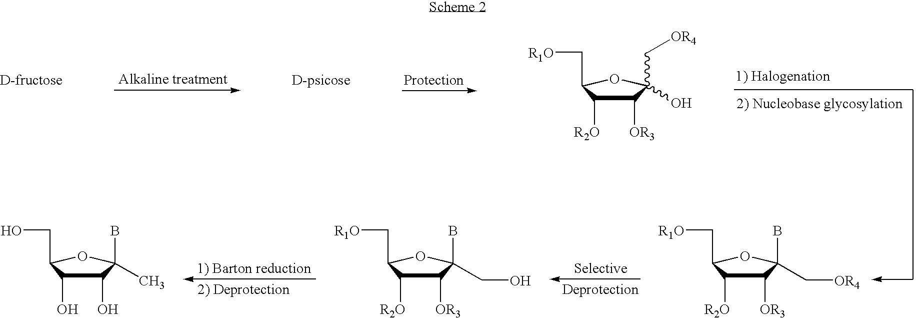

In addition, the L-enantiomers corresponding to the compounds of the invention can be prepared following the same general methods (1 or 2), beginning with the corresponding L-sugar or nucleoside L-enantiomer as starting material.

### General Synthesis of 2′-C-Branched Nucleosides

2′-C-Branched ribonucleosides of the following structure:

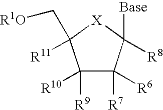

wherein Base, R¹, R⁶, R⁷, R⁸, R⁹, R¹⁰, R¹¹ and X are as defined herein can be prepared by one of the following general methods.
1) Glycosylation of the Nucleobase with an Appropriately Modified Sugar

The key starting material for this process is an appropriately substituted sugar with a 2′-OH and 2′-H, with the appropriate leaving group (LG), for example an acyl group or a halogen. The sugar can be purchased or can be prepared by any known means including standard epimerization, substitution, oxidation and reduction techniques. The substituted sugar can then be oxidized with the appropriate oxidizing agent in a compatible solvent at a suitable temperature to yield the 2′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN; NaOCl in HOAc, copper chromite, copper oxide, Raney nickel, palladium acetate, Meerwin-Pondorf-Verley reagent (aluminum t-butoxide with another ketone) and N-bromosuccinimide.

Then coupling of an organometallic carbon nucleophile, such as a Grignard reagent, an organolithium, lithium dialkylcopper or R⁶—SiMe₃ in TBAF with the ketone with the appropriate non-protic solvent at a suitable temperature, yields the 2′-alkylated sugar. The alkylated sugar can be optionally protected with a suitable protecting group, preferably with an acyl or silyl group, by methods well known to those skilled in the art, as taught by Greene et al. Protective Groups in Organic Synthesis, John Wiley and Sons, Second Edition, 1991.

The optionally protected sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a Lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 2′-C-branched ribonucleoside is desired. The synthesis of a ribonucleoside is shown in Scheme 3. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

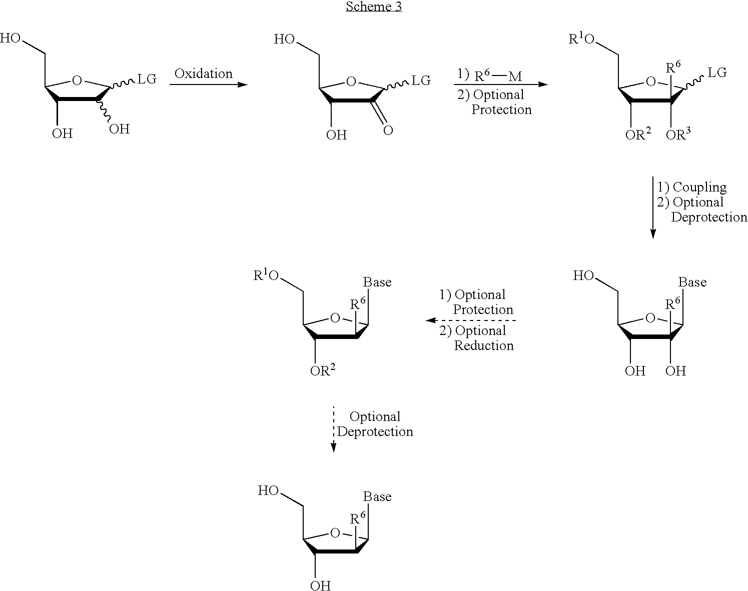

### 2) Modification of a Pre-Formed Nucleoside

The key starting material for this process is an appropriately substituted nucleoside with a 2′-OH and 2′-H. The nucleoside can be purchased or can be prepared by any known means including standard coupling techniques. The nucleoside can be optionally protected with suitable protecting groups, preferably with acyl or silyl groups, by methods well known to those skilled in the art, as taught by Greene et al. Protective Groups in Organic Synthesis, John Wiley and Sons, Second Edition, 1991.

The appropriately protected nucleoside can then be oxidized with the appropriate oxidizing agent in a compatible solvent at a suitable temperature to yield the 2′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Subsequently, the nucleoside can be deprotected by methods well known to those skilled in the art, as taught by GreeneGreene et al., Protective Groups in Organic Synthesis, John Wiley and Sons, Second Edition, 1991.

In a particular embodiment, the 2′-C-branched ribonucleoside is desired. The synthesis of a ribonucleoside is shown in Scheme 4. Alternatively, deoxyribo-nucleoside is desired. To obtain these nucleosides, the formed ribonucleoside can optionally be protected by methods well known to those skilled in the art, as taught by Greene et al., Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

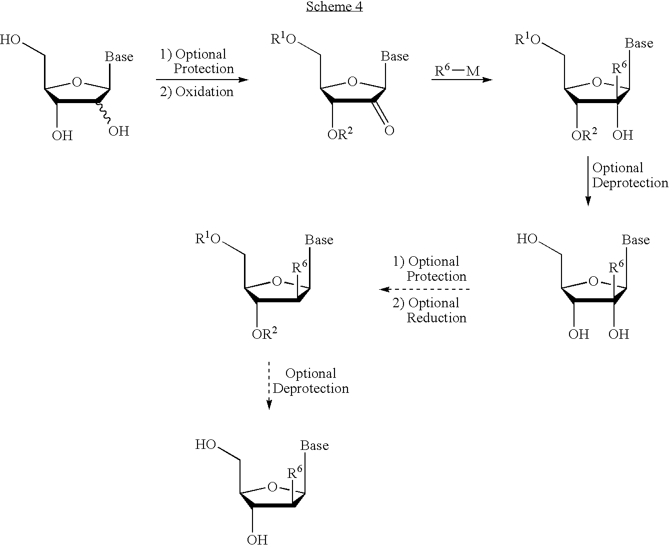

In another embodiment of the invention, the L-enantiomers are desired. Therefore, the L-enantiomers can be corresponding to the compounds of the invention can be prepared following the same foregoing general methods, beginning with the corresponding L-sugar or nucleoside L-enantiomer as starting material.

### General Synthesis of 3′-C-Branched Nucleosides

3′-C-Branched ribonucleosides of the following structure:

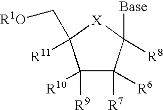

wherein Base, R¹, R⁶, R⁷, R⁸, R⁹, R¹⁰, R¹¹ and X are as defined herein can be prepared by one of the following general methods.
1) Glycosylation of the Nucleobase with an Appropriately Modified Sugar

The key starting material for this process is an appropriately substituted sugar with a 3′-OH and 3′-H, with the appropriate leaving group (LG), for example an acyl group or a halogen. The sugar can be purchased or can be prepared by any known means including standard epimerization, substitution, oxidation and reduction techniques. The substituted sugar can then be oxidized with the appropriate oxidizing agent in a compatible solvent at a suitable temperature to yield the 3′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Then coupling of an organometallic carbon nucleophile, such as a Grignard reagent, an organolithium, lithium dialkylcopper or R⁶—SiMe₃ in TBAF with the ketone with the appropriate non-protic solvent at a suitable temperature, yields the 3′-C-branched sugar. The 3′-C-branched sugar can be optionally protected with a suitable protecting group, preferably with an acyl or silyl group, by methods well known to those skilled in the art, as taught by Greene et al. Protective Groups in Organic Synthesis, John Wiley and Sons, Second Edition, 1991.

The optionally protected sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 3′-C-branched ribonucleoside is desired. The synthesis of a ribonucleoside is shown in Scheme 5. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

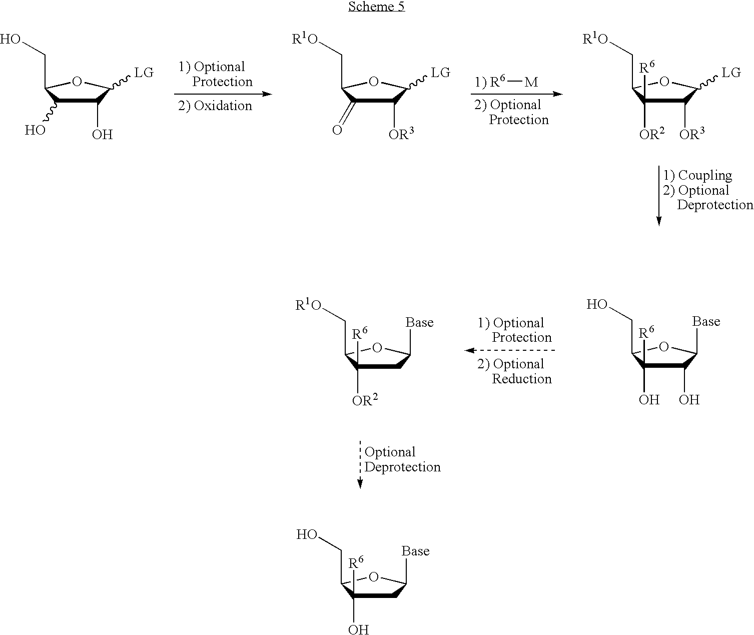

### 2) Modification of a Pre-Formed Nucleoside

The key starting material for this process is an appropriately substituted nucleoside with a 3′-OH and 3′-H. The nucleoside can be purchased or can be prepared by any known means including standard coupling techniques. The nucleoside can be optionally protected with suitable protecting groups, preferably with acyl or silyl groups, by methods well known to those skilled in the art, as taught by Greene et al. Protective Groups in Organic Synthesis, John Wiley and Sons, Second Edition, 1991.

The appropriately protected nucleoside can then be oxidized with the appropriate oxidizing agent in a compatible solvent at a suitable temperature to yield the 2′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrOᵣ CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 3′-C-branched ribonucleoside is desired. The synthesis of a ribonucleoside is shown in Scheme 6. Alternatively, deoxyribo-nucleoside is desired. To obtain these nucleosides, the formed ribonucleoside can optionally be protected by methods well known to those skilled in the art, as taught by Greene et al., Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

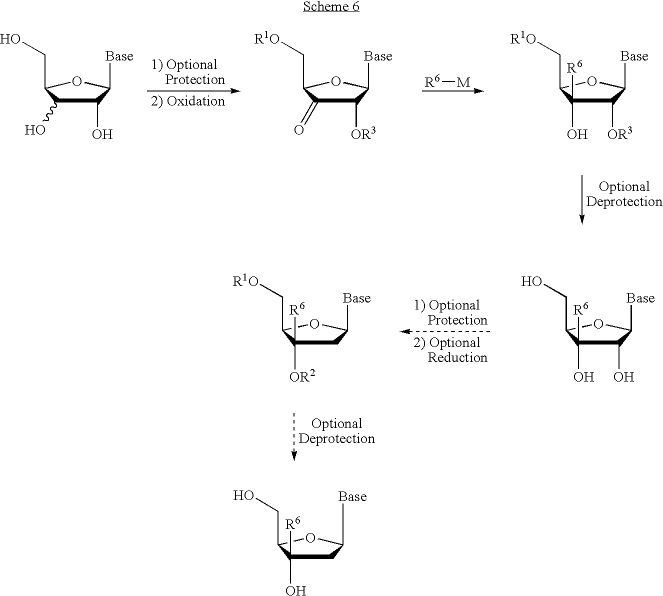

In another embodiment of the invention, the L-enantiomers are desired. Therefore, the L-enantiomers can be corresponding to the compounds of the invention can be prepared following the same foregoing general methods, beginning with the corresponding L-sugar or nucleoside L-enantiomer as starting material.

### General Synthesis of 4′-C-Branched Nucleosides

4′-C-Branched ribonucleosides of the following structure:

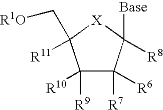

wherein Base, R¹, R⁶, R⁷, R⁸, R⁹, R¹⁰, R¹¹ and X are as defined herein can be prepared by one of the following general methods.
1) Modification from the Pentodialdo-Furanose

The key starting material for this process is an appropriately substituted pentodialdo-furanose. The pentodialdo-furanose can be purchased or can be prepared by any known means including standard epimerization, substitution and cyclization techniques.

In a preferred embodiment, the pentodialdo-furanose is prepared from the appropriately substituted hexose. The hexose can be purchased or can be prepared by any known means including standard epimerization (e.g. via alkaline treatment), substitution and coupling techniques. The hexose can be either in the furanose form, or cyclized via any means known in the art, such as methodology taught by Townsend Chemistry of Nucleosides and Nucleotides, Plenum Press, 1994, preferably by selectively protecting the hexose, to give the appropriate hexafuranose.

The 4′-hydroxymethylene of the hexafuranose then can be oxidized with the appropriate oxidizing agent in a compatible solvent at a suitable temperature to yield the 4′-aldo-modified sugar. Possible oxidizing agents are Swern reagents, Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide, though preferably using H₃PO₄, DMSO and DCC in a mixture of benzene/pyridine at room temperature.

Then, the pentodialdo-furanose can be optionally protected with a suitable protecting group, preferably with an acyl or silyl group, by methods well known to those skilled in the art, as taught by Greene et al. Protective Groups in Organic Synthesis, John Wiley and Sons, Second Edition, 1991. In the presence of a base, such as sodium hydroxide, the protected pentodialdo-furanose can then be coupled with a suitable electrophilic alkyl, halogeno-alkyl (i.e. CF₃), alkenyl or alkynyl (i.e. allyl), to obtain the 4′-alkylated sugar. Alternatively, the protected pentodialdo-furanose can be coupled with the corresponding carbonyl, such as formaldehyde, in the presence of a base, such as sodium hydroxide, with the appropriate polar solvent, such as dioxane, at a suitable temperature, which can then be reduced with an appropriate reducing agent to give the 4′-alkylated sugar. In one embodiment, the reduction is carried out using PhOC(S)Cl, DMAP, preferably in acetonitrile at room temperature, followed by treatment of ACCN and TMSS refluxed in toluene.

The optionally activated sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 4′-C-branched ribonucleoside is desired. Alternatively, deoxyribonucleoside is desired. To obtain these deoxyribo-nucleosides, a formed ribo-nucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

In another embodiment of the invention, the L-enantiomers are desired. Therefore, the L-enantiomers can be corresponding to the compounds of the invention can be prepared following the same foregoing general methods, beginning with the corresponding L-pentodialdo-furanose as starting material.

The present invention is described by way of illustration, in the following examples. It will be understood by one of ordinary skill in the art that these examples are in no way limiting and that variations of detail can be made without departing from the spirit and scope of the present invention.

### General Synthesis of Pyrazinone Carboxamide Nucleoside Analogs

1) Preparation of 2-hydroxy-3-carboxamidopyrazine

The key starting material in this synthesis is diethylaminomalonate, which is commercially available or can be synthesized by any means known by those skilled in the art. Sodium hydrogencarbonate (sodium bicarbonate) is added to aqueous diethylaminomalonate hydrochloride and, after extraction, the organic phase is evaporated and treated with ammonia/methanol to provide aminomalondiamide quantitatively. Alternatively, diethylaminomalonate is reacted with sodium nitrate in acetyl alcohol and ammonium hydroxide, then with ammonia in the presence of H₂/Pd catalyst to provide aminomalondiamide. Aminomalondiamide next is solubilized in water, and glyoxal sodium bisulfite hemihydrate is added for coupling and cyclization reactions. Hydrogen peroxide is then added to hydroxylate the aromatic ring and to yield the desired carboxamidopyrazine as a precipitate. Dialkyl and diacyl peroxides as well as Fenton's reagent (hydrogen peroxide and ferrous sulfate mixture) may be used in place of hydrogen peroxide, but yields are somewhat lower than with hydrogen peroxide and unwanted side products may result. Scheme 7 shows these reaction sequences:

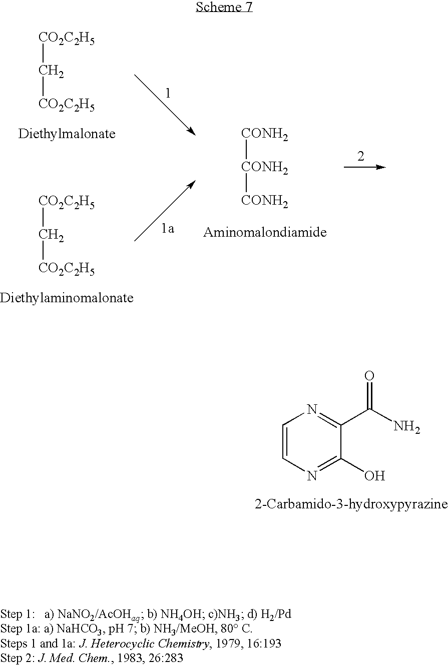

Alternatively, 3-hydroxypyrazinoic acid may be utilized as a starting material, which is reacted methanol in the presence of sulfuric acid to provide the methyl ester derivative. The methyl ester derivative then is reacted with ammonium hydroxide to provide the desired 3-hydroxy-2-carboxamidopyrazine product, as shown in Scheme 7a.

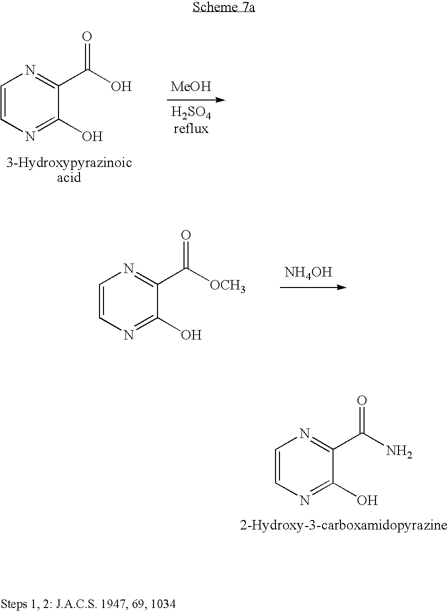

2) Condensation Reaction with Protected Ribofuranosyl

The 2-carbamido-3-hydroxypyrazine (3-hydroxy-2-pyrazinecarboxamide) product obtained from Scheme 7 is next reacted with a ribofuranosyl ring whose hydroxy groups have been protected by methods well known to those skilled in the art, such as by reaction with benzoyl or acyl groups, as taught by Greene et al. Protective Groups in Organic Synthesis, John Wiley and Sons, Second Edition, 1991. In a preferred method, the 3-hydroxy-2-pyrazinecarboxamide is silylated, reacted with the appropriately protected ribofuranosyl ring of choice, then deprotected by methods well known to those skilled in the art such as those taught by Greene et al. (Id.), and purified by reverse phase column chromatography to provide both α- and β-anomers of the 3-carboxamidopyrazin-2-one product, as shown in Scheme 8.

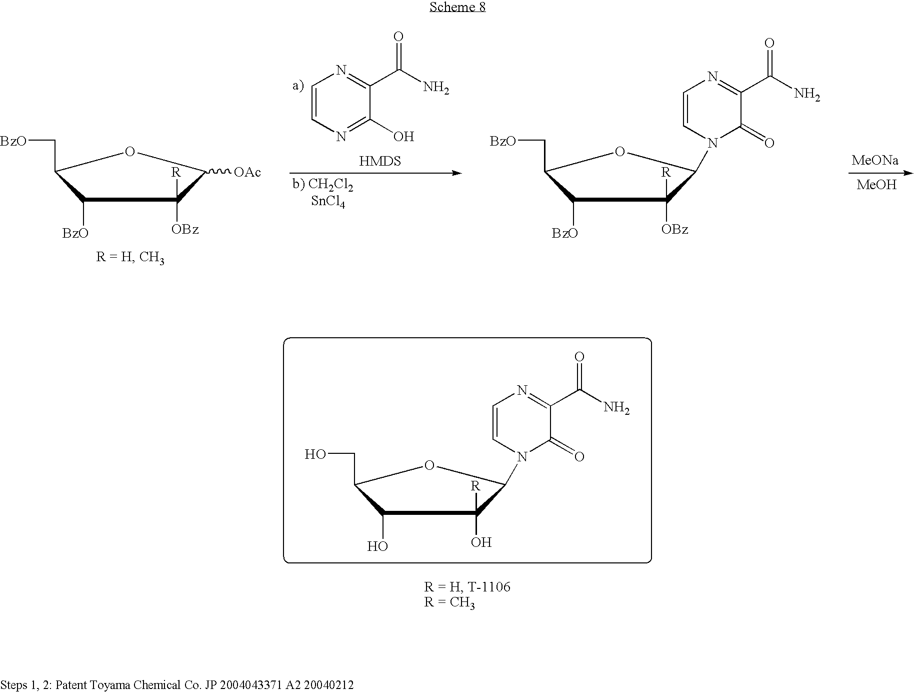

Alternatively, the reagents used in Step 2 of the process given in Scheme 8 above can be replaced with ammonia and methanol to provide the identical product, as shown in Scheme 8a below.

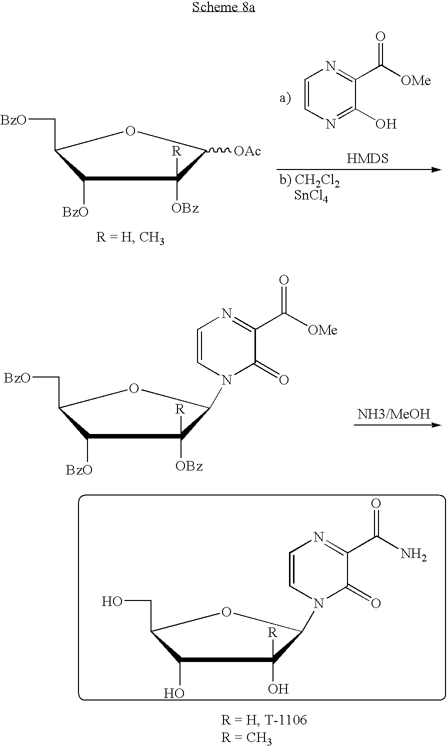

### General Preparation of Amidinopyrazinone Nucleoside Analogs

Amidinopyrazinone nucleoside analogs are synthesized using a 2-carboxamido-pyrazin-3-one nucleoside as shown in Scheme 8 as a starting material. The 2-carboxamido-pyrazin-3-one nucleoside is reacted with Lawesson's reagent or P₂S₅ to provide a 2-thioaminopyrazin-3-one nucleoside intermediate, which is then reacted with methanol and ammonia to deprotect the sugar ring and to give the desired 2-amidino-pyrazin-3-one nucleoside product.

Alternatively, a 2-thioaminopyrazin-3-one intermediate can be prepared using 2-carboxamido-pyrazin-3-one as a starting material. The 2-thioaminopyrazin-3-one then can be condensed with a protected ribofuranosyl ring (as shown in Scheme 8 above), and the resulting nucleoside analog treated with ammoniated methanol to provide 2-amidino-pyrazin-3-one nucleoside analog as the desired product.

In a second alternative process, a 2-cyano-pyrazin-3-one β-D or β-L nucleoside intermediate that is appropriately protected at its 2′-, 3′- and 5′-positions such as taught by Greene et al., Protective Groups in Organic Synthesis, John Wiley and Sons, Second Edition, 1991, and known to those skilled in the art, may be prepared by reacting an appropriately protected 2-carboxamido-pyrazin-3-one β-D or β-L nucleoside with pyridine and (CF₃CO₂)₂O in THF to provide the cyano intermediate, which then is reacted with NH₄Cl and NH₃ at approximately 85° C. to provide the desired amidinopyrazinone final product.

Scheme 9 depicts the steps in each of these alternative processes.

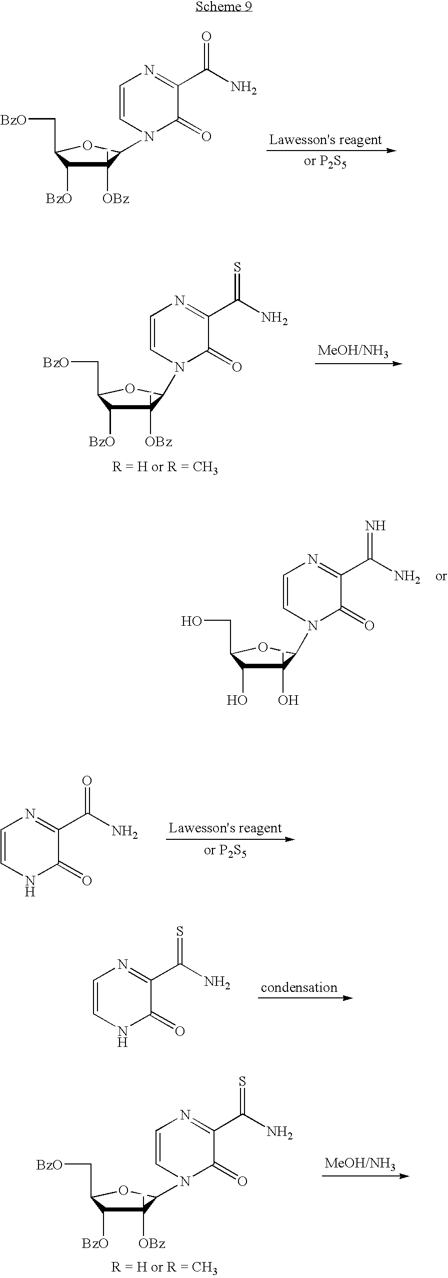

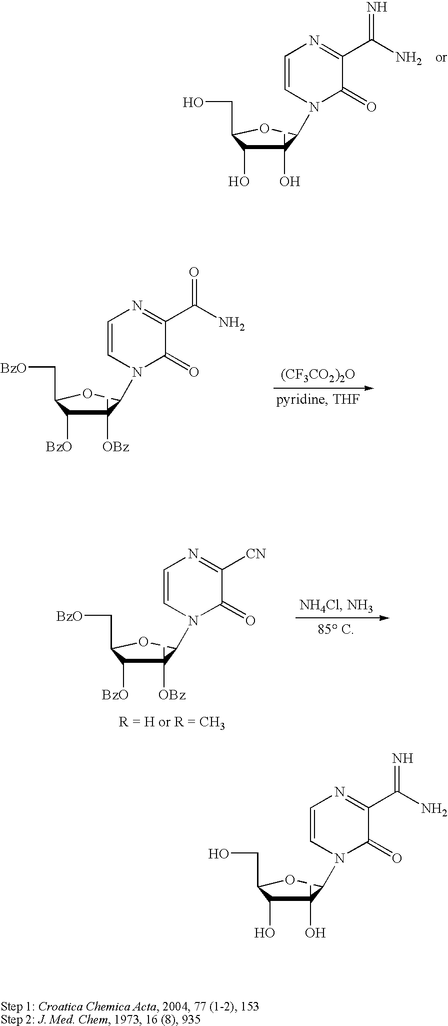

General Synthesis of Pyrazinone Carboxamide Methyl Ester Nucleoside Analogs

Synthesis of pyrazinone carboxamide methyl ester nucleoside analogs begins with a 2-carboxylic acid derivative of pyrazin-3-one that is reacted with SOCl₂ in methanol to produce the 2-methyl ester. The

2-methyl ester then is condensed with a protected ribofuranosyl ring as provided in Schemes 8 and 8a above, to give the desired 2-methyl-ester pyrazin-3-one nucleoside product. These steps are shown in

Scheme 10.

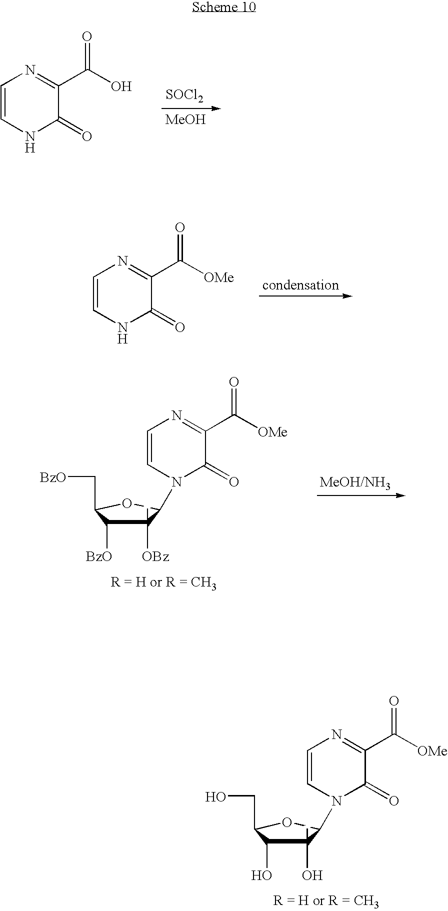

General Synthesis of Pyridinone Carboxylic Acid and Carboxamide Nucleoside Analogs

1) Condensation Reaction

A ribofuranosyl ring having appropriately protected hydroxy groups is utilized as a starting material.

Protection of the hydroxy groups is generally by reaction with acyl, benzoyl or other appropriate protective groups as taught by Greene et al. Protective Groups in Organic Synthesis, John Wiley and Sons, Second Edition, 1991, and known to those skilled in the art. The protected ribofuranosyl ring is condensed with 2-hydroxynicotinic acid in the presence of BSA (O,N-bistrimethylsilyl acetamide), methyl nitrile, and tin chloride, and then deprotected by reacting it with ammonia and methanol.

The final product is 1-ribofuranosyl 3-carboxypyridin-2-one, as depicted in Scheme 11.

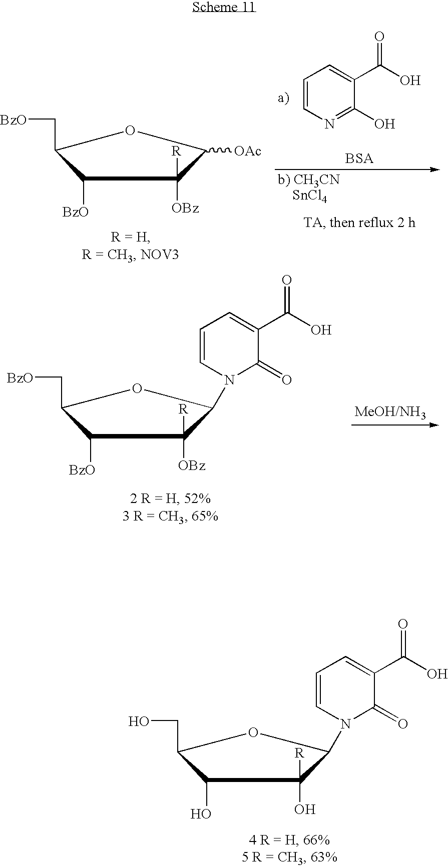

### 2) Pyridinone Carboxamide Nucleoside Analogs

A preferred synthesis for pyridinone carboxamide nucleoside analogs comprises acidic treatment of 2-hydroxynicotinic acid in the presence of methanol to give the 2-hydroxy-3-carboxylic acid methyl ester of pyridine, which is then condensed with a protected ribofuranosyl ring wherein the protective groups are as described above. For pyridinone carboxamide nucleoside analogs having a fluoro atom at C-4 of the pyridine moiety, the hydroxynicotinic acid starting material optimally has an appropriately placed fluoro atom. Alternatively, the 2-hydroxy-nicotinic acid methyl ester may be appropriately fluorinated by methods known to those skilled in the art. Deprotection with ammonia and methanol at room temperature provided 2-pyridinone carboxylic acid methyl esters, while the same treatment at elevated temperatures resulted in 2-pyridinone carboxamides, as shown in Scheme 12.

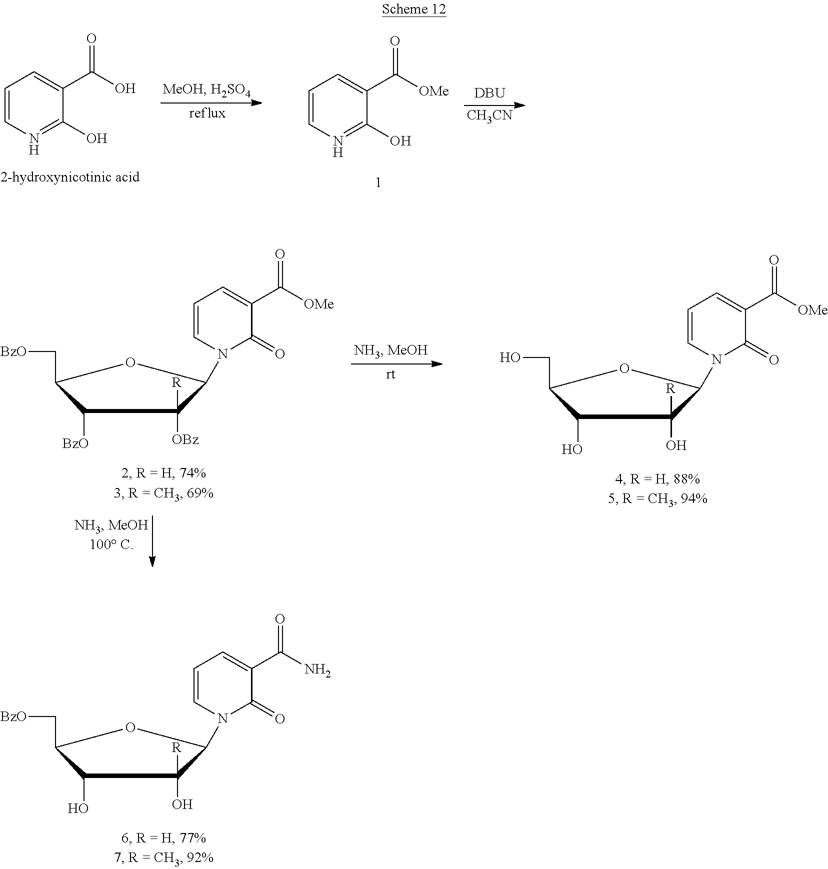

Preparation of Pyridinone Carboxamide Nucleoside Analogs is Known in the prior art, as shown in Scheme 13.

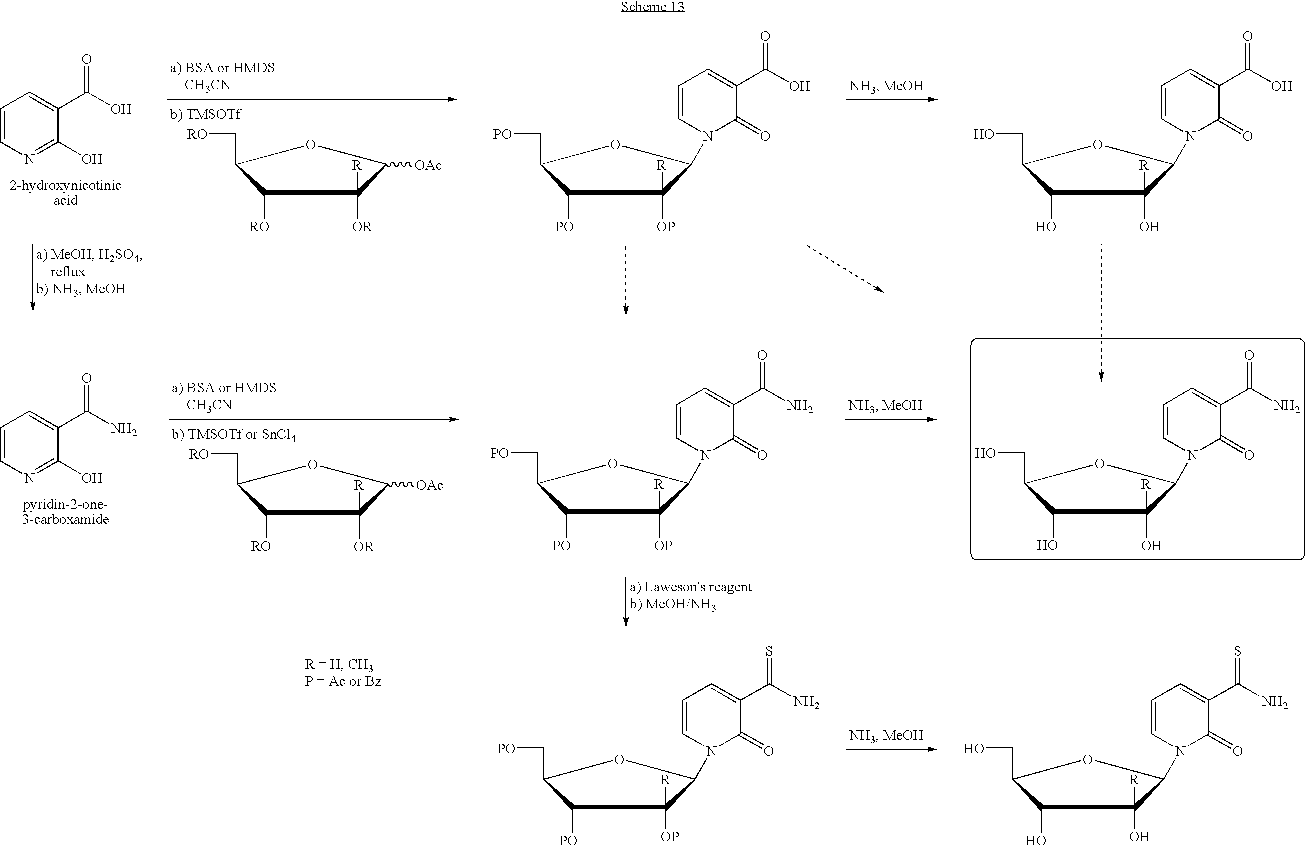

Taken from J. Heterocycl. Chem., 1989, 26(6):1931 and Nucleosides, Nucleotides & Nucleic Acids, 2001, 20(4-7):731.

### General Synthesis of Pyrimidinone Carboxamide Nucleoside Analogs

The identical synthetic steps used to prepare pyrazinone carboxamide nucleoside analogs are also used to make pyrimidinone carboxamide nucleoside analogs, except that the nucleoside base here is a pyrimidine. This is depicted in Scheme 14.

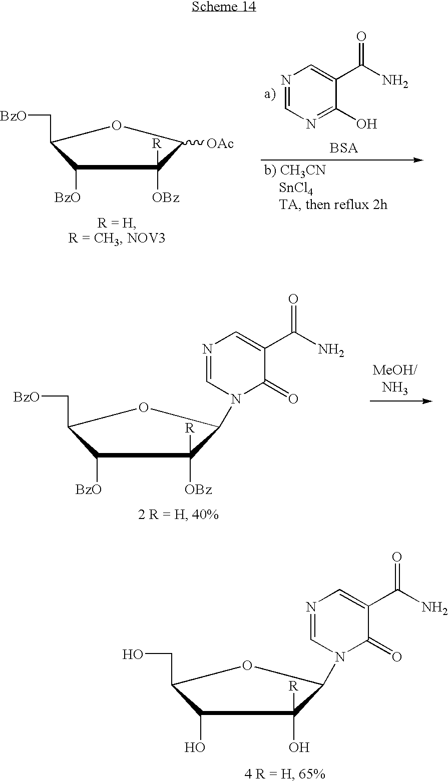

Syntheses of pyrimidinone carboxamide and pyrimidinone thioamine nucleoside analogs is known in the prior art, as shown in Scheme 15.

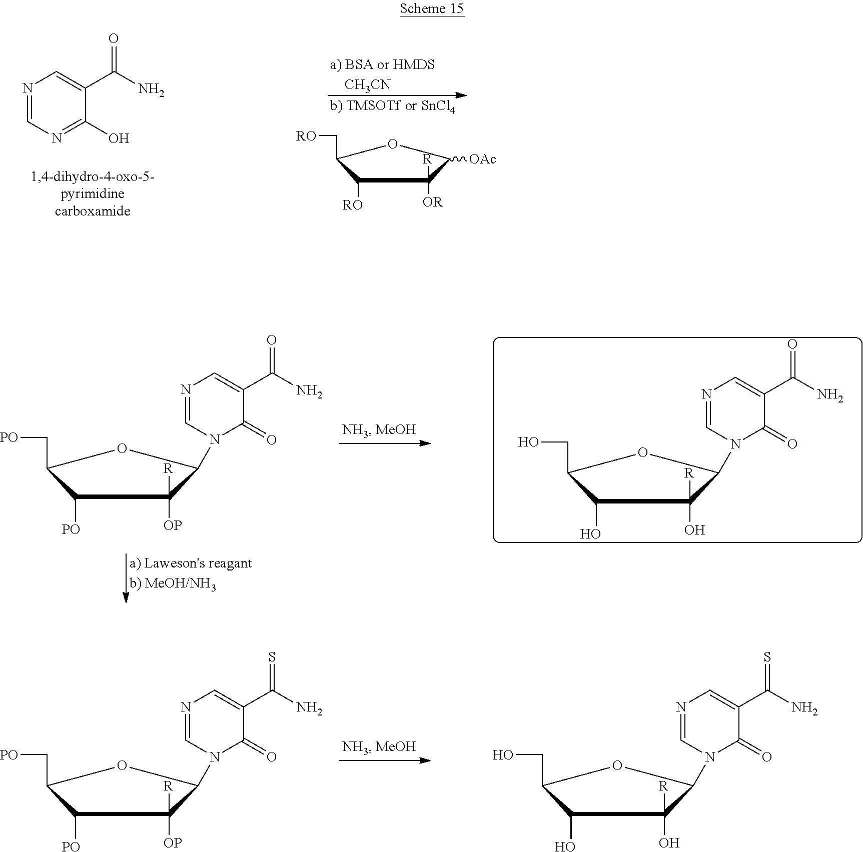

Taken from Heterocyclic Chemistry, 1989, 26(6):1931 and Nucleosides, Nucleotides & Nucleic Acids, 2001, 20(4-7):731.

General Synthesis of Triazinone Carboxamide Nucleoside Analogs

Triazinone carboxamide nucleoside analogs can be synthesized by condensing the appropriate base, such as 5-carboxylic acid-1,3,4-triazin-6-one or a 5-carboxylic acid-1,2,4 triazin-6-one, with a protected ribofuranosyl ring, wherein the protective groups are as described above in Greene et al., Protective Groups in Organic Synthesis, John Wiley and Sons, Second Edition, 1991, and known to those skilled in the art, in the presence of BSA or HMDS (hexamethyldisilazide), methyl nitrile, and tin tetrachloride or TMSOTf (trimethylsiloxy triflate) to provide the desired nucleoside analog with protective groups on the sugar ring. The protected nucleoside then can be treated with acidic methanol, followed by ammonium hydroxide to convert the carboxylic acid group on the base to a carboxamido group, and the carboxamide nucleoside analog deprotected by treatment with ammonia and methanol. This synthetic scheme is shown in Scheme 16, in which “P” denotes a protecting group.

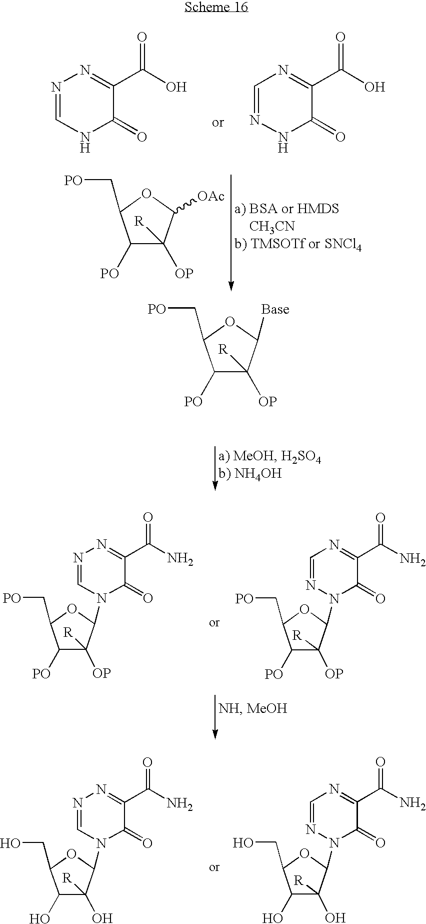

General Synthesis of Pteridine Nucleoside Analogs

N-6-ribo or 2′-C-methyl-ribofuranosyl derivative compounds that have optionally substituted pteridine nucleoside bases can be synthesized by the following process shown in Scheme 17.

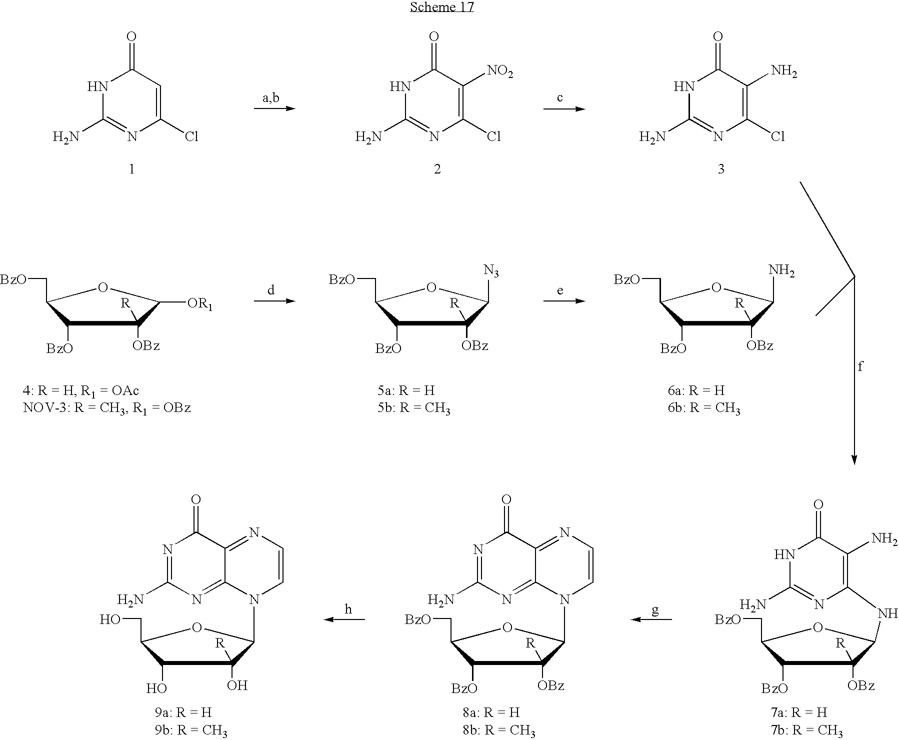

a: HNO₃/H₂SO₄ (1:1, v/), 35° C.; b: Ac₂O, cat H₂SO₄, 90° C.; c: H₂/Raney Ni, N,N-dimethylacetamide, EtOH; d: LiN₃, SDCl₄, CH₂Cl₂, r.t.; e: H₂/10% Pd/c, MeOH, AcOH; f: DBU, acetonitrile, r.t.; g: glyoxal (40% wt solution in water), sodium metabisulfite, h: MeOH/NH₃, r.t.

Synthesis of pteridine nucleoside analogs is known in the prior art. An original synthesis was taught by W. Pfleiderer et al., Chem. Bericht, 1973, 106:1952-75 and Chem. Bericht, 1961, 94:12-18, and is shown in Scheme 18.

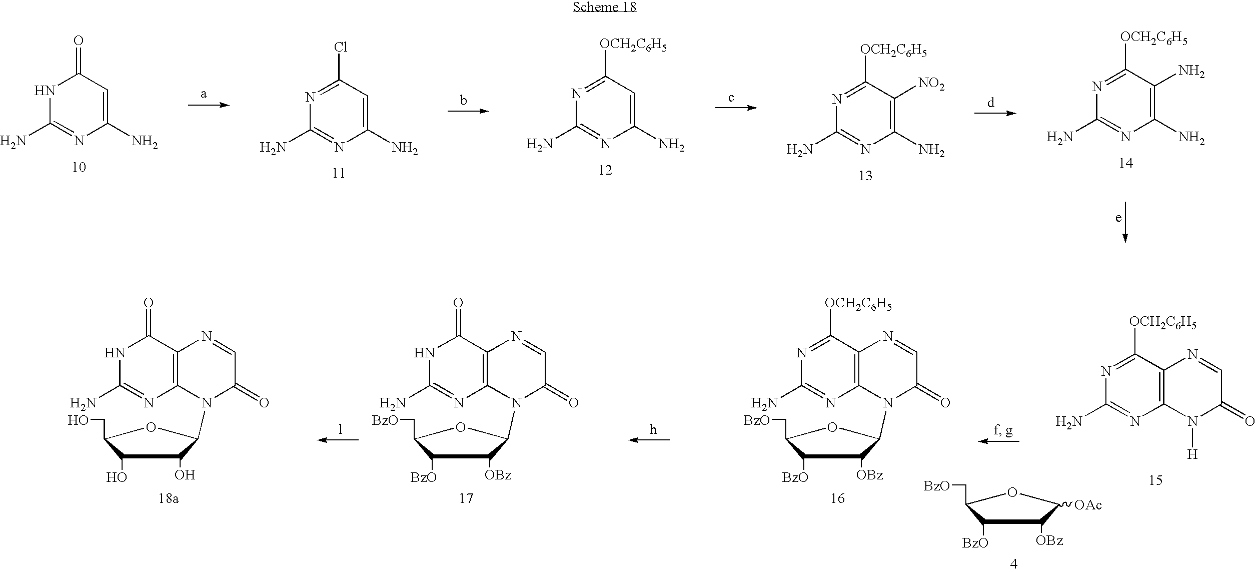

a: POCl₃, 80° C.; b: C₆H₅CH₂OH, Na, r.t.; c: HNO₃/H₂SO₄ (1:1, v/), 35°; d: H₂/Raney Ni, N,N-dimethylacetamide; e: ethyl glyoxylate diethylacetal, H₂O; f: HMDS, reflux; g: SnCl₄, CH₂Cl₂, r.t, h: H₂/10% Pd/c, MeOH, AcOH; is MeOH/NH₃, r.t.

General Synthesis of Pyridinopyrimidine Nucleoside Analogs

Ribofuranosyl derivative compounds that have optionally substituted pyridinoovrimidine nucleoside bases can be synthesized by the following process shown in Scheme 19.

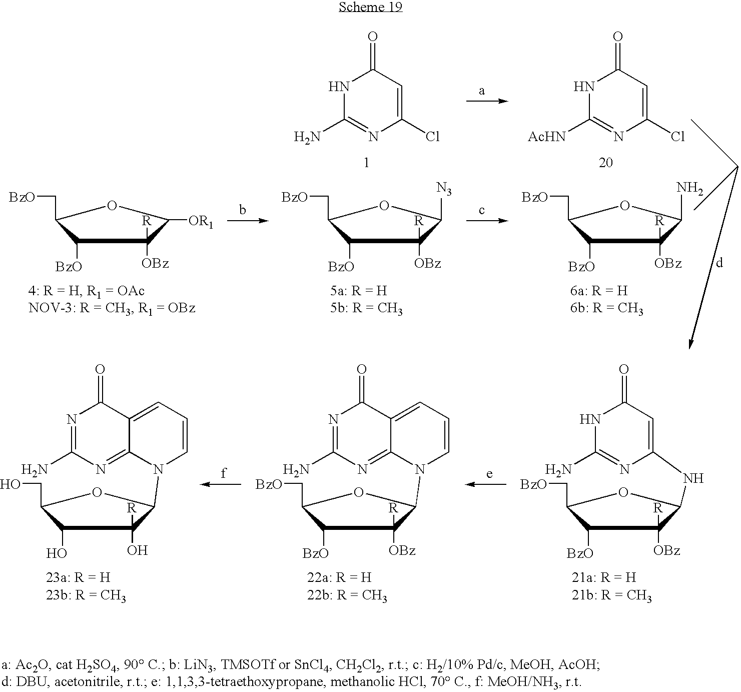

### VII. EXAMPLES

The following are non-limiting examples of the present invention.

### Example 1

### Preparation of 2-hydroxy-3-carboxamidopyrazine

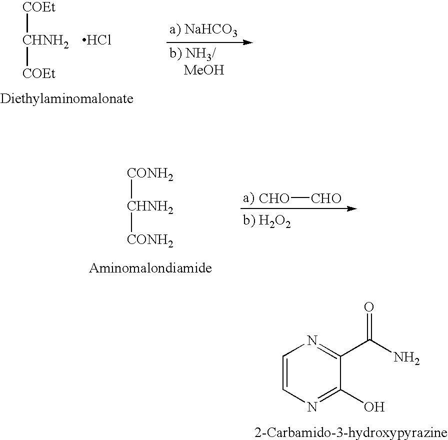

To an aqueous solution of diethylaminomalonate (hydrochloride form) was added sodium hydrogenocarbonate (pH>7). After extraction, the organic phase was evaporated under reduced pressure and treated with an ammoniacal solution of methanol at 80° C. overnight to give aminomalondiamide quantitatively. This compound was used for next step without purification and dissolved in water. To that solution was added glyoxal sodium bisulfite hemihydrate, this reaction mixture was stirred at 90° C. for 3 h, and then made basic with 58% NH₄OH. Then, 30% H₂O₂ was added dropwise with rapid stirring to the cold solution (0° C.) [J. Med. Chem. 1983, 26, 283-86, J. Heterocyclic Chem. 1979, 16, 193]. The reaction mixture was allowed to warm at room temperature and the desired 2-hydroxy-3-carboxamidopyrazine precipitated. The solid was collected (63% yield) and part of it recrystallized.

### Example 1a

### Condensation Reaction with Acylated Sugar

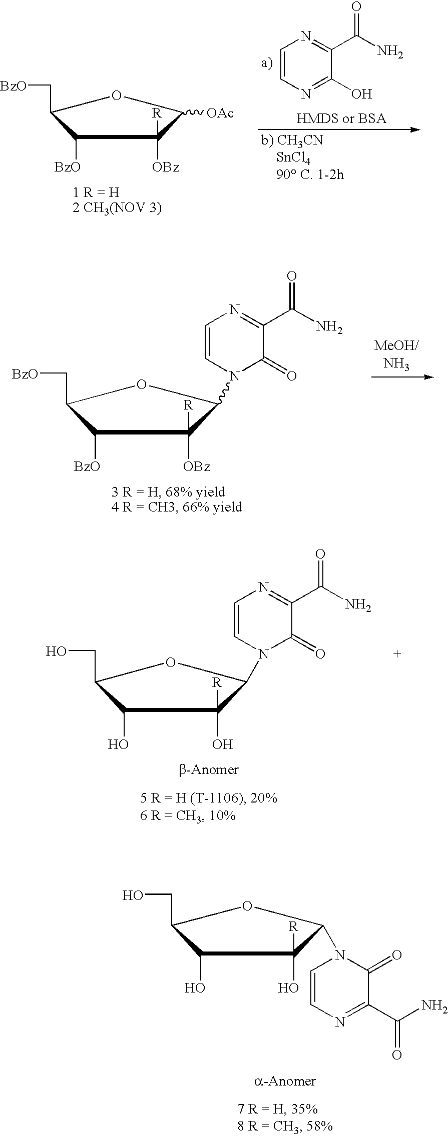

3-Hydroxy-2-pyrazinecarboxamide was silylated using hexamethyldisilazane or bis(trimethylsilyl)acetamide and treated with appropriated acylated sugars in anhydrous acetonitrile in presence of tin chloride [Toyama patent JP 2004043371 A2 20040212]. The reaction mixtures were heated at 90° C. for 1-2 h and led to anomer mixtures which couldn't be separated after silica gel column chromatography. Those anomer mixtures were debenzoylated and purified by reverse phase chromatographies to give unprotected α- and β-3-carboxamidopyrazin-2-one derivatives.

### Example 2

### Pyridinone Carboxylic Acid Nucleoside Analogs

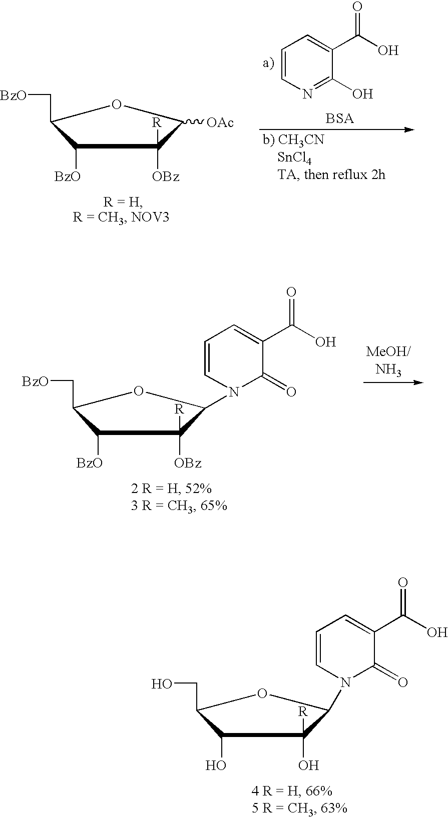

The condensation mixture was refluxed for 2 hours and 2 major compounds 2 and 3 were isolated. This reaction was described in the ribo series using either TMSOTf or tin chloride as coupling reagents [Nucleosides, Nucleotides & Nucleic acids 2001, 20 (4-7), 731; Nucleosides & Nucleotides, 1991, 10 (6), 1333]. Deprotections of 2 and 3 were quantitative and led respectively to products 4 and which were purified and recrystallized.

### Example 3

### Pyridinone Carboxamide Nucleoside Analogs

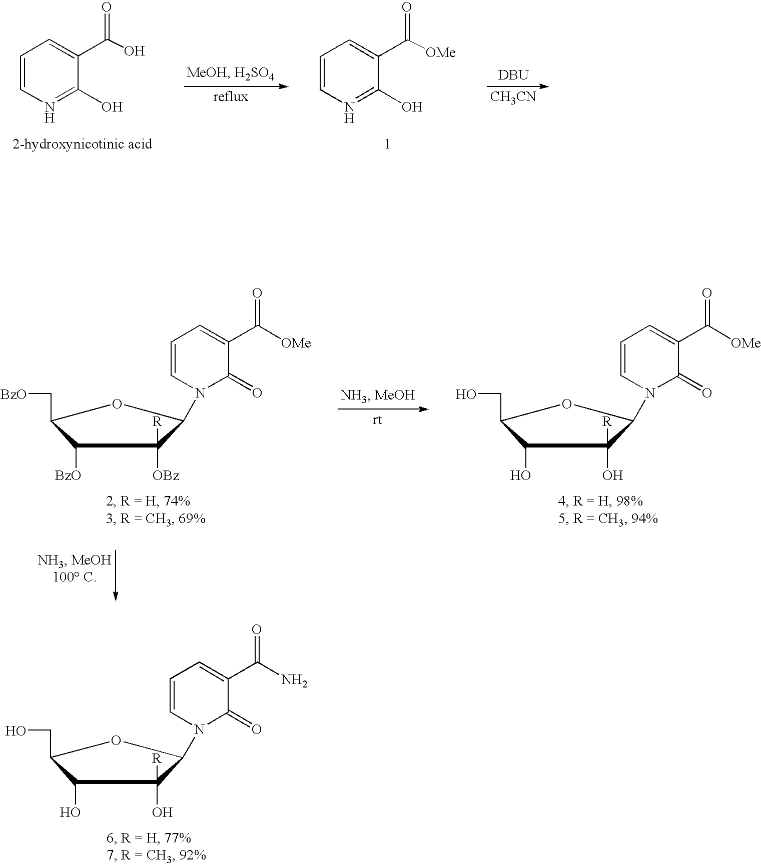

Acidic treatment [J.A.C.S. 1947, 69, 1034-37] of 2-hydroxynicotinic acid led quantitatively to the base 1 (pyridin-2-one-3-carboxylic acid methyl ester) which was condensed with acylated sugar in presence of diazabicyclo[5.4.0]undec-7-ene to give 2 and 3. Ammoniacal treatment at room temperature afforded the 2-pyrimidinone carboxylic acid methyl esters 4 and 5, while similar treatment at 1.00° C. led to the pyrimidinone carboxamide derivatives 6 and 7. All compounds have been characterized. Physical data of 6 is in accordance with data from literature [J. Heterocyclic Chem. 1989, 26, 1835] and a NOE experiment confirmed the β-anomery.

### Example 4

### Pyrimidinone Carboxamide Nucleoside Analogs

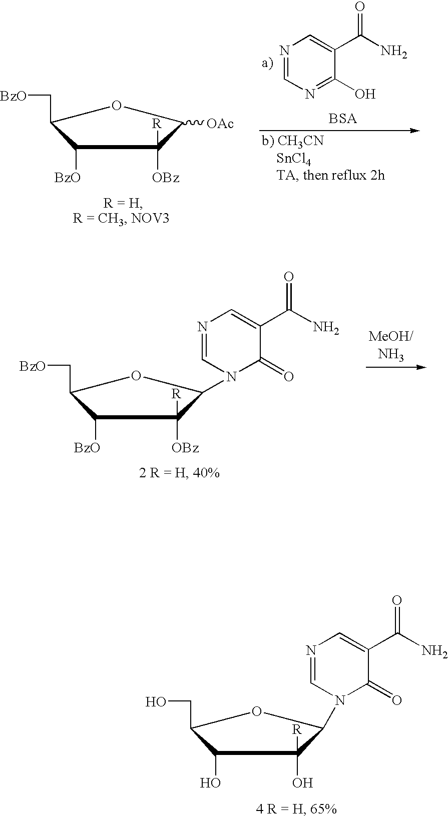

Condensation of silylated 4-hydroxy-5-pyrimidinecarboxamide with acylated sugar in presence of tin chloride in acetonitrile led to a mixture of 4 compounds. Compound 2 was isolated as the major product and deprotected to give the pyrimidinone carboxamide nucleoside analog 4.

### Example 5

### Pyridinopyrimidine Nucleoside Analogs

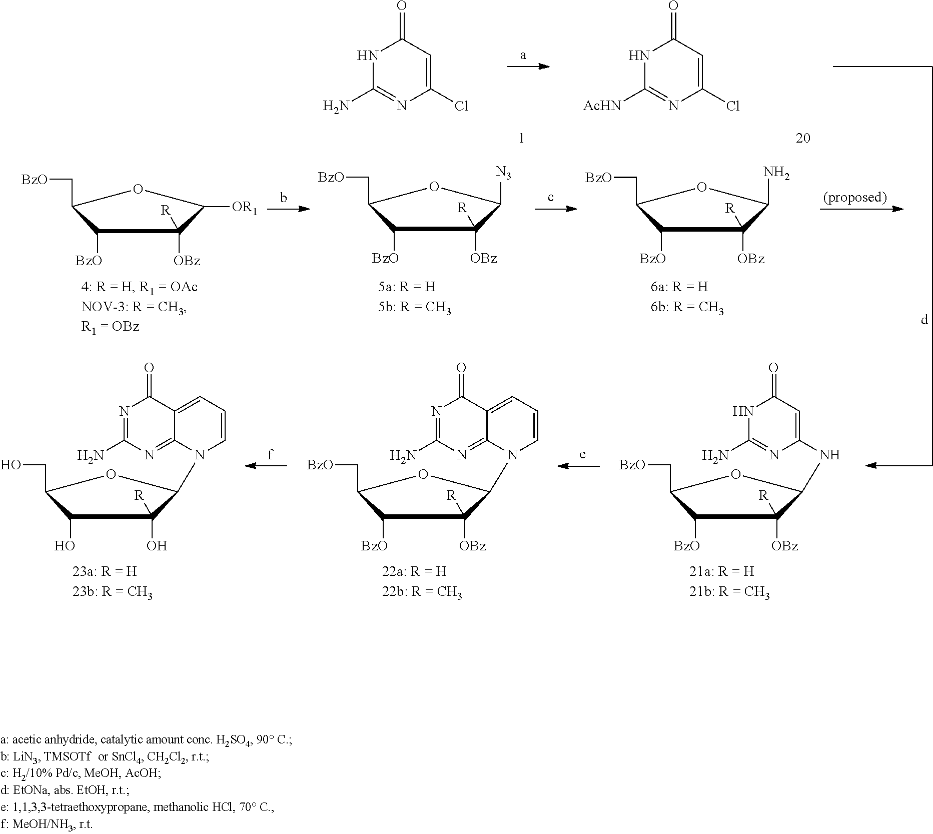

### VIII. ANTI-FLAVIVIRUS, PESTIVIRUS OR HEPACIVIRUS ACTIVITY

Compounds can exhibit anti-flavivirus, pestivirus or hepacivirus activity by inhibiting flavivirus, pestivirus or hepacivirus polymerase, by inhibiting other enzymes needed in the replication cycle, or by other pathways.

### Phosphorylation Assay of Nucleoside to Active Triphosphate

To determine the cellular metabolism of the compounds, HepG2 cells are obtained from the American Type Culture Collection (Rockville, Md.), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [³H] labeled active compound (500 dpm/pmol) for the specified time periods. The cells are maintained at 37° C. under a 5% CO₂ atmosphere. At the selected time points, the cells are washed three times with ice-cold phosphate-buffered saline (PBS). Intracellular active compound and its respective metabolites are extracted by incubating the cell pellet overnight at −20° C. with 60% methanol followed by extraction with an additional 20 μL of cold methanol for one hour in an ice bath. The extracts are then combined, dried under gentle filtered air flow and stored at −20° C. until HPLC analysis.

### Bioavailability Assay in Cynomolgus Monkeys

Within 1 week prior to the study initiation, the cynomolgus monkey is surgically implanted with a chronic venous catheter and subcutaneous venous access port (VAP) to facilitate blood collection and undergoes a physical examination including hematology and serum chemistry evaluations and the body weight is recorded. Each monkey (six total) receives approximately 250 μCi of ³H activity with each dose of active compound at a dose level of 10 mg/kg at a dose concentration of 5 mg/mL, either via an intravenous bolus (3 monkeys, IV), or via oral gavage (3 monkeys, PO). Each dosing syringe is weighed before dosing to gravimetrically determine the quantity of formulation administered. Urine samples are collected via pan catch at the designated intervals (approximately 18-0 hours pre-dose, 0-4, 4-8 and 8-12 hours post-dosage) and processed. Blood samples are collected as well (pre-dose, 0.25, 0.5, 1, 2, 3, 6, 8, 12 and 24 hours post-dosage) via the chronic venous catheter and VAP or from a peripheral vessel if the chronic venous catheter procedure should not be possible. The blood and urine samples are analyzed for the maximum concentration (Cₘₐₓ), time when the maximum concentration is achieved (Tₘₐₓ), area under the curve (AUC), half life of the dosage concentration (T_{1/2}), clearance (CL), steady state volume and distribution (Vₛₛ) and bioavailability (F).

### Bone Marrow Toxicity Assay

Human bone marrow cells are collected from normal healthy volunteers and the mononuclear population are separated by Ficoll-Hypaque gradient centrifugation as described previously by Sommadossi J-P, Carlisle R. “Toxicity of 3′-azido-3′-deoxythymidine and 9-(1,3-dihydroxy-2-propoxymethyl)guanine for normal human hematopoietic progenitor cells in vitro” Antimicrobial Agents and Chemotherapy 1987; 31:452-454; and Sommadossi J-P, Schinazi R F, Chu C K, Xie M-Y. “Comparison of cytotoxicity of the (−)- and (+)-enantiomer of 2′,3′-dideoxy-3′-thiacytidine in normal human bone marrow progenitor cells” Biochemical Pharmacology 1992; 44:1921-1925. The culture assays for CFU-GM and BFU-E are performed using a bilayer soft agar or methylcellulose method. Drugs are diluted in tissue culture medium and filtered. After 14 to 18 days at 37° C. in a humidified atmosphere of 5% CO₂ in air, colonies of greater than 50 cells are counted using an inverted microscope. The results are presented as the percent inhibition of colony formation in the presence of drug compared to solvent control cultures.

### Mitochondria Toxicity Assay

HepG2 cells are cultured in 12-well plates as described above and exposed to various concentrations of drugs as taught by Pan-Thou X-R, Cui L, Thou X-J, Sommadossi J-P, Darley-Usmer V M. “Differential effects of antiretroviral nucleoside analogs on mitochondrial function in HepG2 cells” Antimicrob Agents Chemother 2000; 44:496-503. Lactic acid levels in the culture medium after 4 day, drug exposure are measured using a Boehringer lactic acid assay kit. Lactic acid levels are normalized by cell number as measured by hemocytometer count.

### Cytotoxicity Assay

Cells are seeded at a rate of between 5×10³ and 5×10⁴/well into 96-well plates in growth medium overnight at 37° C. in a humidified CO₂ (5%) atmosphere. New growth medium containing serial dilutions of the drugs is then added. After incubation for 4 days, cultures are fixed in 50% TCA and stained with sulforhodamineB. The optical density is read at 550 nm. The cytotoxic concentration is expressed as the concentration required to reduce the cell number by 50% (CC₅₀).

### Cell Protection Assay (CPA)

The assay is performed essentially as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of test compounds are added to both infected and uninfected cells in a final concentration of 0.5% DMSO in growth medium. Each dilution is tested in quadruplicate. Cell densities and virus inocula are adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells is read in a microplate reader at 550 nm. The 50% effective concentration (EC₅₀) values are defined as the compound concentration that achieved 50% reduction of cytopathic effect of the virus.

### Plaque Reduction Assay

For each compound the effective concentration is determined in duplicate 24-well plates by plaque reduction assays. Cell monolayers are infected with 100 PFU/well of virus. Then, serial dilutions of test compounds in MEM supplemented with 2% inactivated serum and 0.75% of methyl cellulose are added to the monolayers. Cultures are further incubated at 37° C. for 3 days, then fixed with 50% ethanol and 0.8% Crystal Violet, washed and air-dried. Then plaques are counted to determine the concentration to obtain 90% virus suppression.

### Yield Reduction Assay

For each compound the concentration to obtain a 6-log reduction in viral load is determined in duplicate 24-well plates by yield reduction assays. The assay is performed as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986, with minor modifications. Briefly, MDBK cells are seeded onto 24-well plates (2×10⁵ cells per well) 24 hours before infection with BVDV (NADL strain) at a multiplicity of infection (MOI) of 0.1 PFU per cell. Serial dilutions of test compounds are added to cells in a final concentration of 0.5% DMSO in growth medium. Each dilution is tested in triplicate. After three days, cell cultures (cell monolayers and supernatants) are lysed by three freeze-thaw cycles, and virus yield is quantified by plaque assay. Briefly, MDBK cells are seeded onto 6-well plates (5×105 cells per well) 24 h before use. Cells are inoculated with 0.2 mL of test lysates for 1 hour, washed and overlaid with 0.5% agarose in growth medium. After 3 days, cell monolayers are fixed with 3.5% formaldehyde and stained with 1% crystal violet (w/v in 50% ethanol) to visualize plaques. The plaques are counted to determine the concentration to obtain a 6-log reduction in viral load.

This invention has been described with reference to its preferred embodiments. Variations and modifications of the invention, will be obvious to those skilled in the art from the foregoing detailed description of the invention.

What is claimed is:

1. A method for treating a pestivirus, flavivirus or hepacivirus infection in a host comprising administering to said host an effective amount of a nucleoside compound of Formula (i) 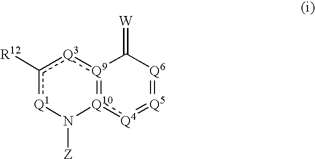
or a pharmaceutically acceptable salt or prodrug thereof, wherein:
W is O;
Q¹ is C—R where R is H or halogen;
Q³ is C—R where R is H or halogen, preferably F;
Q⁴ and Q⁶ each independently is N, C—H, or N—H;
Q⁵ is C—R where R is NR⁴R⁵, NHR⁴, or NH₂
Q⁹ and Q¹⁰ each independently is C;
Z is Formula (IV), 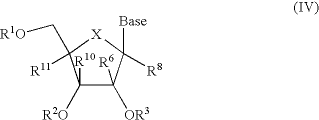
wherein X is O, S or N—H;
R¹, R², and R³ each independently is H, optionally substituted phosphate or phosphonate, acyl, alkyl, or amino acid;
R⁸ and R¹¹ each independently is H, hydroxyl, alkyl, alkenyl, alkynyl, chloro, bromo, fluoro, iodo, or O(alkyl); and
R⁶ and R¹⁰ each independently is H, alkyl or halo substituted alkyl, Cl, F, Br, or I;
R¹² is H; and
Each R⁴ and R⁵ independently is H, acyl, or alkyl.

2. The method of claim 1 wherein:
Q¹ is C—R where R is H;
Q³ is C—R where R is halogen;
Q⁴ and Q⁶ each independently is N;
Z is Formula (IV), wherein X is O; R¹, R², R³, R⁸, R¹⁰ and R¹¹ each independently is H; and R⁶ is lower alkyl.

3. A method for the treatment of a host infected with a flavivirus, pestivirus or hepacivirus infection comprising administering to said host an effective treatment amount of a compound of Formula (xviii) 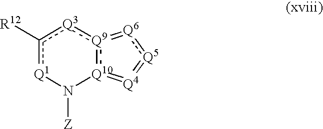
or a pharmaceutically acceptable salt or prodrug thereof, wherein:
Q¹, Q⁴ and Q⁶ each independently is C—R or N;
Q³ and Q⁵ each independently is C—R or N;
Q⁹ and Q¹⁰ each independently is C;
Z is Formula (III), 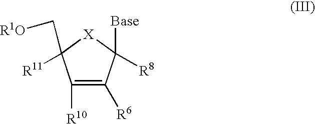
wherein X is O, S or N—R where R is H;
R¹ is H, optionally substituted phosphate or phosphonate, acyl, alkyl, or amino acid;
R⁶ and R¹⁰ is H, alkyl or halo substituted alkyl, chloro, bromo, fluoro, or iodo;
R⁸ and R¹¹ each independently is H, OH, alkyl, alkenyl, alkynyl, chloro, bromo, fluoro, iodo, or O(alkyl);
R¹² is H; and
R is each independently H, halo, alkyl, alkenyl, alkynyl, alkoxy, alkoxyalkyl, hydroxyalkyl, cycloalkyl, nitro, cyano, OH, ether, NH₂, amide, SH, thioalkyl, CF₃, CH₂OH, CH₂F, CH₂Cl, CH₂CF₃, C(═O)OH, C(═O)Oalkyl or aryl, C(═O)-alkyl, C(═O)-aryl, C(═O)-alkoxyalkyl, C(═O)NH₂, C(═O)NHalkyl, C(═O)N(alkyl)₂, or N₃.

4. The method of claim 3, wherein:
Q¹, Q⁴ and Q⁶ each independently is C—R;
Q³ and Q⁵ each independently is N;
X is O;
R¹ is H;
R⁸ and R¹¹ each independently is H or lower alkyl;
R⁶ is lower alkyl; and
R¹⁰ is H or alkyl.

5. A method for the treatment of a host infected with a flavivirus, pestivirus or hepacivirus infection comprising administering to the host an effective treatment amount of a compound of Formula (B) 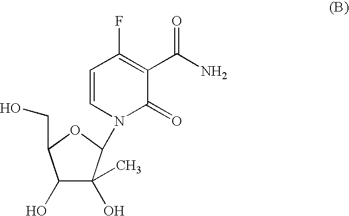
or a pharmaceutically acceptable salt or ester thereof, optionally in a pharmaceutically acceptable carrier.

6. A method for the treatment of a host infected with a flavivirus, pestivirus or hepacivirus infection comprising administering to the host an effective treatment amount of a compound of Formula (C) 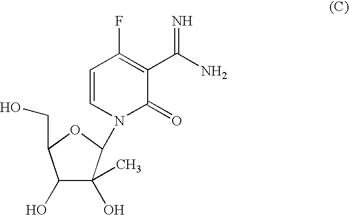
or a pharmaceutically acceptable salt or ester thereof, optionally in a pharmaceutically acceptable carrier.

7. A method for the treatment of a host infected with a flavivirus, pestivirus or hepacivirus infection comprising administering to the host an effective treatment amount of a compound of Formula (E) 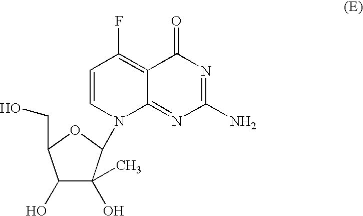
or a pharmaceutically acceptable salt or ester thereof, optionally in a pharmaceutically acceptable carrier.

8. A method for the treatment of a host infected with a flavivirus, pestivirus or hepacivirus infection comprising administering to the host an effective treatment amount of a compound of Formula (M) 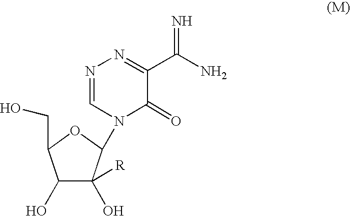
or a pharmaceutically acceptable salt or ester thereof, optionally in a pharmaceutically acceptable carrier.

9. A method for the treatment of a host infected with a flavivirus, pestivirus or hepacivirus infection comprising administering to the host an effective treatment amount of a compound of Formula (N) 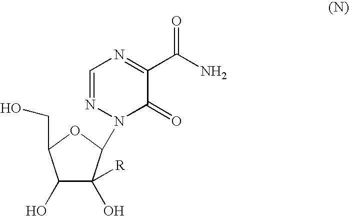
or a pharmaceutically acceptable salt or ester thereof, optionally in a pharmaceutically acceptable carrier.

10. A method for the treatment of a host infected with a flavivirus, pestivirus or hepacivirus infection comprising administering to the host an effective treatment amount of a compound of Formula (O) 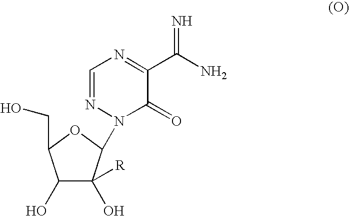
or a pharmaceutically acceptable salt or ester thereof, optionally in a pharmaceutically acceptable carrier.

11. A method for the treatment of a host infected with a flavivirus, pestivirus or hepacivirus infection comprising administering to the host an effective treatment amount of a compound of Formula (Q) 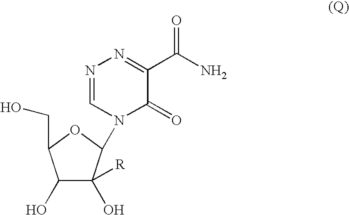
or a pharmaceutically acceptable salt or ester thereof, optionally in a pharmaceutically acceptable carrier.

12. A compound of Formula (B) 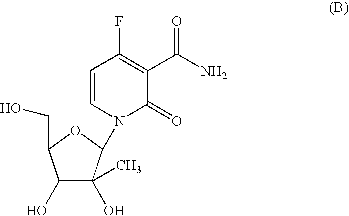
or a pharmaceutically acceptable salt or ester thereof.

13. A compound of Formula (C) 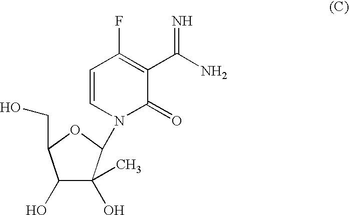
or a pharmaceutically acceptable salt or ester thereof.

14. A compound of Formula (E) 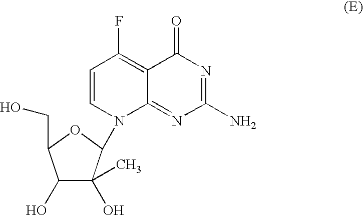
or a pharmaceutically acceptable salt or ester thereof.

15. A compound of Formula (M) 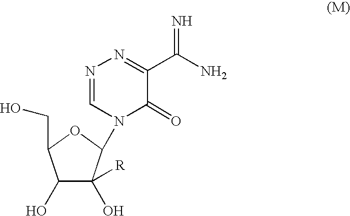
or a pharmaceutically acceptable salt or ester thereof.

16. A compound of Formula (N) 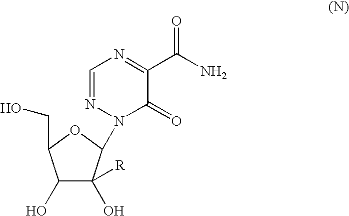
or a pharmaceutically acceptable salt or ester thereof.

17. A compound of Formula (O) 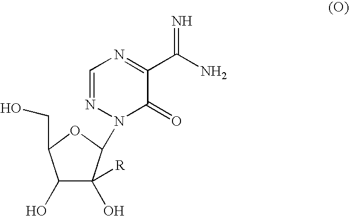
or a pharmaceutically acceptable salt or ester thereof.

18. A compound of Formula (Q) 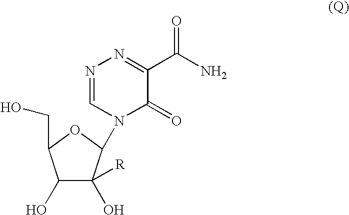
or a pharmaceutically acceptable salt or ester thereof.

19. A pharmaceutical composition comprising a compound of any one of claims 12-18, or a pharmaceutically acceptable salt or ester thereof, and a pharmaceutically acceptable carrier.

20. Use of a compound of any one of claims 12-18 or a pharmaceutically acceptable salt or ester thereof, optionally in a pharmaceutically acceptable carrier, in a method for the treatment of a host infected with a flavivirus, pestivirus or hepacivirus infection.

21. Use of a compound of any one of claims 12-18 or a pharmaceutically acceptable salt or ester thereof, optionally in a pharmaceutically acceptable carrier, in the manufacture of a medicament for the treatment of a host infected with a flavivirus, pestivirus or hepacivirus infection.
